(19) United States
(12) Patent Application Publication
(10) Pub. No.: US 2022/0241394 A1
(43) Pub. Date: Aug. 4, 2022

(54) TREATMENT OF DISEASES ASSOCIATED WITH PROTEIN MISFOLDING BY NERVOUS SYSTEM EXPRESSION OF AN ENZYME WHICH HAS A DEOXYRIBONUCLEASE (DNase) ACTIVITY

(71) Applicant: CLS THERAPEUTICS LIMITED, St. Peter Port (GG)

(72) Inventors: Dmitry Dmitrievich GENKIN, Saint Petersburg (RU); Georgy Viktorovich TETS, New York, NY (US); Viktor Veniaminovich TETS, New York, NY (US)

(73) Assignee: CLS THERAPEUTICS LIMITED, St. Peter Port (GG)

(21) Appl. No.: 17/626,616

(22) PCT Filed: Jul. 10, 2020
(86) PCT No.: PCT/US20/41579

§ 371 Date: Jan. 12, 2022

Related U.S. Application Data
(60) Provisional application No. 62/873,840, filed on Jul. 12, 2019.

(51) Int. Cl. A61K 39/12; A61K 48/00; A61P 25/28; C12N 15/86; C12N 9/22; C12N 7/00

(52) U.S. Cl. CPC A61K 39/12; A61K 48/0058; A61K 48/0075; C12N 7/00; C12N 15/86; C12N 9/22; A61P 25/28

(57) ABSTRACT

The invention relates to the nervous system-specific delivery and/or expression of an enzyme which has a deoxyribonuclease (DNase) activity for enhanced clearance of microbial and viral cell free DNA (cfDNA) accumulated in cerebrospinal fluid (CSF), brain and other parts of nervous system, with the use of such nervous-system specific delivery and/or expression for treatment of various diseases associated with protein misfolding.

### CROSS-REFERENCE TO RELATED APPLICATIONS

This application claims priority to U.S. Provisional Patent Application No. 62/873,840, filed Jul. 12, 2019, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 10, 2020, is named 252732_000009_SL.txt and is 92,498 bytes in size.

### FIELD OF THE INVENTION

The invention relates to the nervous system-specific delivery and/or expression of an enzyme which has a deoxyribonuclease (DNase) activity for enhanced clearance of microbial and viral cell free DNA (cfDNA) accumulated in cerebrospinal fluid (CSF), brain and other parts of nervous system, with the use of such nervous-system specific delivery and/or expression for treatment of various diseases associated with protein misfolding.

### BACKGROUND OF THE INVENTION

The pathogenesis of distinct neurodegenerative diseases, autoimmune diseases and tumors is characterized by misfolding of the proteins with prion-like properties such as β-amyloid and Tau in Alzheimer's disease (AD) and taupathias, alpha-synuclein in Parkinson's disease, TDP-43 and SOD1 in amyotrophic lateral sclerosis, IAPP in type 1 diabetes and others. Despite the fact that these proteins associated with different diseases are distinct the formation of their misfolded forms into prion proteins (PrPs) aggregates possessing neurotoxicity enables sharing of the similar progressive way of these pathologies. PrPs are characterized by self-propagation, undergoing a conformational switch from one conformational state to another, which leads to the creation of new prions and are transmissible from cell to cell, enabling disease progression (Prusiner et al., Biochemistry. 1982, 21(26):6942-50).

The present inventors have previously demonstrated that systemic administration of high doses of deoxyribonuclease (DNase) protein into a patient's circulation can be useful for treatment of a number of diseases and conditions associated with increased levels of cfDNA in the blood, including cancers (e.g., carcinomas, sarcomas, lymphomas, melanoma; see, e.g., U.S. Pat. Nos. 7,612,032; 8,710,012; 9,248,166), development of somatic mosaicism (see, e.g., U.S. Pat. Appl. Pub. No. US20170056482), side effects associated with a chemotherapy or a radiation therapy (see, e.g., U.S. Pat. Appl. Pub. No. US20170100463), neurodegenerative diseases (see, e.g., Int. Appl. Pub. No. WO2016/190780), infections (see, e.g., U.S. Pat. Nos. 8,431,123 and 9,072,733), diabetes (see, e.g., U.S. Pat. No. 8,388,951), atherosclerosis (see, e.g., U.S. Pat. No. 8,388,951), stroke (see, e.g., U.S. Pat. No. 8,796,004), angina (see, e.g., U.S. Pat. No. 8,796,004), ischemia (see, e.g., U.S. Pat. No. 8,796,004), kidney damage (see, e.g., U.S. Pat. No. 9,770,492), delayed-type hypersensitivity reactions such as, e.g., graft-versus-host disease [GVHD]) (see, e.g., U.S. Pat. No. 8,535,663), reduction of fertility (see, e.g., U.S. Pat. No. 8,916,151), age-specific sperm motility impairment (see, e.g., U.S. Pat. No. 8,871,200), and aging (see, e.g., U.S. Pat. Appl. Pub. No. US20150110769). All of these patents and applications are incorporated by reference herein in their entireties.

While systemic administration of DNase protein appears to be useful for treating diseases and conditions associated with increased amount of circulating cfDNA, in clinical settings when protein misfolding is caused by microbial or viral DNA systemic treatment with DNase protein showed limited effects (see, e.g., Int. Appl. Pub. No. W2014/020564).

### SUMMARY OF THE INVENTION

The present invention is based on unexpected discovery that cell free DNA (cfDNA) of microbial (e.g., bacterial, fungal, intracellular or extracellular parasites) and viral (e.g., bacteriophages, eukaryotic viruses) origin trigger aggregation and misfolding of proteins, including proteins with prion-like properties both intercellulary and extracellularly within nervous tissues much faster compared to human DNA. As demonstrated herein, the intracerebrospinal injection of bacterial DNA to 3×TG mice, MitoPark mouse, SOD1-G93A leads to significantly aggressive and fast development of Alzheimer's disease, Parkinson's disease and ALS compared to those triggered by the injection of human DNA. Importantly, certain neurodegenerative pathologies that are characterized with protein misfolding following microbial and viral DNA exposure, do not have elevated cfDNA levels.

As further disclosed herein, microbial (e.g., bacterial, fungal, intracellular or extracellular parasites) and viral (e.g., bacteriophages, eukaryotic viruses) cfDNA released within intestine trigger formation of complexes between cfDNA and prion-like proteins of microbial and viral (e.g. bacteriophage) origin. Such complexes can be found in intestine between apical surface of intestinal epithelial cells and intestinal mucus gel layer as well as in blood and nervous system (e.g., in mice suffering from neurodegeneration as part of larger complexes of misfolded proteins of neural origin).

As specified in the Background section, above, there is a need for more efficient methods of reduction of circulating cfDNA levels so as to increase the effectiveness of treating diseases and conditions associated with protein misfolding in the cerebrospinal fluid (CSF) or other components of the nervous system (including both central nervous system [CNS] and peripheral nervous system, including enteric nervous system [ENS]) that are caused by protein misfolding due to the local presence of microbial (e.g., bacterial, fungal, intracellular or extracellular parasites) and/or viral (e.g. bacteriophages, eukaryotic viruses) cfDNA such as, e.g., neurodegenerative diseases, neurodevelopmental diseases, psychiatric diseases, autoimmune diseases, nervous system tumors, and infections. The present invention addresses these and other needs by providing methods and compositions for nervous system-specific delivery and/or expression of enzymes which have a deoxyribonuclease (DNase) activity activity. According to the present invention, nervous system-specific delivery and/or expression of enzymes which have a DNase activity can be achieved using various delivery vehicles, including, without limitation, viral vectors (e.g., adeno-associated virus vectors, adenovirus vectors, retrovirus vectors [e.g., lentivirus vectors]), nanoparticles (e.g., phosphoramidite nanoparticles), liposomes (e.g., cationic liposomes such as, e.g., N-[1-(2,3-dioleyloxy)propyl]-N,N,N-trimethylammonium chloride (DOTMA)), lactoferrin, poly L-lysine, polyethyleneimine, chitosan, etc.), naked DNA, etc.

In one aspect, the invention provides a recombinant adeno-associated virus (rAAV) expression vector comprising (i) a capsid protein and (ii) a nucleic acid comprising a promoter operably linked to a nucleotide sequence encoding an enzyme which has a deoxyribonuclease (DNase) activity, wherein the promoter is a nervous system-specific promoter. In one embodiment, the AAV is selected from serotype 1 (AAV1), AAV2, AAV3, AAV4, AAV5, AAV6, AAV7, AAV5, AAV9, AAV10, AAV11, AAV12, AAVrh10, AAVLK03, AAVLK06, AAVLK12, AAV-KP1, AAV-F, AAVDJ, AAV-PHP.B, AAVhu37, AAVrh64R1, and Anc 80. In one embodiment, the AAV is from serotype 5 (AAV5). In one embodiment, the AAV is from serotype 9 (AAV9). In one embodiment, the AAV is from serotype Anc 80. In one embodiment, the capsid protein comprises one or more mutations which improve efficiency and/or specificity of the delivery of the vector to the nervous system as compared to the corresponding wild-type capsid protein. In one embodiment, the improved efficiency and/or specificity of the delivery of the vector to the nervous system results in a substantially increased expression of the enzyme in the nervous system as compared to other tissues and organs. In one embodiment, the one or more mutations in the capsid protein are selected from the group consisting of S279A, S671A, K137R, T252A, and any combinations thereof In one embodiment, the capsid protein comprises the sequence SEQ ID NO:34. In one embodiment, the capsid protein consists of the sequence SEQ ID NO:34. In one embodiment, the capsid protein comprises the sequence SEQ ID NO: 35. In one embodiment, the capsid protein consists of the sequence SEQ ID NO: 35. In one embodiment, the nucleic acid further comprises two AAV inverted terminal repeats (ITRs), wherein the ITRs flank the nucleotide sequence encoding the enzyme which has a DNase activity.

In another aspect, the invention provides a recombinant retroviral vector comprising (i) a capsid protein and (ii) a nucleic acid comprising a promoter operably linked to a nucleotide sequence encoding an enzyme which has a deoxyribonuclease (DNase) activity, wherein the promoter is a nervous system-specific promoter. In one embodiment, the vector is a lentiviral vector.

In one embodiment of any of the above vectors, the nervous system-specific promoter mediates a substantially increased expression of the enzyme in the nervous system as compared to other tissues and organs.

In one embodiment of any of the above vectors, the nervous system-specific promoter is a microglia-specific promoter.

In one embodiment of any of the above vectors, the nervous system-specific promoter is a neuron-specific promoter.

In one embodiment of any of the above vectors, the nervous system-specific promoter is selected from the group consisting of F4/80 promoter, CD68 promoter, TMEM119 promoter, CX3CR1 promoter, CMV promoter, MEF2 promoter, FoxP2 promoter, Iba1 promoter, TTR promoter, CD1lb promoter, c-fes promoter, NSE promoter, synapsin promoter, CamKII promoter, α-CaM KII promoter, VGLUT1 promoter, and glial fibrillary acidic protein (GFAP) promoter. In one embodiment, the nervous system-specific promoter is a F4/80 promoter. In one embodiment, the F4/80 promoter comprises the sequence SEQ ID NO: 38. In one embodiment, the F4/80 promoter consists of the sequence SEQ ID NO: 38. In one embodiment, the nervous system-specific promoter is a CMV promoter. In one embodiment, the CMV promoter comprises the sequence SEQ ID NO: 37. In one embodiment, the CMV promoter consists of the sequence SEQ ID NO: 37. In one embodiment, the nervous system-specific promoter is a TMEM119 promoter. In one embodiment, the TMEM119 promoter comprises the sequence SEQ ID NO: 39. In one embodiment, the TMEM119 promoter consists of the sequence SEQ ID NO: 39. In one embodiment, the nervous system-specific promoter is a MEF2 promoter. In one embodiment, the MEF2 promoter comprises the sequence SEQ ID NO: 40. In one embodiment, the MEF2 promoter consists of the sequence SEQ ID NO: 40. In one embodiment, the nervous system-specific promoter is a FoxP2 promoter. In one embodiment, the FoxP2 promoter comprises the sequence SEQ ID NO: 41. In one embodiment, the FoxP2 promoter consists of the sequence SEQ ID NO: 41. In one embodiment, the nervous system-specific promoter is a synapsin promoter. In one embodiment, the synapsin promoter comprises the sequence SEQ ID NO: 36. In one embodiment,

the synapsin promoter consists of the sequence SEQ ID NO: 36.

In one embodiment of any of the above vectors, the enzyme which has a DNase activity is selected from the group consisting of DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), phosphodiesterase I, lactoferrin, acetylcholinesterase, and mutants or derivatives thereof. In one embodiment, the enzyme which has a DNase activity is DNase I or a mutant or derivative thereof In one embodiment, the DNase I is a human DNase I or a mutant or derivative thereof In one embodiment, the DNase I mutant comprises one or more mutations in an actin binding site. In one embodiment, the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof. In one embodiment, one of the mutations in the actin-binding site is a mutation at Ala-114. In one embodiment, the DNase I mutant comprises one or more mutations increasing DNase activity. In one embodiment, one or more mutations increasing DNase activity are selected from the group consisting of Q9R, E13R, E13K, T14R, T14K, H44R, H44K, N74K, A114F, and any combinations thereof. In one embodiment, one or more mutations increasing DNase activity are selected from the group consisting of Q9R, E13R, N74K and A114F, and any combinations thereof. In one embodiment, the DNase I mutant comprises the mutations Q9R, E13R, N74K, and A114F. In one embodiment, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P70T, S94N:Y96S, S94N:Y96T, and any combinations thereof. In one embodiment, the DNase I mutant comprises a sequence having at least 80% sequence identity to the sequence of SEQ ID NO: 5. In one embodiment, the DNase I mutant comprises the sequence SEQ ID NO: 5. In one embodiment, the DNase I mutant consists of the sequence SEQ ID NO: 5. In one embodiment, the nucleic acid comprises a nucleotide sequence which is at least 85% or at least 90% or at least 95% identical to SEQ ID NO: 21. In one embodiment, the nucleic acid comprises the nucleotide sequence SEQ ID NO: 21. In one embodiment, the DNase I mutant comprises a sequence having at least 80% or at least 85% or at least 90% or at least 95% sequence identity to the sequence of SEQ ID NO: 2. In one embodiment, the DNase I mutant comprises the sequence SEQ ID NO: 2. In one embodiment, the DNase I mutant consists of the sequence SEQ ID NO: 2. In one embodiment, the nucleic acid comprises a nucleotide sequence which is at least 85% or at least 90% or at least 95% identical to SEQ ID NO: 19. In one embodiment, the nucleic acid comprises the nucleotide sequence SEQ ID NO: 19. In one embodiment, the DNase I comprises the sequence SEQ ID NO: 4. In one embodiment, the DNase I consists of the sequence SEQ ID NO: 4. In one embodiment, the nucleic acid comprises a nucleotide sequence which is at least 85% or at least 90% or at least 95% identical to SEQ ID NO: 23. In one embodiment, the nucleic acid comprises the nucleotide sequence SEQ ID NO: 23. In one embodiment, the DNase I comprises the sequence SEQ ID NO: 1. In one embodiment, the DNase I consists of the sequence SEQ ID NO: 1. In one embodiment, the nucleic acid comprises a nucleotide sequence which is at least 85% or at least 90% or at least 95% identical to SEQ ID NO: 22. In one embodiment, the nucleic acid comprises the nucleotide sequence SEQ ID NO: 22. In one embodiment, the nucleic acid comprises a nucleotide sequence which is at least 85% identical to SEQ ID NO: 32. In one embodiment, the nucleic acid comprises the nucleotide sequence SEQ ID NO: 32.

In one embodiment of any of the vectors of the invention, the enzyme which has a DNase activity is a fusion protein comprising (i) a DNase enzyme or a fragment thereof linked to (ii) an albumin or an Fc or a fragment thereof.

In one embodiment of any of the vectors of the invention, the sequence encoding the enzyme which has a DNase activity comprises a sequence encoding a secretory signal sequence, wherein said secretory signal sequence mediates effective secretion of the enzyme. In one embodiment, the secretory signal sequence is selected from the group consisting of DNase I secretory signal sequence, IL2 secretory signal sequence, the albumin secretory signal sequence, the β-glucuronidase secretory signal sequence, the alkaline protease secretory signal sequence, and the fibronectin secretory signal sequence. In one embodiment the secretory signal sequence comprises the sequence MRGMKLLGALLALAALLQGAVS (SEQ ID NO: 6) or MYRMQLLSCIALSLALVTNS (SEQ ID NO: 7). In one embodiment the secretory signal sequence consists of the sequence MRGMKLLGALLALAALLQGAVS (SEQ ID NO: 6) or MYRMQLLSCIALSLALVTNS (SEQ ID NO: 7). In one embodiment, the secretory signal sequence comprises a sequence having at least 80% or at least 85% or at least 90% or at least 95% sequence identity to the sequence of MRGMKLLGALLALAALLQGAVS (SEQ ID NO: 6) or a sequence having at least 85% or at least 90% or at least 95% sequence identity to the sequence of MYRMQLLSCIALSLALVTNS (SEQ ID NO: 7). In one embodiment, the sequence encoding the secretory signal sequence comprises a nucleotide sequence which is at least 85% or at least 90% or at least 95% identical to SEQ ID NO: 20. In one embodiment, the sequence encoding the secretory signal sequence comprises the nucleotide sequence SEQ ID NO: 20. In one embodiment, the secretory signal sequence comprises the sequence MRYTGLMGTLLTLVNLLQLAGT (SEQ ID NO: 25). In one embodiment, the secretory signal sequence consists of the sequence MRYTGLMGTLLTLVNLLQLAGT (SEQ ID NO: 25). In one embodiment, the secretory signal sequence comprises a sequence having at least 80% or at least 85% or at least 90% or at least 95% sequence identity to the sequence of MRYTGLMGTLLTLVNLLQLAGT (SEQ ID NO: 25). In one embodiment, the sequence encoding the secretory signal sequence comprises a nucleotide sequence which is at least 85% or at least 90% or at least 95% identical to SEQ ID NO: 27. In one embodiment, the sequence encoding the secretory signal sequence comprises the nucleotide sequence SEQ ID NO: 27.

In one embodiment of any of the above vectors, the nucleic acid further comprises one or more enhancers located upstream or downstream of the promoter. In one embodiment, the one or more enhancers are selected from the group consisting of nPE2 enhancer, Gal4 enhancer, foxP2 enhancer, Mef2 enhancer, and any combination thereof.

In one embodiment of any of the above vectors, the nucleic acid further comprises a polyadenylation signal operably linked to the nucleotide sequence encoding the enzyme which has a DNase activity.

In one embodiment of any of the above vectors, the nucleic acid further comprises a Kozak sequence. In one embodiment, the Kozak sequence comprises the sequence 5′-GCCGCCACC-3′ (SEQ ID NO: 33).

In one embodiment of any of the above vectors, the nucleic acid further comprises a post-transcriptional regulatory element. In one embodiment, the post-transcriptional regulatory element is a woodchuck hepatitis post-transcriptional regulatory element (WPRE).

In a separate embodiment, the invention provides pharmaceutical compositions comprising any of the above vectors and a pharmaceutically acceptable carrier and/or excipient.

In another aspect, the invention provides a method for delivering an enzyme which has a deoxyribonuclease (DNase) activity to the nervous system in a subject in need thereof, comprising administering to the subject any of the above vectors or compositions.

In another aspect, the invention provides a method for treating a disease associated with protein misfolding in a subject in need thereof, said method comprising administering to the subject a therapeutically effective amount of any of the above vectors or compositions. In one embodiment, the protein misfolding is associated with the presence of cell-free DNA (cfDNA) in cerebrospinal fluid (CSF) or nervous system tissue(s). In one embodiment, the cfDNA is microbial and/or viral cfDNA. In one embodiment, the microbial cfDNA is bacterial cfDNA. In one embodiment, the viral cfDNA is bacteriophage cfDNA. In one embodiment, the disease associated with protein misfolding is selected from neurodegenerative diseases, neurodevelopmental diseases, psychiatric diseases, autoimmune diseases, oncological diseases and infections. In one embodiment, the disease associated with protein misfolding is selected from Alzheimer's disease, Parkinson's disease, Amyotrophic Lateral Sclerosis (ALS), multiple sclerosis, CADASIL Syndrome, stroke, Friedreich's ataxia, cerebellar ataxia, spinocerebellar ataxia, dementia, prion-caused diseases, Lewy body diseases, amyloidosis, spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia, spasmodic torticollis and other dyskinesis, familial tremor, Gilles de la Tourette syndrome, cerebellar cortical degeneration, spinocerebellar degeneration, Shy-Drager syndrome, spinal muscular atrophy, primary lateral sclerosis, hereditary spastic paraplegia, peroneal muscular atrophy (Charcot-Marie-Tooth), hypertrophic interstitial polyneuropathy (Dejerine-Sottas), nervous system tumors, and secondary neurodegeneration. In one embodiment, the nervous system tumors are selected from the group consisting of astrocytoma, gliomas, glioblastoma, ependymoma, medulloblastoma, CNS lymphoma, craniopharyngioma, glioblastoma, meningioma, pituitary carcinomas, pituitary adenoma, neurofibromatosis, embryonal tumors, tumors of the pineal region, tumors of meninges, choroid plexus tumors, neuronal and mixed neuronal-glial tumors, and germ cell tumors. In one embodiment, the secondary neurodegeneration is selected from the group consisting of neurodegeneration resulting from destruction of neurons by neoplasm, edema, hemorrhage, stroke, trauma, immune attack, hypoxia, poisoning, metabolic defects, and infections.

In another aspect, the invention provides a method for treating a neurodegenerative, neurodevelopmental, psychiatric, autoimmune or oncological disease or an infection associated with protein misfolding in a subject in need thereof, said method comprising administering to the subject a therapeutically effective amount of any of the above vectors or compositions.

In a further aspect, the invention provides a method for delivering an enzyme which has a deoxyribonuclease (DNase) activity to the nervous system in a subject in need thereof, comprising administering to the subject an expression vector comprising a nucleic acid comprising a promoter operably linked to a sequence encoding the enzyme, wherein the promoter is a nervous system-specific promoter.

In a further aspect, the invention provides a method for treating a disease associated with protein misfolding in a subject in need thereof, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter. In one embodiment, the protein misfolding is associated with the presence of cell-free DNA (cfDNA) in cerebrospinal fluid (C SF) or nervous system tissue(s). In one embodiment, the cfDNA is microbial and/or viral cfDNA. In one embodiment, the microbial cfDNA is bacterial cfDNA. In one embodiment, the viral cfDNA is bacteriophage cfDNA. In one embodiment, the disease associated with protein misfolding is selected from neurodegenerative diseases, neurodevelopmental diseases, psychiatric diseases, autoimmune diseases, oncological diseases and infections. In one embodiment, the disease associated with protein misfolding is selected from Alzheimer's disease, Parkinson's disease, Amyotrophic Lateral Sclerosis (ALS), multiple sclerosis, CADASIL Syndrome, stroke, Friedreich's ataxia, cerebellar ataxia, spinocerebellar ataxia, dementia, prion-caused diseases, Lewy body diseases, amyloidosis, spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia, spasmodic torticollis and other dyskinesis, familial tremor, Gilles de la Tourette syndrome, cerebellar cortical degeneration, spinocerebellar degeneration, Shy-Drager syndrome, spinal muscular atrophy, primary lateral sclerosis, hereditary spastic paraplegia, peroneal muscular atrophy (Charcot-Marie-Tooth), hypertrophic interstitial polyneuropathy (Dejerine-Sottas), nervous system tumors, and secondary neurodegeneration. In one embodiment, the nervous system tumors are selected from the group consisting of astrocytoma, gliomas, glioblastoma, ependymoma, medulloblastoma, CNS lymphoma, craniopharyngioma, glioblastoma, meningioma, pituitary carcinomas, pituitary adenoma, neurofibromatosis, embryonal tumors, tumors of the pineal region, tumors of meninges, choroid plexus tumors, neuronal and mixed neuronal-glial tumors, and germ cell tumors. In one embodiment, the secondary neurodegeneration is selected from the group consisting of neurodegeneration resulting from destruction of neurons by neoplasm, edema, hemorrhage, stroke, trauma, immune attack, hypoxia, poisoning, metabolic defects, and infections.

In a further aspect, the invention provides a method for treating a neurodegenerative, neurodevelopmental, psychiatric, autoimmune or oncological disease or an infection in a subject in need thereof, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter.

In one embodiment of any of the above methods, the vector further comprises one or more molecules capable of targeting of the nucleic acid to nervous system.

In one embodiment of any of the above methods, the nervous system-specific promoter mediates a substantially increased expression of the enzyme in the nervous system as compared to other tissues and organs.

Non-limiting examples of the neurodegenerative disease treatable by the methods of the invention include, e.g., Alzheimer's disease, Parkinson's disease, Amyotrophic Lateral Sclerosis (ALS), multiple sclerosis, CADASIL Syndrome, Friedreich's ataxia, cerebellar ataxia, spinocerebellar ataxia, dementia (e.g., fronto-temporal dementia, frontotemporal dementia with parkinsonism-17 (FTDP-17), familial Danish dementia, and familial British dementia), prion-caused diseases, Lewy body diseases, an amyloidosis (e.g., hereditary cerebral hemorrhage with amyloidosis, senile systemic amyloidosis, an amyloidosis with a hereditary cerebral hemorrhage, a primary systemic amyloidosis, a secondary systemic amyloidosis, a serum amyloidosis, a senile systemic amyloidosis, a hemodialysis-related amyloidosis, a Finnish hereditary systemic amyloidosis, an Atrial amyloidosis, a Lysozyme systemic amyloidosis, an Insulin-related amyloidosis, or a Fibrinogen α-chain amyloidosis), Spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm or dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia), Shy-Drager syndrome, spinal muscular atrophy, primary lateral sclerosis, hereditary spastic paraplegia, peroneal muscular atrophy (Charcot-Marie-Tooth), and hypertrophic interstitial polyneuropathy (Dejerine-Sottas). In one embodiment, the neurodegenerative disease is secondary to diabetes, rheumatoid arthritis, systemic lupus erythematosus (SLE), gout, metabolic syndrome, an amyloidosis, asthma, or prion disease.

Non-limiting examples of neurodevelopmental disease treatable by the methods of the invention include, e.g., autism, neural tube defects, attention deficit hyperactivity disorder, Dawn syndrome, cerebral palsy, Rett syndrome, Landau-Kleffner syndrome, Alexander disease, Alpers' disease, alternating hemiplegia, Angelman syndrome, ataxias and cerebellar or spinocerebellar degeneration, ataxia telangiectasia, attention deficit-hyperactivity disorder, autism spectrum disorders, Asperger syndrome, Batten disease, Canavan disease, Tourette syndrome, and impairments in vision and/or hearing.

Non-limiting examples of onclological diseases treatable by the methods of the invention include, e.g., astrocytoma, gliomas, glioblastoma, ependymoma, medulloblastoma, central nervous system (CNS) lymphoma, craniopharyngioma, glioblastoma, meningioma, pituitary carcinomas, pituitary adenomas, neurofibromatosis, embryonal tumors, tumors of the pineal region, tumors of meninges, choroid plexus tumors, neuronal and mixed neuronal-glial tumors, germ cell tumors, anaplastic astrocytoma, central neurocytoma, choroid plexus carcinoma, choroid plexus papilloma, dysembryoplastic neuroepithelial tumor, giant-cell glioblastoma, gliosarcoma, hemangiopericytoma, medulloepithelioma, neuroblastoma, neurocytoma, oligoastrocytoma, oligodendroglioma, optic nerve sheath meningioma, pilocytic astrocytoma, pinealoblastoma, pineocytoma, pleomorphic anaplastic neuroblastoma, pleomorphic xanthoastrocytoma, sphenoid wing meningioma, subependymal giant cell astrocytoma, subependymoma, trilateral retinoblastoma, and nervous system metastasis of any origin.

Non-limiting examples of autoimmune diseases treatable by the methods of the invention include, e.g., autoimmune encephalitis, autoimmune-related epilepsy, central nervous system (CNS) vasculitis, Hashimoto's encephalopathy, steroid-responsive encephalopathy, neurosarcoidosis, Neuro-Behcet's disease, cerebral lupus, neuromyelitis optica, optic neuritis, diabetes, rheumatoid arthritis, systemic lupus erythematosus (SLE), gout, asthma, celiac disease, ankylosing spondylitis, vasculitis, pemphigus vulgaris, inflammatory bowel disease (IBD), Crohn's disease, and ulcerative colitis.

Non-limiting examples of psychiatric diseases treatable by the methods of the invention include, e.g., anxiety disorders, psychotic disorders, schizophrenia, bipolar disorder, depression, post-traumatic stress disorder, and epilepsy.

Non-limiting examples of infections treatable by the methods of the invention include, e.g., adenoviral infections, hepatitis B, hepatitis G, poxvirus infections, herpesvirus infections, papillomavirus infections, HIV infections, meningitis (bacterial, fungal, or viral), bacterial persistence in brain, fungal persistence in brain, pancreatitis, and peritonitis.

In one embodiment of any of the above methods, the subject is human.

In one aspect, the invention provides a recombinant adeno-associated virus (rAAV) expression vector comprising (i) a capsid protein and (ii) a nucleic acid comprising a promoter operably linked to a nucleotide sequence encoding an enzyme which has a deoxyribonuclease (DNase) activity, wherein the promoter is a nervous system-specific promoter. In one embodiment, the nervous system-specific promoter mediates a substantially increased expression of the enzyme in the nervous system tissues and/or CSF as compared to other bodily fluids, tissues and organs.

In one embodiment, the nervous system-specific promoter that target, e.g., glial cells (oligodendrocytes, astrocytes, ependymal cells, microglia, Schwann cells, satellite cells), myeloid tissues, neurons, or other neural cell of peripheral nervous system. Specific non-limiting examples of nervous system-specific promoters and control elements include, microglia-specific promoters (e.g., F4/80, CD68, TMEM119, CX3CR1, CMV, and Iba1 promoters), myeloid-specific promoters (e.g., TTR, CD11b, and c-fes promoters), neuron-specific promoters (e.g., CMV, NSE, synapsin [e.g., SynI, SynII], CamKII , GfaABC1D-dYFP, α-CaMKII, and VGLUT1 promoters), and other neural and glial cell (e.g., oligodendrocytes astrocytes) type-specific promoters (e.g., glial fibrillary acidic protein [GFAP] promoter). In some embodiments, the nervous system-specific promoter is a central nervous system (CNS)-specific promoter. In other embodiments, the nervous system-specific promoter is the enteric nervous system (ENS)-specific promoter.

In another aspect, the invention provides a recombinant adeno-associated virus expression vector (rAAV) comprising (i) a capsid protein and (ii) a nucleic acid comprising a promoter operably linked to a nucleotide sequence encoding an enzyme which has a deoxyribonuclease (DNase) activity, wherein the capsid protein mediates efficient and/or preferential targeting of the vector to the CSF when administered in vivo. In one embodiment, the capsid protein is VP3. In one embodiment, the capsid protein comprises one or more mutations which improve efficiency and/or specificity of the delivery of the vector to the nervous system as compared to the corresponding wild-type capsid protein. In one specific embodiment, the improved efficiency and/or specificity of the delivery of the vector to the nervous system results in a substantially increased expression of the enzyme in the nervous system as compared to other tissues and organs.

Non-limiting examples of enzymes having DNase activity which can be used in the vectors of the invention include, e.g., DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), phosphodiesterase I, lactoferrin, acetylcholinesterase, or mutants or derivatives thereof. In one embodiment, the enzyme which has a DNase activity is a DNase I (e.g., human DNase I) or a mutant or derivative thereof In one embodiment, the DNase I mutant comprises one or more mutations in an actin binding site (e.g., Gln-9, Glu-13, Thr-14, His-44, Asp-53, Tyr-65, Val-66, Val-67, Glu-69, Asn-74, Ala-114, and any combinations thereof; positions indicated in relation to a mature protein sequence lacking secretory signal sequence). In one embodiment, one of the mutations in the actin-binding site is a mutation at Ala-114. In one embodiment, the DNase I mutant comprises one or more mutations increasing DNase activity (e.g., Q9R, E13R, E13K, T14R, T14K, H44R, H44K, N74K, A114F, and any combinations thereof; positions indicated in relation to a mature protein sequence lacking secretory signal sequence). In one embodiment, the DNase I mutant comprises one or more mutations are selected from the group consisting of Q9R, E13R, N74K and A114F, and any combinations thereof. In one embodiment, the DNase I mutant comprises the mutation Q9R. In one embodiment, the DNase I mutant comprises the mutation E13R. In one embodiment, the DNase I mutant comprises the mutation N74K. In one embodiment, the DNase I mutant comprises the mutation A114F.

In one embodiment, the DNase I mutant comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 5. In one embodiment, the DNaseI mutant comprises the mutations Q9R, E13R N74K and A114F. In one embodiment, the DNase I mutant comprises the sequence of SEQ ID NO: 5.

In one embodiment, the DNase I mutant comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 2. In one embodiment, the DNaseI mutant comprises the mutations Q9R, E13R N74K and A114F. In one embodiment, the DNase I mutant comprises the sequence of SEQ ID NO: 2.

In one embodiment, the DNase I mutant consists of the sequence of SEQ ID NO: 2 or SEQ ID NO: 5.

In one embodiment, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P70T, S94N:Y96S, S94N:Y96T, and any combinations thereof. In one embodiment, the nucleotide sequence encodes a DNase I comprising the sequence SEQ ID NO: 4. In one embodiment, the nucleic acid comprises a nucleotide sequence which is at least 85% identical to SEQ ID NO: 23. In one embodiment, the nucleic acid comprises a nucleotide sequence which is at least 90% identical to SEQ ID NO: 23. In one embodiment, the nucleic acid comprises a nucleotide sequence which is at least 95% identical to SEQ ID NO: 23. In one embodiment, the nucleic acid comprises the nucleotide sequence SEQ ID NO: 23. In one embodiment, the nucleotide sequence encodes a DNase I comprising the sequence SEQ ID NO: 1. In one embodiment, the nucleic acid comprises a nucleotide sequence which is at least 85% identical to SEQ ID NO: 22. In one embodiment, the nucleic acid comprises a nucleotide sequence which is at least 90% identical to SEQ ID NO: 22. In one embodiment, the nucleic acid comprises a nucleotide sequence which is at least 95% identical to SEQ ID NO: 22. In one embodiment, the nucleic acid comprises the nucleotide sequence SEQ ID NO: 22. In one embodiment, the nucleic acid comprises a nucleotide sequence which is at least 85% identical to SEQ ID NO: 32. In one embodiment, the nucleic acid comprises a nucleotide sequence which is at least 90% identical to SEQ ID NO: 32. In one embodiment, the nucleic acid comprises a nucleotide sequence which is at least 95% identical to SEQ ID NO: 32. In one embodiment, the nucleic acid comprises the nucleotide sequence SEQ ID NO: 32. In one embodiment, the nucleotide sequence encodes a DNase I mutant comprising the sequence SEQ ID NO: 24. In one embodiment, the nucleic acid comprises a nucleotide sequence which is at least 85% identical to SEQ ID NO: 29. In one embodiment, the nucleic acid comprises a nucleotide sequence which is at least 90% identical to SEQ ID NO: 29. In one embodiment, the nucleic acid comprises a nucleotide sequence which is at least 95% identical to SEQ ID NO: 29. In one embodiment, the nucleic acid comprises the nucleotide sequence SEQ ID NO: 29. In one embodiment, the nucleotide sequence encodes a DNase I mutant comprising the sequence SEQ ID NO: 26. In one embodiment, the nucleic acid comprises a nucleotide sequence which is at least 85% identical to SEQ ID NO: 28. In one embodiment, the nucleic acid comprises a nucleotide sequence which is at least 90% identical to SEQ ID NO: 28. In one embodiment, the nucleic acid comprises a nucleotide sequence which is at least 95% identical to SEQ ID NO: 28. In one embodiment, the nucleic acid comprises the nucleotide sequence SEQ ID NO: 28. In one embodiment, the nucleotide sequence encodes a DNase I mutant comprising the sequence SEQ ID NO: 5. In one embodiment, the nucleic acid comprises a nucleotide sequence which is at least 85% identical to SEQ ID NO: 21. In one embodiment, the nucleic acid comprises a nucleotide sequence which is at least 90% identical to SEQ ID NO: 21. In one embodiment, the nucleic acid comprises a nucleotide sequence which is at least 95% identical to SEQ ID NO: 21. In one embodiment, the nucleic acid comprises the nucleotide sequence SEQ ID NO: 21. In one embodiment, the nucleotide sequence encodes a DNase I mutant comprising the sequence SEQ ID NO: 2. In one embodiment, the nucleic acid comprises a nucleotide sequence which is at least 85% identical to SEQ ID NO: 19. In one embodiment, the nucleic acid comprises a nucleotide sequence which is at least 90% identical to SEQ ID NO: 19. In one embodiment, the nucleic acid comprises a nucleotide sequence which is at least 95% identical to SEQ ID NO: 19. In one embodiment, the nucleic acid comprises the nucleotide sequence SEQ ID NO: 19.

In one embodiment, the enzyme which has a DNase activity is a fusion protein comprising (i) a DNase enzyme or a fragment thereof linked to (ii) an albumin or an Fc polypeptide or a fragment thereof. In one embodiment, the sequence encoding the enzyme which has a DNase activity comprises a sequence encoding a secretory signal sequence, wherein said secretory signal sequence mediates effective secretion of the enzyme into the CSF upon expression of the vector in the nervous system. Non-limiting examples of useful secretory signal sequences include, e.g., DNase I secretory signal sequence, IL2 secretory signal sequence, the albumin secretory signal sequence, the β-glucuronidase secretory signal sequence, the alkaline protease secretory signal sequence, and the fibronectin secretory signal sequence. In one specific embodiment, the secretory signal sequence comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence MRGMKLLGALLALAALLQGAVS (SEQ ID NO: 6). In one specific embodiment, the secretory signal sequence comprises the sequence MRGMKLLGALLALAALLQGAVS (SEQ ID NO: 6). In one specific embodiment, the secretory signal sequence consists of the sequence MRGMKLLGALLALAALLQGAVS (SEQ ID NO: 6). In one specific embodiment, the secretory signal sequence comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence MYRMQLLSCIALSLALVTNS (SEQ ID NO: 7). In one specific embodiment, the secretory signal sequence comprises the sequence MYRMQLLSCIALSLALVTNS (SEQ ID NO: 7). In one specific embodiment, the secretory signal sequence consists of the sequence MYRMQLLSCIALSLALVTNS (SEQ ID NO: 7). In one specific embodiment, the sequence encoding the secretory signal sequence comprises a nucleotide sequence which is at least 85% identical to SEQ ID NO: 20. In one specific embodiment, the sequence encoding the secretory signal sequence comprises a nucleotide sequence which is at least 90% identical to SEQ ID NO: 20. In one specific embodiment, the sequence encoding the secretory signal sequence comprises a nucleotide sequence which is at least 95% identical to SEQ ID NO: 20. In one specific embodiment, the sequence encoding the secretory signal sequence comprises the nucleotide sequence SEQ ID NO: 20.

In one specific embodiment, the secretory signal sequence comprises the sequence MRYTGLMGTLLTLVNLLQLAGT (SEQ ID NO: 25). In one specific embodiment, the secretory signal sequence consists of the sequence MRYTGLMGTLLTLVNLLQLAGT (SEQ ID NO: 25). In one specific embodiment, the secretory signal sequence comprises a sequence having at least 80% sequence identity to sequence MRYTGLMGTLLTLVNLLQLAGT (SEQ ID NO: 25). In one specific embodiment, the sequence encoding the secretory signal sequence comprises a nucleotide sequence which is at least 85% identical to SEQ ID NO: 27. In one specific embodiment, the sequence encoding the secretory signal sequence comprises a nucleotide sequence which is at least 90% identical to SEQ ID NO: 27. In one specific embodiment, the sequence encoding the secretory signal sequence comprises a nucleotide sequence which is at least 95% identical to SEQ ID NO: 27. In one specific embodiment, the sequence encoding the secretory signal sequence comprises the nucleotide sequence of SEQ ID NO: 27.

Non-limiting examples of nervous system-specific promoters which can be used in the vectors of the invention include, e.g., microglia-specific promoters (e.g., F4/80, CD68, TMEM119, CX3CR1, CMV, and Ibal promoters), myeloid-specific promoters (e.g., TTR, CD11b, and c-fes promoters), neuron-specific promoters (e.g., CMV, NSE, synapsin [e.g., SynI, SynII], CamKII , GfaABC1D-dYFP, α-CaMKII, and VGLUT1 promoters), and other neural and glial cell (e.g., oligodendrocytes astrocytes) type-specific promoters (e.g., glial fibrillary acidic protein [GFAP] promoter). In some embodiments, the nervous system-specific promoter is a central nervous system (CNS)-specific promoter. In other embodiments, the nervous system-specific promoter is the enteric nervous system (ENS)-specific promoter.

In one embodiment, the promoter is a F4/80 promoter. In one specific embodiment, the F4/80 promoter comprises the sequence of SEQ ID NO: 38. In one specific embodiment, the F4/80 promoter consists of the sequence of SEQ ID NO: 38.

In one embodiment, the promoter is an CMV promoter. In one specific embodiment, the CMV promoter comprises a sequence having at least over 80% identity to the sequence of SEQ ID NO: 37. In one specific embodiment, the CMVpromoter comprises the sequence of SEQ ID NO: 37. In one specific embodiment, the CMV promoter consists of the sequence of SEQ ID NO: 37.

In one embodiment, the promoter is a synapsin promoter. In one specific embodiment, the synapsin promoter comprises a sequence having at least over 80% identity to the sequence of SEQ ID NO: 36. In one specific embodiment, the synapsin promoter comprises the sequence of SEQ ID NO: 36. In one specific embodiment, the synapsin promoter consists of the sequence of SEQ ID NO: 36.

In one embodiment, the capsid protein comprises one or more mutations selected from the group consisting of S279A, S671A, K137R, T252A, and any combinations thereof In one embodiment, the one or more mutations in the capsid protein include mutation K137R. In one embodiment, the capsid protein comprises the sequence SEQ ID NO:3 [Anc80]. In one embodiment, the capsid protein consists of the sequence SEQ ID NO:3 [Anc80]. In one embodiment, the capsid protein comprises the sequence SEQ ID NO:9 [Anc80]. In one embodiment, the capsid protein consists of the sequence SEQ ID NO:9 [Anc80]. In one embodiment, the capsid protein comprises the sequence SEQ ID NO:34 [Anc80L65]. In one embodiment, the capsid protein consists of the sequence SEQ ID NO:34 [Anc80L65]. In one embodiment, the capsid protein comprises the sequence SEQ ID NO:35 [Anc80L65 variant]. In one embodiment, the capsid protein consists of the sequence SEQ ID NO:35 [Anc80L65 variant]. In one embodiment, the capsid protein is a mutant AAV8 capsid protein such as, e.g., AAV3G1, AAVT20 or AAVTR1, or another mutant capsid protein disclosed in Int. Pat. Appl. Pub. No. WO2017/180854 (e.g., comprising VP3 mutations in amino acids 263-267 [e.g., 263NGTSG267->SGTH (“NGTSG” disclosed as SEQ ID NO: 47 and “SGTH” disclosed as SEQ ID NO: 48) or 263NGTSG267->SDTH (“NGTSG” disclosed as SEQ ID NO: 47 and “SDTH” disclosed as SEQ ID NO: 49)] and/or amino acids 457-459 [e.g., 457TAN459->SRP], and/or amino acids 455-459 [e.g., 455GGTAN459 ->DGSGL (“GGTAN” disclosed as SEQ ID NO: 50 and “DGSGL” disclosed as SEQ ID NO: 51)] and/or amino acids 583-597).

Non-limiting examples of AAVs which can be used in the vectors of the invention include, e.g., serotype 1 (AAV1), AAV2, AAV3 (including AAV3A and AAV3B), AAV4, AAV5, AAV6, AAV7, AAV8, AAV9, AAV10, AAV11, AAV12, AAVrh10, AAVLK03, AAVLK06, AAVLK12, AAV-KP1, AAV-F, AAVDJ, AAV-PHP.B, AAVhu37, AAVrh64R1, and Anc 80. In one specific embodiment, the AAV is from serotype 8 or Anc 80.

In one embodiment, the nucleic acid of the AAV vector further comprises two AAV inverted terminal repeats (ITRs), wherein the ITRs flank the nucleotide sequence encoding the enzyme which has a DNase activity.

In one embodiment, the nucleic acid of the AAV vector further comprises one or more enhancers located upstream or downstream of the promoter. In one embodiment, the enhancer may be located immediately upstream with the promoter, e.g., where the 3′ end of an enhancer sequence fused directly to the 5′ end of the promoter sequence. Non-limiting examples of useful enhancers include, e.g., nPE2 enhancer, Gal4 enhancer, foxP2 enhancer, Mef2 enhancer, CMV enhancer, and any combination thereof In one specific embodiment, the enhancer is nPE2 enhancer. In one specific embodiment, the enhancer is an nPE2 enhancer located upstream of the promoter. In one specific embodiment, the enhancer is an nPE2 enhancer fused to the 5′ end of the promoter.

In one embodiment, the nucleic acid of the AAV vector further comprises a polyadenylation signal operably linked to the nucleotide sequence encoding the enzyme which has a DNase activity.

In one embodiment, the nucleic acid further comprises a Kozak sequence. In one specific embodiment, the Kozak sequence comprises the sequence of 5′-GCCGCCACC-3′ (SEQ ID NO: 33). In one embodiment, the nucleic acid comprises a nucleotide sequence which is at least 80% identical to SEQ ID NO: 30. In one embodiment, the nucleic acid comprises a nucleotide sequence which is at least 85% identical to SEQ ID NO: 30. In one embodiment, the nucleic acid comprises a nucleotide sequence which is at least 90% identical to SEQ ID NO: 30. In one embodiment, the nucleic acid comprises a nucleotide sequence which is at least 95% identical to SEQ ID NO: 30. In one embodiment, the nucleic acid comprises the nucleotide sequence SEQ ID NO: 30.

In one embodiment, the nucleic acid further comprises a post-transcriptional regulatory element. In one embodiment, the post-transcriptional regulatory element is a woodchuck hepatitis post-transcriptional regulatory element (WPRE). In one specific embodiment, the WPRE does not encode a functional X protein. In one embodiment, the post-transcriptional regulatory element comprises a sequence having over 80%, identity to the sequence of SEQ ID NO: 16. In one specific embodiment, the post-transcriptional regulatory element comprises the sequence of SEQ ID NO: 16. In one specific embodiment, the post-transcriptional regulatory element consists of the sequence of SEQ ID NO: 16.

In one embodiment, the invention provides a recombinant adeno-associated virus (rAAV) expression vector comprising (i) a capsid protein comprising the sequence SEQ ID NO: 34 and (ii) a nucleic acid comprising a nucleotide sequence encoding the deoxyribonuclease (DNase) enzyme comprising the sequence SEQ ID NO: 4 operably linked to a F4/80 promoter or a CMV promoter.

In one embodiment, the invention provides a recombinant adeno-associated virus (rAAV) expression vector comprising (i) a capsid protein comprising the sequence SEQ ID NO: 34 and (ii) a nucleic acid comprising a nucleotide sequence encoding the deoxyribonuclease (DNase) enzyme comprising the sequence SEQ ID NO: 5 operably linked to a F4/80 promoter or a CMV promoter.

In one embodiment, the invention provides a recombinant adeno-associated virus (rAAV) expression vector comprising (i) a capsid protein comprising the sequence SEQ ID NO: 34 and (ii) a nucleic acid comprising a nucleotide sequence encoding the deoxyribonuclease (DNase) enzyme comprising the sequence SEQ ID NO: 1 operably linked to a F4/80 promoter or a CMV promoter.

In one embodiment, the invention provides a recombinant adeno-associated virus (rAAV) expression vector comprising (i) a capsid protein comprising the sequence SEQ ID NO: 34 and (ii) a nucleic acid comprising a nucleotide sequence encoding the deoxyribonuclease (DNase) enzyme comprising the sequence SEQ ID NO: 2 operably linked to a F4/80 promoter or a CMV promoter.

In one embodiment, the invention provides a recombinant adeno-associated virus (rAAV) expression vector comprising (i) a capsid protein comprising the sequence SEQ ID NO: 34 and (ii) a nucleic acid comprising the nucleotide sequence of SEQ ID NO: 30.

In one embodiment, the invention provides a recombinant adeno-associated virus (rAAV) expression vector comprising (i) a capsid protein comprising the sequence SEQ ID NO: 34 and (ii) a nucleic acid comprising the nucleotide sequence of SEQ ID NO: 31.

In a related aspect, the invention provides pharmaceutical compositions and dosage forms comprising any of the rAAV vectors of the invention and a pharmaceutically acceptable carrier and/or excipient.

In a related aspect, the invention provides a method for delivering an enzyme which has a deoxyribonuclease (DNase) activity to the nervous system in a subject in need thereof, comprising administering to the subject any of the rAAV vectors or pharmaceutical compositions described above.

In another aspect, the invention provides a method for treating a disease or condition (e.g., neurodegenerative diseases, nervous system tumors, autoimmune diseases, neurodevelopmental diseases) in a subject in need thereof, wherein the disease or condition is associated with the protein aggregation due to a seeding activity of microbial (e.g., bacterial, fungal, intracellular or extracellular parasites) and/or viral (e.g., bacteriophages, eukaryotic viruses) cfDNA within CNS of the subject (with or without the elevation of cfDNA level in bodily fluids), wherein said method comprising administering to the subject a therapeutically effective amount of any of the rAAV vectors or pharmaceutical compositions described above.

In another aspect, the invention provides a method for treating a neurodegenerative, neurodevelopmental, autoimmune or oncological (e.g., nervous system tumor) disease in a subject in need thereof, said method comprising administering to the subject a therapeutically effective amount of any of the rAAV vectors or pharmaceutical compositions described above. In one embodiment, the disease is associated with the formation of a misfolded protein due to the presence of microbial (e.g., bacterial, fungal, intracellular or extracellular parasites) and/or viral (e.g., bacteriophages, eukaryotic visruses) cfDNA without the elevation of cfDNA level in CSF and nervous tissues . In another embodiment, the disease is associated with the formation of a misfolded protein due to the presence of microbial and/or viral cfDNA with the elevation of cfDNA level in bodily fluids.

In one embodiment, the nervous system-specific rAAV vector or the vector composition is administered before, during or after a treatment of the diseases. Non-limiting examples of such treatments for neurodegenerative diseases include transcranial magnetic stimulation, transcranial direct current stimulation, administration of one or more compounds selected from the group consisting of Crenezumab, Solanezumab, CAD106, non-steroidal anti-inflammatory drugs, caffein A2A receptor antagonists, CERE-120 (adeno-associated virus serotype 2-neurturin), levodopa, amantadine, donepezil, hidergine, benztropine, biperiden, bromocriptine, carbidopa, entacapone, edaravone, etanercept. entacapone, galanthamine, laquinimod, memantine, pramipexole, peroglide, pramipexole pramiperoxole, prodopidine, procyclidine, rasagiline , riluzole, radicava, rivastigmine, ropinirole, rotigotine, selegiline, tacrine, tetrabenazine, tolcapone, trihexyphenidyl, gantenerumab, solanezumab, vitamin E, cognitive-enhancing agents, drugs for behavioral symptoms, disease-modifying therapies, drugs targeting amyloid-related mechanisms, drugs targeting tau-related mechanisms, histone acetyltransferase activators, cyclin-dependent protein kinase 5 inhibitors, neurotrophin mimetics, semaphorin-4D blockers, microsomal prostaglandin E synthase-1 inhibitors, N-methyl-d-aspartate (NMDA) receptor inhibitors, anti-amyloid drugs, neurotransmitter based drugs, gene silencing therapies, gene editing therapies, anti-inflammatory therapies, therapies using immunomodulators, axon regeneration, neuroprotection, sigma-1 receptor (S1R) and/or muscarinic receptor agonists, proteostasis, protein misfolding modulators, neuroprotection, anitoxidants, SOD1-lowering therapy, SOD1 Metalation, mitochondrial bioenergetics, nuclear export blockers, misfolded proteins clearance, unfolded protein response (UPR), IRE1 alpha inhibitors, immunotherapy (with different mechanisms of action, including but not limited to monoclonal antibody, polyclonal antibody, etc.), mast cell stabilizers, sigma 1 receptor agonists, NMDA receptor antagonists, TNF alfa inhibitors, BACE1 inhibitors, 5-HT2A antagonists, dopamine receptor modulators, inhibitors of protein aggregation, modulators of GABA-A receptors.

In another aspect, the invention provides a method for preventing or ameliorating one or more side effects associated with a chemotherapy or a radiation therapy (including surgery using radiation) of nervous system tumors in a subject in need thereof, said method comprising administering to the subject a therapeutically effective amount of any of AAV vectors or pharmaceutical compositions described above. In one embodiment, said one or more side effects of the chemotherapy are selected from the group consisting of neurodegeneration, catabolic changes in CSF, fatigue, nausea, vomiting, fibrosis, depression. In one embodiment, the chemotherapy comprises administration of one or more compounds with a non-limiting example of being selected from the group consisting of anthracycline, taxane, 5-fluorouracil, everolimus, bevacizumab, doxorubicin, etoposide, BiCNU, carmustine, lomustine, temozolomide.

In one embodiment, said one or more side effects of the radiation therapy are selected from the group consisting of neurodegeneration, fatigue, nausea, catabolic changes in CSF, vomiting, fibrosis, depression, and a second cancer. In one embodiment, the radiation therapy is external beam radiation therapy or systemic radioisotope therapy.

In one embodiment, the nervous system-specific rAAV vector or the vector composition is administered before, during or after a cycle of the chemotherapy or radiation therapy.

In one embodiment of any of the above methods of the invention, the administration of the rAAV vector or the vector composition results in the expression of the enzyme which has a DNase activity and its secretion into CNS circulation (CSF) of the subject.

In one embodiment of any of the above methods of the invention, the rAAV vector or the vector composition is administered in a dose and regimen which is sufficient to prevent protein misfolding in nervous system (e.g., CSF) of said subject.

In one embodiment of any of the above methods of the invention, the subject is human.

In another aspect, the invention provides a method for delivering an enzyme which has a deoxyribonuclease (DNase) activity to the nervous system in a subject in need thereof, comprising administering to the subject an expression vector comprising a nucleic acid comprising a promoter operably linked to a sequence encoding the enzyme, wherein the promoter is a nervous system-specific promoter.

In a further aspect, the invention provides a method for delivering an enzyme which has a deoxyribonuclease (DNase) activity to the nervous system in a subject in need thereof, comprising administering to the subject an expression vector comprising a nucleic acid comprising a promoter operably linked to a sequence encoding the enzyme, wherein the vector comprises one or more molecules capable of targeting the nucleic acid in nervous system (e.g. CSF) when administered in vivo.

In yet another aspect, the invention provides a method for treating a disease or condition in a subject in need thereof, wherein the disease or condition is accompanied by elevated levels of cfDNA in the CSF circulation of the subject, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter. In one embodiment, the disease is selected from the group consisting of diseases associated with protein misfolding such as neurodevelopmental, neurodegenerative, autoimmune diseases and oncological diseases of the nervous system (including nervous system tumors and nervous system metastasis of any origin).

In yet another aspect, the invention provides a method for treating a disease or condition in a subject in need thereof, wherein the disease or condition is characterized by normal (not elevated) levels of cfDNA in the CSF circulation of the subject, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter. In one embodiment, the disease is selected from the group consisting of diseases associated with protein misfolding such as neurodevelopmental, neurodegenerative, autoimmune diseases, and oncological diseases of the nervous system (including nervous system tumors and nervous system metastasis of any origin).

In another aspect, the invention provides a method for treating a disease or condition in a subject in need thereof, wherein the disease or condition is accompanied by either elevated or not elevated levels of cfDNA in circulation of the subject, said method comprising administering to the subject an expression vector comprising a nucleic acid comprising a promoter operably linked to a sequence encoding an enzyme which has a deoxyribonuclease (DNase) activity, wherein the vector comprises one or more molecules capable of targeting of the nucleic acid to cells of nervous system. In one embodiment, the disease is selected from the group consisting of diseases associated with protein misfolding, including neurodevelopmental, neurodegenerative, autoimmune diseases and oncological diseases of the nervous system (including nervous system tumors and nervous system metastasis of any origin).

In a further aspect, the invention provides a method for treating a cancer in a subject in need thereof, said method comprising administering to the subject an expression vector comprising a nucleic acid encoding an enzyme which has a deoxyribonuclease (DNase) activity, wherein said expression vector provides synthesis of the DNase enzyme in the nervous system of said subject and wherein the cancer originates or is a metastasis to tissues and/or structures of nervous system.

In one embodiment of any of the above methods for treating cancer, the method is effective to inhibit metastasis.

In yet another aspect, the invention provides a method for treating a neurodegenerative, neurodevelopmental, autoimmune, or oncological disease in a subject in need thereof, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter.

In a further aspect, the invention provides a method for treating a neurodegenerative, neurodevelopmental, autoimmune, or oncological disease in a subject in need thereof, said method comprising administering to the subject an expression vector comprising a nucleic acid comprising a promoter operably linked to a sequence encoding an enzyme which has a deoxyribonuclease (DNase) activity, wherein the vector comprises one or more molecules capable of targeting of the nucleic acid to nervous system cells.

In another aspect, the invention provides a method for treating a neurodegenerative, neurodevelopmental, autoimmune, or oncological disease in a subject in need thereof, said method comprising administering to the subject an expression vector comprising a nucleic acid encoding an enzyme which has a deoxyribonuclease (DNase) activity, wherein said expression vector provides synthesis of the DNase enzyme in the CNS of said subject and wherein the microbial (e.g., bacterial, fungal, intracellular or extracellular parasites) and/or viral (e.g., bacteriophages, eukaryotic viruses) cfDNA is detectable in the CSF of said subject. In several embodiments, the method comprises detecting the level of microbial and/or viral cfDNA or the ratio of microbial and/or viral cfDNA to the total level of cfDNA in CSF of the subject. Non-limiting examples of methods of detecting microbial and/or viral cfDNA include, e.g., PCR, RT-PCR, next-generation sequencing (NGS) and whole genome sequencing (WGS). In some embodiments, microbial cfDNA can be detected, e.g., using PCR or RT-PCR of 16S ribosomal RNA (rRNA), 16S ribosomal DNA (rDNA) or Intergenic Spacer Region. In some embodiments, determining the total level or relative content of microbial and/or viral cfDNA includes comparing the level of microbial and/or viral cfDNA or the ratio of the microbial and/or viral cfDNA to the total level of cfDNA in CSF of the subject to a control level or ratio (e.g., a predetermined standard or a corresponding level or ratio determined using age-matched healthy subjects).

In another aspect, the invention provides a method for treating and/or preventing neurodegenerative, neurodevelopmental, psychiatric, autoimmune or oncological diseases or infections in a subject in need thereof, said method comprising administering to the subject an expression vector comprising a nucleic acid encoding an enzyme which has a deoxyribonuclease (DNase) activity, wherein said expression vector provides synthesis of the DNase enzyme in the nervous system of said subject and leads to the destruction of cfDNA, wherein the cfDNA comprises microbial (e.g., bacterial, fungal, intracellular or extracellular parasites) or viral (e.g., bacteriophages, eukaryotic viruses) cfDNA, including DNA from the extracellular vesicles (e.g., exosomes and microvesicles) in the CSF of said subject.

Non-limiting examples of neurodegenerative diseases treatable by any of the above methods for treating neurodegenerative diseases include, e.g., Alzheimer's disease, Mild Cognitive Impairment (MCI), CADASIL syndrome, Parkinson's disease, Amyotrophic Lateral Sclerosis, Huntington's disease, supranuclear palsy (PSP), corticobasal degeneration (CBD), agyrophilic grain disease (AGD), Pick's disease (PiD), Frontotemporal dementia with parkinsonism-17 (FTDP-17), prion-caused diseases, frontotemporal dementia (FTD), frontotemporal dementia with parkinsonism-17 (FTDP-17), Lewy body dementia, vascular dementias, chronic traumatic encephalopathy (CTE), multiple system atrophy (MSA), corticobasal degeneration (CBD), agyrophilic grain disease (AGD), olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm; dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia and related disorders), Shy-Drager syndrome, spinal muscular atrophy, primary lateral sclerosis, hereditary spastic paraplegia, peroneal muscular atrophy (Charcot-Marie-Tooth), hypertrophic interstitial polyneuropathy (Dejerine-Sottas), chronic progressive neuropathy, pigmentary degeneration of the retina (retinitis pigmentosa), and hereditary optic atrophy (Leber's disease), secondary neurodegeneration (e.g., destruction of neurons by neoplasm), neurodegenerative diseases secondary to diabetes, rheumatoid arthritis, systemic lupus erythematosus (SLE), gout, metabolic syndrome, asthma, prion disease, edema, hemorrhage, stroke, trauma, immune attack, hypoxia, poisoning, metabolic defects, infections, or an amyloidosis [e.g., an amyloidosis with a hereditary cerebral hemorrhage, a primary systemic amyloidosis, a secondary systemic amyloidosis, a serum amyloidosis, a senile systemic amyloidosis, a hemodialysis-related amyloidosis, a Finnish hereditary systemic amyloidosis, an Atrial amyloidosis, a Lysozyme systemic amyloidosis, an Insulin-related amyloidosis, or a Fibrinogen a-chain amyloidosis]).

Non-limiting examples of neurodevelopmental diseases treatable by any of the above methods for treating neurodevelopmental diseases include, e.g., autism, neural tube defects, attention deficit hyperactivity disorder, Dawn syndrome, cerebral palsy, and impairments in vision and/or hearing.

Non-limiting examples of autoimmune diseases treatable by any of the above methods for treating autoimmune diseases include, e.g., autoimmune encephalitis, autoimmune-related epilepsy, CNS vasculitis, Hashimoto's encephalopathy, neurosarcoidosis, Neuro-Behcet's disease, cerebral lupus, diabetes, rheumatoid arthritis, systemic lupus erythematosus (SLE), gout, and asthma.

Non-limiting examples of oncological diseases treatable by any of the above methods for treating oncological diseases include, e.g., astrocytoma, gliomas, glioblastoma, ependymoma, medulloblastoma, CNS lymphoma, craniopharyngioma, glioblastoma, meningioma, pituitary carcinomas, pituitary adenomas, neurofibromatosis, embryonal tumors, tumors of the pineal region, tumors of meninges, choroid plexus tumors, neuronal and mixed neuronal-glial tumors, germ cell tumors, and a nervous system metastasis of any origin.

In another aspect, the invention provides a method for preventing or ameliorating one or more side effects associated with a chemotherapy or a radiation therapy (including radiosurgery) in a subject in need thereof, said method comprising administering to the subject an expression vector comprising a nucleic acid comprising a promoter operably linked to a sequence encoding an enzyme which has a deoxyribonuclease (DNase) activity, wherein the promoter is a nervous-system-specific promoter.

In one embodiment of any of the above methods for preventing or ameliorating one or more side effects associated with a chemotherapy, said one or more side effects of the chemotherapy are selected from body weight loss, neurotoxicity, dementia, and catabolic changes in CSF biochemistry.

In one embodiment of any of the above methods for preventing or ameliorating one or more side effects associated with a chemotherapy, the chemotherapy comprises administration of one or more compounds selected from anthracycline, taxane, 5-fluorouracil, everolimus, bevacizumab, doxorubicin, etoposide, BiCNU, carmustine, lomustine, temozolomide.

In one embodiment of any of the above methods for preventing or ameliorating one or more side effects associated with a radiation therapy, said one or more side effects of the radiation therapy are selected from body weight loss, headaches, fatigue, nausea, vomiting, troubles with memory and speech, dementia, and a second cancer.

In one embodiment of any of the above methods for preventing or ameliorating one or more side effects associated with a chemotherapy or a radiation therapy, the vector is administered before, during or after a cycle of the chemotherapy, radiosurgery or radiation therapy.

In one embodiment of any of the methods of the invention, the vector further comprises one or more molecules capable of targeting of the nucleic acid to cells of nervous system.

In one embodiment of any of the methods of the invention, the promoter is a nervous system-specific promoter.

In one embodiment of any of the methods of the invention, the promoter is a periphery-nervous system-specific promoter.

In one embodiment of any of the methods of the invention, the vector is packaged in a liposome or a nanoparticle.

In one embodiment of any of the methods of the invention, the vector is used as a naked DNA.

In one embodiment of any of the methods of the invention, the vector is a viral vector. Non-limiting examples of useful viral vectors include, e.g., adeno-associated virus vectors, adenovirus vectors, and retrovirus vectors (e.g., lentivirus vectors).

In one embodiment of any of the methods of the invention, the enzyme which has a DNase activity is selected from the group consisting of DNase I, DNase X, DNase γ, DNase1L1, DNase1L2, DNase1L3, DNase II, DNase IIα, DNase IIβ, Caspase-activated DNase (CAD), Endonuclease G (ENDOG), Granzyme B (GZMB), phosphodiesterase I, lactoferrin, acetylcholinesterase, and mutants or derivatives thereof In one embodiment, the DNase is DNase I (e.g., human DNase I) or a mutant or derivative thereof. In one embodiment, the DNase I mutant comprises one or more mutations in an actin binding site (e.g., Gln-9, Glu-13, Thr-14, His-44, Asp-53, Tyr-65, Val-66, Val-67, Glu-69, Asn-74, Ala-114, and any combinations thereof; positions indicated in relation to a mature protein sequence lacking secretory signal sequence). In one embodiment, one of the mutations in the actin-binding site is a mutation at Ala-114. In one embodiment, the DNase I mutant comprises one or more mutations increasing DNase activity (e.g., Q9R, E13R, E13K, T14R, T14K, H44R, H44K, N74K, A114F, and any combinations thereof; positions indicated in relation to a mature protein sequence lacking secretory signal sequence). In one embodiment, the DNase I mutant comprises one or more mutations are selected from the group consisting of Q9R, E13R, N74K and A114F, and any combinations thereof. In one embodiment, the DNase I mutant comprises the mutation Q9R. In one embodiment, the DNase I mutant comprises the mutation E13R. In one embodiment, the DNase I mutant comprises the mutation N74K. In one embodiment, the DNase I mutant comprises the mutation A114F.

In one embodiment, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P70T, S94N:Y96S, S94N:Y96T, and any combinations thereof. In one embodiment, the DNase I mutant is a long acting form of DNase. In one embodiment, the DNase I comprises the sequence SEQ ID NO: 4. In one embodiment, the DNase I comprises the sequence SEQ ID NO: 1. In one embodiment, the DNase I mutant comprises the sequence SEQ ID NO: 5. In one embodiment, the DNase I mutant comprises the sequence SEQ ID NO: 2.

In one embodiment of any of the methods of the invention, the sequence encoding the enzyme which has a DNase activity comprises a secretory signal sequence, wherein said secretory signal sequence mediates effective secretion of the enzyme in the ventricles and CSF flow upon administration of the vector to the subject. In one embodiment, the secretory signal sequence is selected from the group consisting of DNase I secretory signal sequence, IL2 secretory signal sequence, albumin secretory signal sequence, β-glucuronidase secretory signal sequence, alkaline protease secretory signal sequence, and fibronectin secretory signal sequence. In one specific embodiment, the secretory signal sequence comprises a sequence having at least 80% identity to the sequence MRGMKLLGALLALAALLQGAVS (SEQ ID NO: 6). In one embodiment, the secretory signal sequence comprises the sequence MRGMKLLGALLALAALLQGAVS (SEQ ID NO: 6). In one embodiment, the secretory signal sequence consists of the sequence MRGMKLLGALLALAALLQGAVS (SEQ ID NO: 6). In one specific embodiment, the secretory signal sequence comprises a sequence having at least 80% identity to the sequence MYRMQLLSCIALSLALVTNS (SEQ ID NO: 7). In one embodiment, the secretory signal sequence comprises the sequence MYRMQLLSCIALSLALVTNS (SEQ ID NO: 7). In one specific embodiment, the secretory signal sequence consists of the sequence MYRMQLLSCIALSLALVTNS (SEQ ID NO: 7).

In one embodiment of any of the methods of the invention, the nervous system-specific promoter mediates a substantially increased expression of the enzyme in the CNS as compared to other tissues and organs. Non-limiting examples of nervous system-specific promoters useful in the methods and vectors of the present invention include, e.g., microglia-specific promoters (e.g., F4/80, CD68, TMEM119, CX3CR1, CMV, and Iba1 promoters), myeloid-specific promoters (e.g., TTR, CD11b, and c-fes promoters), neuron-specific promoters (e.g., CMV, NSE, synapsin [e.g., SynI, SynII], CamKII, α-CaMKII, GfaABC1D-dYFP, and VGLUT1 promoters), and other neural and glial cell (e.g., oligodendrocytes astrocytes) type-specific promoters (e.g., glial fibrillary acidic protein [GFAP] promoter). In some embodiments, the nervous system-specific promoter is a central nervous system (CNS)-specific promoter. In other embodiments, the nervous system-specific promoter is the enteric nervous system (ENS)-specific promoter.

In one specific embodiment, the nervous system-specific promoter is a human F4/80 promoter. In one specific embodiment, the F4/80 promoter comprises the sequence of SEQ ID NO: 38. In one specific embodiment, the F4/80promoter comprises the sequence of SEQ ID NO: 38. In one specific embodiment, the F4/80 promoter consists of the sequence of SEQ ID NO: 38. In various embodiments of any of the methods of the invention, the vector comprises an enhancer. Non-limiting examples of useful enhancers include, e.g., nPE2 enhancer, Gal4 enhancer, foxP2 neuron-specific enhancer element, Mef2 microglia-specific enhancer, and CMV enhancer.

In various embodiments of any of the methods of the invention, the vector comprises a Kozak sequence upstream of the DNase coding sequence. The Kozak sequence may have the sequence of 5′-GCCGCCACC-3′ (SEQ ID NO: 33).

In various embodiments of any of the methods of the invention, the vector comprises a post-transcriptional regulatory element, e.g., the WPRE that does not encode a functional X protein. In one specific embodiment, the post-transcriptional regulatory element comprises the sequence of SEQ ID NO: 16. In one specific embodiment, the post-transcriptional regulatory element consists of the sequence of SEQ ID NO: 16.

In one embodiment of any of the methods of the invention, the administration of the vector results in the expression of the enzyme and its secretion into the CSF circulation of the subject.

In one embodiment of any of the methods of the invention, the vector is administered in a dose and regimen which is sufficient to decrease the level of the cfDNA in the CSF of said subject.

In one embodiment of any of the methods of the invention, the subject is human.

In one embodiment of any of the methods of the invention, the method further comprises selecting a subject with an elevated level of cfDNA in the CSF as compared to the level of cfDNA in the CSF of a normal healthy subject matching the case subject by, e.g., age, sex, race, and any combinations thereof.

In one embodiment of any of the methods of the invention, the method further comprises selecting a subject with an elevated level of cfDNA of specific bacteria, fungi or virus (with or without the elevation of the total cfDNA level) in the CSF as compared to the level of the same specific cfDNA in the CSF of a normal healthy subject matching the case subject by, e.g., age, sex, race, and any combinations thereof.

In one embodiment of any of the methods of the invention, the method further comprises selecting a subject with the presence and/or increased levels of specific bacteria, fungi, intracellular or extracellular parasites, bacteriophages, or eukaryotic viruses or their parts in the CSF as compared to the level of the same specific bacteria, fungi, intracellular or extracellular parasites, bacteriophages, or eukaryotic viruses or their parts in the CSF of a normal healthy subject matching the case subject by, e.g., age, sex, race, and any combinations thereof

In one embodiment of any of the methods of the invention, the method further comprises selecting a subject with the presence and/or increased levels of markers of immune alterations (e.g., cytokines, interleukins, immunoglobulins, cells of the immune system) in the CSF as compared to the level of the same markers in the CSF of a normal healthy subject matching the case subject by, e.g., age, sex, race, and any combinations thereof.

In one embodiment of any of the methods of the invention, the method further comprises selecting a subject with the presence of certain specific bacteria, fungi, intracellular or extracellular parasites, bacteriophages, or eukaryotic viruses or their parts in the CSF as compared to a level of cfDNA in the CSF and to CNS of a normal healthy subject matching the case subject by, e.g., age, sex, race, and any combinations thereof.

In one embodiment of any of the methods of the invention, the method further comprises selecting a subject with early stage (pre-disease) neurodegenerative diseases.

In one embodiment of any of the methods of the invention, the method further comprises selecting a subject with genetic predisposition to neurodegenerative diseases or nervous system tumors.

In one embodiment of any of the methods of the invention, the method further comprises selecting a subject with genetic predisposition along with other predispositions (e.g. microbiota-based, immune-based) to the disease at issue.

In one embodiment of any of the methods of the invention, the method further comprises administering a deoxyribonuclease (DNase) enzyme to the subject (e.g., DNase I, DNase X, DNase γ, DNase1L1, DNase1L2, DNase 1L3, DNase II (including DNase IIα and DNase IIβ, Caspase-activated DNase (CAD), Endonuclease G (ENDOG), Granzyme B (GZMB), phosphodiesterase I, lactoferrin, acetylcholinesterase, and mutants or derivatives thereof). In one specific embodiment, the DNase is DNase I or a mutant or derivative thereof.

These and other aspects of the present invention will be apparent to those of ordinary skill in the art in the following description, claims and drawings.

### BRIEF DESCRIPTION OF THE DRAWINGS

FIGS. 1A-1B show the effect of DNA extracted from diverse sources on tau aggregation. To study the effect of DNA on tau aggregation, monomeric tau (22 μM) was incubated with preparations containing 100 ng of DNA extracted from different bacterial species including Pseudomonas aeruginosa (PA), Tetzosporium hominis (TH), Tetzerella alzheimeri (TA), Escherichia coli ATCC 25922 (EC25), Escherichia coli ATCC 472217 (EC47), Porphyromonas gingivalis (PG), Borrelia burgdorferi (BB). Tau was also incubated with same amount of DNA extracted from Candida albicans (CA) and human samples. (FIG. 1A) Tau aggregation was monitored over time by ThT fluorescence. Data corresponds to the average ±standard error of experiments done in triplicate (except for control without seeds that was performed in quintuplicate). (FIG. 1B) The lag phase, estimated as the time in which ThT fluorescence was higher than the threshold of 40 arbitrary units, was calculated for each experiment. The points represent the values obtained in each of the replicates. Data were analyzed by one-way ANOVA, followed by Tukey multiple comparison post-test. *P<0.05; **P<0.01; ***P<0.001.

FIGS. 2A-2B show the Influence of different concentration of E. coli ATCC 25922 DNA on tau aggregation. To study whether the promoting effect of E. coli DNA can be observed at different concentrations of DNA, monomeric tau was incubated under the conditions described above (see FIG. 1) with 1000, 100 and 10 ng of DNA extracted from E. coli ATCC 25922. (FIG. 2A) Tau aggregation was monitored overtime by ThT fluorescence. Data corresponds to the average±standard error of experiments done in triplicate. (FIG. 2B) The lag phase, estimated as the time in which ThT fluorescence was higher than the threshold of 40 arbitrary units, was calculated for each experiment. The points represent the values obtained in each of the replicates. Data were analyzed by one-way ANOVA, followed by Tukey multiple comparison post-test. *P<0.05; **P<0.01; ***P<0.001.

FIGS. 3A-3B show dose-dependent effect of DNA from Porphyromonas gingivalis on tau aggregation. Monomeric tau was incubated under the conditions described above (see FIG. 1) with 1000, 100 and 10 ng of DNA extracted from P. gingivalis. (FIGS. 3A) Tau aggregation was monitored overtime by ThT fluorescence. Data corresponds to the average±standard error of experiments done in triplicate. (FIGS. 3B) The lag phase, estimated as the time in which ThT fluorescence was higher than the threshold of 40 arbitrary units, was calculated for each experiment. The points represent the values obtained in each of the replicates. Data were analyzed by one-way ANOVA, followed by Tukey multiple comparison post-test. *P<0.05; **P<0.01; ***P<0.001.

FIG. 4 shows that transgenic expression of the DNAse I vector with a nervous system-specific promoter significantly inhibits brain tumor growth.

### DETAILED DESCRIPTION OF THE INVENTION

The present invention is based on unexpected discovery that cell-free DNA (cfDNA) of microbial (e.g., bacterial, fungal, intracellular or extracellular parasites) and viral (e.g., bacteriophages, eukaryotic viruses) origin triggers aggregation and misfolding of proteins, including proteins with prion-like properties both intercellulary and extracellularly within nervous system and does so much faster compared to human cfDNA leading to a variety of diseases (e.g., neurodegenerative diseases, neurodevelopmental diseases, psychiatric diseases, autoimmune diseases, oncological diseases, infections, etc.) with or without the increase of the total cfDNA level in CSF. As demonstrated herein, the intracerebrospinal injection of bacterial DNA to 3×TG mice, MitoPark mouse, SOD1-G93A led to significantly aggressive and fast development of Alzheimer's disease, Parkinson's disease and ALS compared to those triggered by the injection of human DNA. Furthermore, as demonstrated herein, microbial and viral cfDNA released within intestine trigger formation of complexes between cfDNA and prion-like proteins of microbial and viral origin. Such complexes are present in intestine, e.g., between apical surface of intestinal epithelial cells and intestinal mucus gel layer. Such complexes can be also detected in blood and in nervous system in mice suffering from neurodegeneration as part of larger complexes of misfolded proteins of neural origin. Both intestinal sub-mucus gel layer zone and intercellular/extracellular nervous tissue space comprise privileged compartments with limited penetration by vast majority of regular components of blood being isolated from blood circulation by so called intestinal and blood-brain barriers.

The present inventors hypothesized that efficient enzymatic cleavage of cfDNA in such privileged compartments likely requires provision of high local concentration of a deoxyribonuclease (DNase) enzyme.

Even high quantities of systemically administered DNase protein show limited efficacy to prevent protein misfolding seeded by microbial and/or viral cfDNA in nervous system due to insufficient permeability of blood-brain barrier (BBB) (Brightman et al., Journal of the Neurological Sciences, 1970, 10(3):215-39). Moreover, unexpectedly, as shown in the Examples section, below, once neurodegeneration is triggered by the direct effect of microbial and/or viral cfDNA on eukaryotic proteins of the nervous system, even transgenic expression of DNase outside the nervous system (e.g., in liver), has a reduced efficacy compared with DNase expressed in the cells of nervous system.

As demonstrated in the Examples section, below, transgenic expression of a DNase I in the nervous system (e.g., using viral expression vectors) leads to almost complete clearance of cfDNA in neural tissues and nervous system circulation when DNase is secreted to ventricular system or CSF. Such transgenic expression of DNase in the nervous system leads to significant anti-neurodegenerative effects and antitumor effects (particularly for the tumors of nervous system), etc. DNase expression within cells of nervous system also provides neuroprotection for the treatment of diseases associated with an increased intestinal and blood-brain barrier permeability, accompanied with microbial and/or viral cfDNA entering CSF and reaching nervous system.

The present invention provides various vectors for delivery of the DNase to the nervous system. Specific non-limiting examples of such vectors include AAV vectors, adenovirus vectors, retrovirus vectors (e.g., lentivirus vectors), nanoparticles (e.g., phosphoramidite nanoparticles), liposomes (e.g., cationic liposomes such as N-[1-(2,3-dioleyloxy)propyl]-N,N,N-trimethyl ammonium chloride (DOTMA), lactoferrin, poly L-lysine, polyethyleneimine, chitosan, etc.).

An enzyme which has a DNase activity may be expressed under the control of a nervous system-specific promoter and/or another nervous system-specific control element (e.g., enhancer). Specific non-limiting examples of nervous system-specific promoters and control elements include, e.g., microglia-specific promoters (e.g., F4/80, CD68, TMEM119, CX3CR1, CMV, and Iba1 promoters), myeloid-specific promoters (e.g., TTR, CD11b, and c-fes promoters), neuron-specific promoters (e.g., CMV, NSE, synapsin [e.g., SynI, SynII], CamKII , α-CaMKII, GfaABC1D-dYFP, and VGLUT1 promoters), and other neural and glial cell (e.g., oligodendrocytes astrocytes) type-specific promoters (e.g., glial fibrillary acidic protein [GFAP] promoter). In some embodiments, the nervous system-specific promoter is a central nervous system (CNS)-specific promoter. In other embodiments, the nervous system-specific promoter is the enteric nervous system (ENS)-specific promoter. The promoter may be upstream or downstream of an enhancer. The promoter sequence may be directly fused to the enhancer sequence.

Specific non-limiting examples of enzymes which have a DNase activity that can be used in the compositions and methods of the invention include DNase I, DNase X, DNase γ, DNase1L1, DNase1L2, DNase 1L3, DNase II (e.g., DNase IIα, DNase IIβ), caspase-activated DNase (CAD), endonuclease G (ENDOG), granzyme B (GZMB), phosphodiesterase I, lactoferrin, acetylcholinesterase, and mutants or derivatives thereof.

If the enzyme which has a DNase activity is DNase I, various mutants weakening actin-binding may be used. Specific non-limiting examples of residues in wild-type recombinant human DNase I (SEQ ID NO: 4) that can be mutated include, e.g., Gln-9, Glu-13, Thr-14, His-44, Asp-53, Tyr-65, Val-66, Val-67, Glu-69, Asn-74, and Ala-114. In various embodiments, the Ala-114 mutation is used. For example, in human DNase I hyperactive mutant comprising the sequence of SEQ ID NO: 5, the Ala-114 residue is mutated. Complementary residues in other DNases may also be mutated. Specific non-limiting examples of mutations in wild-type human recombinant DNAse I include H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P70T, S94N:Y96S, S94N:Y96T.

Various DNase mutants for increasing DNase activity may be used. Specific non-limiting examples of mutations in wild-type human recombinant DNAse I include, e.g., Gln-9, Glu-13, Thr-14, His-44, Asp-53, Tyr-65, Val-66, Val-67, Glu-69, Asn-74, and Ala-114. Specific non-limiting examples of mutations for increasing the activity of wild-type human recombinant DNase I include Q9R, E13R, E13K, T14R, T14K, H44R, H44K, N74K, and A114F. For example, a combination of the Q9R, E13R, N74K and A114F mutations may be used, with such combination found at least in the hyperactive DNase I mutant comprising the sequence of SEQ ID NO: 5.

When AAV vectors are used for DNase expression, they can be derived from any serotype, e.g., from serotype 1 (AAV1), AAV2, AAV3 (e.g., AAV3A, AAV3B), AAV4, AAV5, AAV6, AAV7, AAV8, AAV9, AAV10, AAV11, AAV12, AAVrh10 (as disclosed, e.g., in U.S. Pat. No. 9,790,472, Int. Pat. Appl. Pub. Nos. WO2017/180857 and WO2017/180861), AAVLK03, AAVLK06, AAVLK12 (as disclosed, e.g., in Wang et al., Mol. Ther., 2015, 23(12):1877-1887), AAVhu37 (as disclosed, e.g., in Int. Pat. Appl. Pub. No. WO2017180857), AAVrh64R1 (as disclosed, e.g., in Int. Pat. Appl. Pub. No. WO2017180857), AAV-KP1 (as disclosed, e.g., in Int. Pat. Appl. Pub. No. WO2019191701A1), or Anc80 (Zinn et al., Cell Rep., 2015, 12(67): 1056-1068). In some embodiments, the AAV vectors used for DNase expression are derived from AAV serotypes and mutants which mediate nervous system delivery (e.g., AAV9 or Anc80).

Point mutations can be made to the capsid protein (e.g., VP3) to improve the efficiency and/or specificity of nervous system-specific delivery. Specific non-limiting examples of such point mutations in the AAV8 VP3 capsid protein include, e.g., S279A, S671A, K137R, and T252A, as well as AAV8 capsid mutations disclosed in Int. Pat. Appl. Pub. No. WO2017/180854 (e.g., AAV3G1, AAVT20 or AAVTR1, VP3 mutations in amino acids 263-267 [e.g., 263NGTSG267->SGTH (“NGTSG” disclosed as SEQ ID NO: 47 and “SGTH” disclosed as SEQ ID NO: 48) or 263NGTSG267->SDTH (“NGTSG” disclosed as SEQ ID NO: 47 and “SDTH” disclosed as SEQ ID NO: 49)] and/or amino acids 457-459 [e.g., 457TAN459->SRP], and/or amino acids 455-459 [e.g., 455GGTAN459 ->DGSGL (“GGTAN” disclosed as SEQ ID NO: 50 and “DGSGL” disclosed as SEQ ID NO: 51)] and/or amino acids 583-597).

The vectors and compositions of the invention can be targeted to the nervous system in various modes. Specific non-limiting examples of routes of administration to the nervous system include intranasal, ocular, intravenous, intramuscular, intra-putamen, intraarterial, intracerebral, intracerebroventricular (i.c.v.), cohlear, intracisternal, intraparenchymal injections, intrastriatal, intraspinal, intrathecal, subarachnoid injection.

Sequences

SEQ ID NO: 1-human DNase I, wild-type (WT), precursor; Genbank Accession No.
NP_005214.2; the secretory signal sequence is underlined:
MIRGMKLLGALLALAALLQGAVSLKIAAFNIQTFGETKMSNATLVSYIVQILSRYDIALV
QEVRDSHLTAVGKLLDNLNQDAPDTYHYVVSEPLGRNSYKERYLFVYRPDQVSAVDSY
YYDDGCEPCGNDTFNREPAIVRFFSRFTEVREFAIVPLHAAPGDAVAEIDALYDVYLDV
QEKWGLEDVMLMGDFNAGCSYVRPSQWSSIRLWTSPTFQWLIPDSADTTATPTHCAYD
RIVVAGMLLRGAVVPDSALPFNFQAAYGLSDQLAQAISDHYPVEVMLK
SEQ ID NO: 2-human DNAse I mutant, precursor; the mutated residues as compared to
SEQ ID NO: 1 are in bold and underlined; the secretory signal sequence is underlined:
MRGMKLLGALLALAALLQGAVSLKIAAFNIRTFGRTKMSNATLVSYIVQILSRYDIALV
QEVRDSHLTAVGKLLDNLNQDAPDTYHYVVSEPLGRKSYKERYLFVYRPDQVSAVDS
YYYDDGCEPCGNDTFNREPFIVRFFSRFTEVREFAIVPLHAAPGDAVAEIDALYDVYLDV
QEKWGLEDVMLMGDFNAGCSYVRPSQWSSIRLWTSPTFQWLIPDSADTTATPTHCAYD
RIVVAGMLLRGAVVPDSALPFNFQAAYGLSDQLAQAISDHYPVEVMLK
SEQ ID NO: 3-Anc80 VP1 capsid protein:
AADGYLPDWLEDNLSEGIREWDLKPGAPKPKANQQKQDDGRGLVLPGYKYLGPFNGL
DKGEPVNAADAAALEHDKAYDQQLKAGDNPYLRYNHADAEFQERLQEDTSFGGNLGR
AVFQAKKRVLEPLGLVEEGAKTAPGKKRPVEQSPQEPDSSSGIGKKGQQPAX¹KRLNFG
QTGDSESVPDPQPLGEPPAAPSGVGSNTMX²AGGGAPMADNNEGADGVGNASGNWHC
DSTWLGDRVITTSTRTALPTYNNHLYKQISSQSGX³STNDNTYFGYSTPWGYFDFNRFHC
HFSPRDWQRLINNNWGFRPKX⁴LNFKLFNIQVKEVTTNDGTTTIANNLTSTVQVFTDSEY
QLPYVLGSAHQGCLPPFPADVFMIPQYGYLTLNNGSQAVGRSSFYCLEYFPSQMLRTGN
NFX⁵FSYTFEDVPFHSSYAHSQSLDRLNPLIDQYLYYLSRTQTTSGTAGNRX⁶LQFSQAGP
SSMANQAKNWLPGPCYRQQRVSKTX⁷NQNNNSNFAWTGATKYHLNGRDSLVNPGPA
MATHKDDEDKFFPMSGVLIFGKQGAGNSNVDLDNVMITX⁸EEEIKTTNPVATEX⁹YGTV
ATNLQSX¹⁰NTAPATGTVNSQGALPGMVWQX¹¹RDVYLQGPIWAKIPHTDGHFHPSPLM
GGFGLKEIPPPQILIKNTPVPANPPTTFSPAKFASFITQYSTGQVSVEIEWELQKENSKRWN
PEIQYTSNYNKSTNVDFAVDTNGVYSEPRPIGTRYLTRNL
X¹ = K or R; X² = A or S; X³ = A or G; X⁴ = R or K; X⁵ = E or Q; X⁶ = T or E; X⁷ = A or T;
X⁸ = S or N; X⁹ = Q or E; X¹⁰ = S or A and X¹¹ = N or D.
SEQ ID NO: 4-mature wild-type (WT) human DNase I (without secretory signal sequence;
Genbank Accession No. 4AWN_A:
LKIAAFNIQTFGETKMSNATLVSYIVQILSRYDIALVQEVRDSHLTAVGKLLDNLNQDAP
DTYHYVVSEPLGRNSYKERYLFVYRPDQVSAVDSYYYDDGCEPCGNDTFNREPAIVRFF
SRFTEVREFAIVPLHAAPGDAVAEIDALYDVYLDVQEKWGLEDVMLMGDFNAGCSYV
RPSQWSSIRLWTSPTFQWLIPDSADTTATPTHCAYDRIVVAGMLLRGAVVPDSALPFNF
QAAYGLSDQLAQAISDHYPVEVMLK
SEQ ID NO: 5-mature human DNAse I mutant (without secretory signal sequence); the
mutated residues as compared to SEQ ID NO: 4 are in bold and underlined:
LKIAAFNIRTFGRTKMSNATLVSYIVQILSRYDIALVQEVRDSHLTAVGKLLDNLNQDAP
DTYHYVVSEPLGRKSYKERYLFVYRPDQVSAVDSYYYDDGCEPCGNDTFNREPFIVRFF
SRFTEVREFAIVPLHAAPGDAVAEIDALYDVYLDVQEKWGLEDVMLMGDFNAGCSYV
RPSQWSSIRLWTSPTFQWLIPDSADTTATPTHCAYDRIVVAGMLLRGAVVPDSALPFNF
QAAYGLSDQLAQAISDHYPVEVMLK
SEQ ID NO: 6-secretory signal sequence of human DNase I:
MRGMKLLGALLALAALLQGAVS
SEQ ID NO: 7-secretory signal sequence of IL2:
MYRMQLLSCIALSLALVTNS
SEQ ID NO: 8-human albumin promoter:
ACTAGTTCCAGATGGTAAATATACACAAGGGATTTAGTCAAACAATTTTTTGGCAAG
AATATTATGAATTTTGTAATCGGTTGGCAGCCAATGAAATACAAAGATGAGTCTAGT
TAATAATCTACAATTATTGGTTAAAGAAGTATATTAGTGCTAATTTCCCTCCGTTTGT
CCTAGCTTTTCTCTTCTGTCAACCCCACACGCCTTTGGCACC
SEQ ID NO: 9-Anc80 VP1 capsid protein:
AADGYLPDWLEDNLSEGIREWDLKPGAPKPKANQQKQDDGRGLVLPGYYLGPFNGLD
KGEPVNAADAAALEHDKAYDQQLKAGDNPYLRYNHADAEFQERLQEDTSFGGNLGRA
VFQAKKRVLEPLGLVEEGAKTAPGKKRPVEQSPQEPDSSSGIGKKGQQPAX¹KRLNFGQ
TGDSESVPDPQPLGEPPAAPSGVGSNTMX²AGGGAPADNNEGADGVGNASGNWHCDST
WLGDRVITTSTRTALPTYNNHLYKQISSQSGX³STNDNTYFGYSTPWGYFDFNRFHCHFS
PRDWQRLINNNWGFRPKX⁴LNFKLFNIQVKEVTTNDGTTTIANNLTSTVQVFTDSEYQLP
YVLGSAHQGCLPPFPADVFMIPQYGYLTLNNGSQAVGRSSFYCLEYFPSQMLRTGNNFX⁵
FSYTFEDVPFHSSYAHSQSLDRLNPLIDQYLYYLSRTQTTSGTAGNRX⁶LQFSQAGPSSA
NQAKNWLPGPCYRQQRVSKTX⁷NQNNNSNFAWTGATKYHLNGRDSLVNPGPAMATH
KDDEDKFFPMSGVLIFGKQGAGNSNVDLDNVITX⁸EEEIKTTNPVATEX⁹YGTVATNLQS
X¹⁰NTAPATGTVNSQGALPGVWQX¹¹RDVYLQGPIWAKIPHTDGHFHPSPLMGGFGLKHP
PPQILIKNTPVPANPPTTFSPAKFASFITQYSTGQVSVEIEELQKENSKRWNPEIQYTSNYN
KSTNVDFAVDTNGVYSEPRPIGTRYLTRNL
X¹ = K or R, X² = A or S, X³ = A or G, X⁴ = R or K, X⁵ = E or Q, X⁶ = T or E,
X⁷ = A or T, X⁸ = S or N, X⁹ = Q or E, X¹⁰ = S or A and X¹¹ = N or D
SEQ ID NO: 10-human beta globin primer:
CAACTTCATCCACGTTCACC
SEQ ID NO: 11-forward NLRP3 primer:
GTTCTGAGCTCCAACCATTCT
SEQ ID NO: 12-reverse NLRP3 primer:
CACTGTGGGTCCTTCATCTTT
SEQ ID NO: 13-forward 165 universal bacterial RNA gene primer:
TCGTCGGCAGCGTCAGATGTGTATAAGAGACAGCCTACGGGNGGCWGCAG
SEQ ID NO: 14-reverse 165 universal bacterial RNA gene primer:
GTCTCGTGGGCTCGGAGATGTGTATAAGAGACAGGACTACHVGGGTATCTAATCC
SEQ ID NO: 15-human anti-trypsin promoter sequence:
GATCTTGCTACCAGTGGAACAGCCACTAAGGATTCTGCAGTGAGAGCAGAGGGCCA
GCTAAGTGGTACTCTCCCAGAGACTGTCTGACTCACGCCACCCCCTCCACCTTGGAC
ACAGGACGCTGTGGTTTCTGAGCCAGGTACAATGACTCCTTTCGGTAAGTGCAGTGG
AAGCTGTACACTGCCCAGGCAAAGCGTCCGGGCAGCGTAGGCGGGCGACTCAGATC
CCAGCCAGTGGACTTAGCCCCTGTTTGCTCCTCCGATAACTGGGGTGACCTTGGTTA
ATATTCACCAGCAGCCTCCCCCGTTGCCCCTCTGGATCCACTGCTTAAATACGGACG
AGGACAGGGCCCTGTCTCCTCAGCTTCAGGCACCACCACTGACCTGGGACAGTGAA
T
SEQ ID NO: 16-Woodchuck hepatitis virus post-transcriptional regulatory element that
does not encode functional protein X:
AGTGGCGGCCGCTCGAGCTAGCGGCCGCTCTAGAAGATAATCAACCTCTGGATTAC
AAAATTTGTGAAAGATTGACTGGTATTCTTAACTATGTTGCTCCTTTTACGCTATGTG
GATACGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCAC
GGCGGAACTCATCGCCGCCTGCCTTGCCCGCTGCTGGACAGGGGCTCGGCTGTTGGG
CACTGACAATTCCGTGGTGTTGTCGGGGAAATCATCGTCCTTTCCTTGGCTGCTCGCC
TGTGTTGCCACCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCGCATCGGAC
TAG
SEQ ID NO: 17-apolipoprotein E (ApoE) enhancer, hepatic control region (HCR):
AGGCTCAGAGGCACACAGGAGTTTCTGGGCTCACCCTGCCCCCTTCCAACCCCTCAG
TTCCCATCCTCCAGCAGCTGTTTGTGTGCTGCCTCTGAAGTCCACACTGAACAAACTT
CAGCCTACTCATGTCCCTAAAATGGGCAAACATTGCAAGCAGCAAACAGCAAACAC
ACAGCCCTCCCTGCCTGCTGACCTTGGAGCTGGGGCAGAGGTCAGAGACCTCTCTGG
GCCCATGCCACCTCCAACATCCACTCGACCCCTTGGAATTTCGGTGGAGAGGAGCAG
AGGTTGTCCTGGCGTGGTTTAGGTAGTGTGAGAGG
SEQ ID NO: 18-a polynucleotide encoding human DNase I hyperactive variant of SEQ ID
NO: 5:
ATGAAGCTGCTGGGGGCGCTGCTGGCACTGGCGGCCCTACTGCAGGGGGCCGTGTC
CCTGAAGATCGCAGCCTTCAACATCAGGACATTTGGGAGGACCAAGATGTCCAATG
CCACCCTCGTCAGCTACATTGTGCAGATCCTGAGCCGCTATGACATCGCCCTGGTCC
AGGAGGTCAGAGACAGCCACCTGACTGCCGTGGGGAAGCTGCTGGACAACCTCAAT
CAGGATGCACCAGACACCTATCACTACGTGGTCAGTGAGCCACTGGGACGGAAGAG
CTATAAGGAGCGCTACCTGTTCGTGTACAGGCCTGACCAGGTGTCTGCGGTGGACAG
CTACTACTACGATGATGGCTGCGAGCCCTGCGGGAACGACACCTTCAACCGAGAGC
CATTCATTGTCAGGTTCTTCTCCCGGTTCACAGAGGTCAGGGAGTTTGCCATTGTTCC
CCTGCATGCGGCCCCGGGGGACGCAGTAGCCGAGATCGACGCTCTCTATGACGTCT
ACCTGGATGTCCAAGAGAAATGGGGCTTGGAGGACGTCATGTTGATGGGCGACTTC
AATGCGGGCTGCAGCTATGTGAGACCCTCCCAGTGGTCATCCATCCGCCTGTGGACA
AGCCCCACCTTCCAGTGGCTGATCCCCGACAGCGCTGACACCACAGCTACACCCACG
CACTGTGCCTATGACAGGATCGTGGTTGCAGGGATGCTGCTCCGAGGCGCCGTTGTT
CCCGACTCGGCTCTTCCCTTTAACTTCCAGGCTGCCTATGGCCTGAGTGACCAACTG
GCCCAAGCCATCAGTGACCACTATCCAGTGGAGGTGATGCTGAAGTGA
SEQ ID NO: 19-a polynucleotide encoding human DNAse I mutant precursor of SEQ ID
NO: 2 (secretory signal sequence underlined):
ATGAGGGGCATGAAGCTGCTGGGGGCGCTGCTGGCACTGGCGGCCCTACTGCAGGG
GGCCGTGTCCCTGAAGATCGCAGCCTTCAACATCAGGACATTTGGGAGGACCAAGA
TGTCCAATGCCACCCTCGTCAGCTACATTGTGCAGATCCTGAGCCGCTATGACATCG
CCCTGGTCCAGGAGGTCAGAGACAGCCACCTGACTGCCGTGGGGAAGCTGCTGGAC
AACCTCAATCAGGATGCACCAGACACCTATCACTACGTGGTCAGTGAGCCACTGGG
ACGGAAGAGCTATAAGGAGCGCTACCTGTTCGTGTACAGGCCTGACCAGGTGTCTG
CGGTGGACAGCTACTACTACGATGATGGCTGCGAGCCCTGCGGGAACGACACCTTC
AACCGAGAGCCATTCATTGTCAGGTTCTTCTCCCGGTTCACAGAGGTCAGGGAGTTT
GCCATTGTTCCCCTGCATGCGGCCCCGGGGGACGCAGTAGCCGAGATCGACGCTCTC
TATGACGTCTACCTGGATGTCCAAGAGAAATGGGGCTTGGAGGACGTCATGTTGATG
GGCGACTTCAATGCGGGCTGCAGCTATGTGAGACCCTCCCAGTGGTCATCCATCCGC
CTGTGGACAAGCCCCACCTTCCAGTGGCTGATCCCCGACAGCGCTGACACCACAGCT
ACACCCACGCACTGTGCCTATGACAGGATCGTGGTTGCAGGGATGCTGCTCCGAGG
CGCCGTTGTTCCCGACTCGGCTCTTCCCTTTAACTTCCAGGCTGCCTATGGCCTGAGT
GACCAACTGGCCCAAGCCATCAGTGACCACTATCCAGTGGAGGTGATGCTGAAGTG
A
SEQ ID NO: 20-a polynucleotide encoding the secretory signal sequence (SEQ ID NO: 6)
of human DNase I:
ATGAGGGGCATGAAGCTGCTGGGGGCGCTGCTGGCACTGGCGGCCCTACTGCAGGG
GGCCGTGTCC
SEQ ID NO: 21-a polynucleotide encoding the mature human DNAse I mutant (without
secretory signal sequence) of SEQ ID NO: 5:
CTGAAGATCGCAGCCTTCAACATCAGGACATTTGGGAGGACCAAGATGTCCAATGC
CACCCTCGTCAGCTACATTGTGCAGATCCTGAGCCGCTATGACATCGCCCTGGTCCA
GGAGGTCAGAGACAGCCACCTGACTGCCGTGGGGAAGCTGCTGGACAACCTCAATC
AGGATGCACCAGACACCTATCACTACGTGGTCAGTGAGCCACTGGGACGGAAGAGC
TATAAGGAGCGCTACCTGTTCGTGTACAGGCCTGACCAGGTGTCTGCGGTGGACAGC
TACTACTACGATGATGGCTGCGAGCCCTGCGGGAACGACACCTTCAACCGAGAGCC
ATTCATTGTCAGGTTCTTCTCCCGGTTCACAGAGGTCAGGGAGTTTGCCATTGTTCCC
CTGCATGCGGCCCCGGGGGACGCAGTAGCCGAGATCGACGCTCTCTATGACGTCTA
CCTGGATGTCCAAGAGAAATGGGGCTTGGAGGACGTCATGTTGATGGGCGACTTCA
ATGCGGGCTGCAGCTATGTGAGACCCTCCCAGTGGTCATCCATCCGCCTGTGGACAA
GCCCCACCTTCCAGTGGCTGATCCCCGACAGCGCTGACACCACAGCTACACCCACGC
ACTGTGCCTATGACAGGATCGTGGTTGCAGGGATGCTGCTCCGAGGCGCCGTTGTTC
CCGACTCGGCTCTTCCCTTTAACTTCCAGGCTGCCTATGGCCTGAGTGACCAACTGG
CCCAAGCCATCAGTGACCACTATCCAGTGGAGGTGATGCTGAAGTGA
SEQ ID NO: 22-a polynucleotide encoding human DNase I, wild-type (WT), precursor of
SEQ ID NO: 1:
ATGAGGGGCATGAAGCTGCTGGGGGCGCTGCTGGCACTGGCGGCCCTACTGCAGGG
GGCCGTGTCCCTGAAGATCGCAGCCTTCAACATCCAGACATTTGGGGAGACCAAGA
TGTCCAATGCCACCCTCGTCAGCTACATTGTGCAGATCCTGAGCCGCTATGACATCG
CCCTGGTCCAGGAGGTCAGAGACAGCCACCTGACTGCCGTGGGGAAGCTGCTGGAC
AACCTCAATCAGGATGCACCAGACACCTATCACTACGTGGTCAGTGAGCCACTGGG
ACGGAACAGCTATAAGGAGCGCTACCTGTTCGTGTACAGGCCTGACCAGGTGTCTGC
GGTGGACAGCTACTACTACGATGATGGCTGCGAGCCCTGCGGGAACGACACCTTCA
ACCGAGAGCCAGCCATTGTCAGGTTCTTCTCCCGGTTCACAGAGGTCAGGGAGTTTG
CCATTGTTCCCCTGCATGCGGCCCCGGGGGACGCAGTAGCCGAGATCGACGCTCTCT
ATGACGTCTACCTGGATGTCCAAGAGAAATGGGGCTTGGAGGACGTCATGTTGATG
GGCGACTTCAATGCGGGCTGCAGCTATGTGAGACCCTCCCAGTGGTCATCCATCCGC
CTGTGGACAAGCCCCACCTTCCAGTGGCTGATCCCCGACAGCGCTGACACCACAGCT
ACACCCACGCACTGTGCCTATGACAGGATCGTGGTTGCAGGGATGCTGCTCCGAGG
CGCCGTTGTTCCCGACTCGGCTCTTCCCTTTAACTTCCAGGCTGCCTATGGCCTGAGT
GACCAACTGGCCCAAGCCATCAGTGACCACTATCCAGTGGAGGTGATGCTGAAG
SEQ ID NO: 23-a polynucleotide encoding the mature wild-type (WT) human DNase I of
SEQ ID NO: 4:
CTGAAGATCGCAGCCTTCAACATCCAGACATTTGGGGAGACCAAGATGTCCAATGC
CACCCTCGTCAGCTACATTGTGCAGATCCTGAGCCGCTATGACATCGCCCTGGTCCA
GGAGGTCAGAGACAGCCACCTGACTGCCGTGGGGAAGCTGCTGGACAACCTCAATC
AGGATGCACCAGACACCTATCACTACGTGGTCAGTGAGCCACTGGGACGGAACAGC
TATAAGGAGCGCTACCTGTTCGTGTACAGGCCTGACCAGGTGTCTGCGGTGGACAGC
TACTACTACGATGATGGCTGCGAGCCCTGCGGGAACGACACCTTCAACCGAGAGCC
AGCCATTGTCAGGTTCTTCTCCCGGTTCACAGAGGTCAGGGAGTTTGCCATTGTTCCC
CTGCATGCGGCCCCGGGGGACGCAGTAGCCGAGATCGACGCTCTCTATGACGTCTA
CCTGGATGTCCAAGAGAAATGGGGCTTGGAGGACGTCATGTTGATGGGCGACTTCA
ATGCGGGCTGCAGCTATGTGAGACCCTCCCAGTGGTCATCCATCCGCCTGTGGACAA
GCCCCACCTTCCAGTGGCTGATCCCCGACAGCGCTGACACCACAGCTACACCCACGC
ACTGTGCCTATGACAGGATCGTGGTTGCAGGGATGCTGCTCCGAGGCGCCGTTGTTC
CCGACTCGGCTCTTCCCTTTAACTTCCAGGCTGCCTATGGCCTGAGTGACCAACTGG
CCCAAGCCATCAGTGACCACTATCCAGTGGAGGTGATGCTGAAG
SEQ ID NO: 24-Mus musculus wild type DNase I, precursor; Genbank Accession No.
NP_034191.3; the secretory signal sequence is underlined:
MRYTGLMGTLLTLVNLLQLAGTLRIAAFNIRTFGETKMSNATLSVYFVKILSRYDIAVIQ
EVRDSHLVAVGKLLDELNRDKPDTYRYVVSEPLGRKSYKEQYLFVYRPDQVSILDSYQ
YDDGCEPCGNDTFSREPAIVKFFSPYTEVQEFAIVPLHAAPTEAVSEIDALYDVYLDVWQ
KWGLEDIMFMGDFNAGCSYVTSSQWSSIRLRTSPIFQWLIPDSADTTVTSTHCAYDRIVV
AGALLQAAVVPNSAVPFDFQAEYGLSNQLAEAISDHYPVEVTLRKI
SEQ ID NO: 25-secretory signal sequence of Mus musculus wild type DNase I
MRYTGLMGTLLTLVNLLQLAGT
SEQ ID NO: 26-mature wild-type (WT) Mus musculus wild type DNase I
LRIAAFNIRTFGETKMSNATLSVYFVKILSRYDIAVIQEVRDSHLVAVGKLLDELNRDKP
DTYRYVVSEPLGRKSYKEQYLFVYRPDQVSILDSYQYDDGCEPCGNDTFSREPAIVKFFS
PYTEVQEFAIVPLHAAPTEAVSEIDALYDVYLDVWQKWGLEDIMFMGDFNAGCSYVTS
SQWSSIRLRTSPIFQWLIPDSADTTVTSTHCAYDRIVVAGALLQAAVVPNSAVPFDFQAE
YGLSNQLAEAISDHYPVEVTLRKI
SEQ ID NO: 27-a polynucleotide encoding the secretory signal sequence of Mus musculus
wild type DNase I
ATGCGGTACACAGGGCTAATGGGAACACTGCTCACCTTGGTCAACCTGCTGCAGCTG
GCTGGGACT
SEQ ID NO: 28-a polynucleotide encoding the mature wild-type (WT) Mus musculus
wild type DNase I
CTGAGAATTGCAGCCTTCAACATTCGGACTTTTGGGGAGACTAAGATGTCCAATGCT
ACCCTCTCTGTATACTTTGTGAAAATCCTGAGTCGCTATGACATCGCTGTTATCCAAG
AGGTCAGAGACTCCCACCTGGTTGCTGTTGGGAAGCTCCTGGATGAACTCAATCGGG
ACAAACCTGACACCTACCGCTATGTAGTCAGTGAGCCGCTGGGCCGCAAAAGCTAC
AAGGAACAGTACCTTTTTGTGTACAGGCCTGACCAGGTGTCTATTCTGGACAGCTAT
CAATATGATGATGGCTGTGAACCCTGTGGAAATGACACCTTCAGCAGAGAGCCAGC
CATTGTTAAGTTCTTTTCCCCATACACTGAGGTCCAAGAATTTGCGATCGTGCCCTTG
CATGCAGCCCCAACAGAAGCTGTGAGTGAGATCGACGCCCTCTACGATGTTTACCTA
GATGTCTGGCAAAAGTGGGGCCTGGAGGACATCATGTTCATGGGAGACTTCAATGC
TGGCTGCAGCTACGTCACTTCCTCCCAGTGGTCCTCCATTCGCCTTCGGACAAGCCC
CATCTTCCAGTGGCTGATCCCTGACAGTGCGGACACCACAGTCACATCAACACACTG
TGCTTATGACAGGATTGTGGTTGCTGGAGCTCTGCTCCAGGCTGCTGTTGTTCCCAAC
TCGGCTGTTCCTTTTGATTTCCAAGCAGAATACGGACTTTCCAACCAGCTGGCTGAA
GCCATCAGTGACCATTACCCAGTGGAGGTGACACTCAGAAAAATCTGA
SEQ ID NO: 29-a polynucleotide encoding the Mus musculus wild type DNase I,
precursor
ATGCGGTACACAGGGCTAATGGGAACACTGCTCACCTTGGTCAACCTGCTGCAGCTG
GCTGGGACTCTGAGAATTGCAGCCTTCAACATTCGGACTTTTGGGGAGACTAAGATG
TCCAATGCTACCCTCTCTGTATACTTTGTGAAAATCCTGAGTCGCTATGACATCGCTG
TTATCCAAGAGGTCAGAGACTCCCACCTGGTTGCTGTTGGGAAGCTCCTGGATGAAC
TCAATCGGGACAAACCTGACACCTACCGCTATGTAGTCAGTGAGCCGCTGGGCCGC
AAAAGCTACAAGGAACAGTACCTTTTTGTGTACAGGCCTGACCAGGTGTCTATTCTG
GACAGCTATCAATATGATGATGGCTGTGAACCCTGTGGAAATGACACCTTCAGCAG
AGAGCCAGCCATTGTTAAGTTCTTTTCCCCATACACTGAGGTCCAAGAATTTGCGAT
CGTGCCCTTGCATGCAGCCCCAACAGAAGCTGTGAGTGAGATCGACGCCCTCTACG
ATGTTTACCTAGATGTCTGGCAAAAGTGGGGCCTGGAGGACATCATGTTCATGGGAG
ACTTCAATGCTGGCTGCAGCTACGTCACTTCCTCCCAGTGGTCCTCCATTCGCCTTCG
GACAAGCCCCATCTTCCAGTGGCTGATCCCTGACAGTGCGGACACCACAGTCACATC
AACACACTGTGCTTATGACAGGATTGTGGTTGCTGGAGCTCTGCTCCAGGCTGCTGT
TGTTCCCAACTCGGCTGTTCCTTTTGATTTCCAAGCAGAATACGGACTTTCCAACCAG
CTGGCTGAAGCCATCAGTGACCATTACCCAGTGGAGGTGACACTCAGAAAAATCTG
A
SEQ ID NO: 30-Complete sequence of ApoEHCR enhancer-hAAT promoter-hDNaseI
(hyperactive)correct leader-WPRE Xinact (VR-18013AD)
AGGCTCAGAGGCACACAGGAGTTTCTGGGCTCACCCTGCCCCCTTCCAACCCCTCAG
TTCCCATCCTCCAGCAGCTGTTTGTGTGCTGCCTCTGAAGTCCACACTGAACAAACTT
CAGCCTACTCATGTCCCTAAAATGGGCAAACATTGCAAGCAGCAAACAGCAAACAC
ACAGCCCTCCCTGCCTGCTGACCTTGGAGCTGGGGCAGAGGTCAGAGACCTCTCTGG
GCCCATGCCACCTCCAACATCCACTCGACCCCTTGGAATTTCGGTGGAGAGGAGCAG
AGGTTGTCCTGGCGTGGTTTAGGTAGTGTGAGAGGGATCTTGCTACCAGTGGAACAG
CCACTAAGGATTCTGCAGTGAGAGCAGAGGGCCAGCTAAGTGGTACTCTCCCAGAG
ACTGTCTGACTCACGCCACCCCCTCCACCTTGGACACAGGACGCTGTGGTTTCTGAG
CCAGGTACAATGACTCCTTTCGGTAAGTGCAGTGGAAGCTGTACACTGCCCAGGCAA
AGCGTCCGGGCAGCGTAGGCGGGCGACTCAGATCCCAGCCAGTGGACTTAGCCCCT
GTTTGCTCCTCCGATAACTGGGGTGACCTTGGTTAATATTCACCAGCAGCCTCCCCC
GTTGCCCCTCTGGATCCACTGCTTAAATACGGACGAGGACAGGGCCCTGTCTCCTCA
GCTTCAGGCACCACCACTGACCTGGGACAGTGAATGCCGCCACCATGAGGGGCATG
AAGCTGCTGGGGGCGCTGCTGGCACTGGCGGCCCTACTGCAGGGGGCCGTGTCCCT
GAAGATCGCAGCCTTCAACATCAGGACATTTGGGAGGACCAAGATGTCCAATGCCA
CCCTCGTCAGCTACATTGTGCAGATCCTGAGCCGCTATGACATCGCCCTGGTCCAGG
AGGTCAGAGACAGCCACCTGACTGCCGTGGGGAAGCTGCTGGACAACCTCAATCAG
GATGCACCAGACACCTATCACTACGTGGTCAGTGAGCCACTGGGACGGAAGAGCTA
TAAGGAGCGCTACCTGTTCGTGTACAGGCCTGACCAGGTGTCTGCGGTGGACAGCTA
CTACTACGATGATGGCTGCGAGCCCTGCGGGAACGACACCTTCAACCGAGAGCCAT
TCATTGTCAGGTTCTTCTCCCGGTTCACAGAGGTCAGGGAGTTTGCCATTGTTCCCCT
GCATGCGGCCCCGGGGGACGCAGTAGCCGAGATCGACGCTCTCTATGACGTCTACC
TGGATGTCCAAGAGAAATGGGGCTTGGAGGACGTCATGTTGATGGGCGACTTCAAT
GCGGGCTGCAGCTATGTGAGACCCTCCCAGTGGTCATCCATCCGCCTGTGGACAAGC
CCCACCTTCCAGTGGCTGATCCCCGACAGCGCTGACACCACAGCTACACCCACGCAC
TGTGCCTATGACAGGATCGTGGTTGCAGGGATGCTGCTCCGAGGCGCCGTTGTTCCC
GACTCGGCTCTTCCCTTTAACTTCCAGGCTGCCTATGGCCTGAGTGACCAACTGGCC
CAAGCCATCAGTGACCACTATCCAGTGGAGGTGATGCTGAAGTGAAGTGGCGGCCG
CTCGAGCTAGCGGCCGCTCTAGAAGATAATCAACCTCTGGATTACAAAATTTGTGAA
AGATTGACTGGTATTCTTAACTATGTTGCTCCTTTTACGCTATGTGGATACGCTGCTT
TAATGCCTTTGTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCACC
TGTCAGCTCCTTTCCGGGACTTTCGCTTTCCCCCTCCCTATTGCCACGGCGGAACTCA
TCGCCGCCTGCCTTGCCCGCTGCTGGACAGGGGCTCGGCTGTTGGGCACTGACAATT
CCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGC
CCTCAGACGAGTCGGATCTCCCTTTGGGCCGCCTCCCCGCATCGGACTAG
APOE HCR enhancer: bases 1-320; human alpha-1-antitrypsin promoter: bases 321-717; Kozak
sequence: bases 718-726; human DNaseI hyperactive variant with natural full correct leader
sequence: bases 727-1575; WPRE X protein inactivated: bases 1576-2212
SEQ ID NO: 31-Complete sequence of ApoEHCR enhancer-hAAT promoter-hDNaseI
wild type-WPRE Xinact (VR-18014AD)
AGGCTCAGAGGCACACAGGAGTTTCTGGGCTCACCCTGCCCCCTTCCAACCCCTCAG
TTCCCATCCTCCAGCAGCTGTTTGTGTGCTGCCTCTGAAGTCCACACTGAACAAACTT
CAGCCTACTCATGTCCCTAAAATGGGCAAACATTGCAAGCAGCAAACAGCAAACAC
ACAGCCCTCCCTGCCTGCTGACCTTGGAGCTGGGGCAGAGGTCAGAGACCTCTCTGG
GCCCATGCCACCTCCAACATCCACTCGACCCCTTGGAATTTCGGTGGAGAGGAGCAG
AGGTTGTCCTGGCGTGGTTTAGGTAGTGTGAGAGGGATCTTGCTACCAGTGGAACAG
CCACTAAGGATTCTGCAGTGAGAGCAGAGGGCCAGCTAAGTGGTACTCTCCCAGAG
ACTGTCTGACTCACGCCACCCCCTCCACCTTGGACACAGGACGCTGTGGTTTCTGAG
CCAGGTACAATGACTCCTTTCGGTAAGTGCAGTGGAAGCTGTACACTGCCCAGGCAA
AGCGTCCGGGCAGCGTAGGCGGGCGACTCAGATCCCAGCCAGTGGACTTAGCCCCT
GTTTGCTCCTCCGATAACTGGGGTGACCTTGGTTAATATTCACCAGCAGCCTCCCCC
GTTGCCCCTCTGGATCCACTGCTTAAATACGGACGAGGACAGGGCCCTGTCTCCTCA
GCTTCAGGCACCACCACTGACCTGGGACAGTGAATGCCGCCACCATGAGGGGCATG
AAGCTGCTGGGGGCGCTGCTGGCACTGGCGGCCCTACTGCAGGGGGCCGTGTCCCT
GAAGATCGCAGCCTTCAACATCCAGACATTTGGGGAGACCAAGATGTCCAATGCCA
CCCTCGTCAGCTACATTGTGCAGATCCTGAGCCGCTATGACATCGCCCTGGTCCAGG
AGGTCAGAGACAGCCACCTGACTGCCGTGGGGAAGCTGCTGGACAACCTCAATCAG
GATGCACCAGACACCTATCACTACGTGGTCAGTGAGCCACTGGGACGGAACAGCTA
TAAGGAGCGCTACCTGTTCGTGTACAGGCCTGACCAGGTGTCTGCGGTGGACAGCTA
CTACTACGATGATGGCTGCGAGCCCTGCGGGAACGACACCTTCAACCGAGAGCCAG
CCATTGTCAGGTTCTTCTCCCGGTTCACAGAGGTCAGGGAGTTTGCCATTGTTCCCCT
GCATGCGGCCCCGGGGGACGCAGTAGCCGAGATCGACGCTCTCTATGACGTCTACC
TGGATGTCCAAGAGAAATGGGGCTTGGAGGACGTCATGTTGATGGGCGACTTCAAT
GCGGGCTGCAGCTATGTGAGACCCTCCCAGTGGTCATCCATCCGCCTGTGGACAAGC
CCCACCTTCCAGTGGCTGATCCCCGACAGCGCTGACACCACAGCTACACCCACGCAC
TGTGCCTATGACAGGATCGTGGTTGCAGGGATGCTGCTCCGAGGCGCCGTTGTTCCC
GACTCGGCTCTTCCCTTTAACTTCCAGGCTGCCTATGGCCTGAGTGACCAACTGGCC
CAAGCCATCAGTGACCACTATCCAGTGGAGGTGATGCTGAAGTGAAGTGGCGGCCG
CTCGAGCTAGCGGCCGCTCTAGAAGATAATCAACCTCTGGATTACAAAATTTGTGAA
AGATTGACTGGTATTCTTAACTATGTTGCTCCTTTTACGCTATGTGGATACGCTGCTT
TAATGCCTTTGTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCACC
TGTCAGCTCCTTTCCGGGACTTTCGCTTTCCCCCTCCCTATTGCCACGGCGGAACTCA
TCGCCGCCTGCCTTGCCCGCTGCTGGACAGGGGCTCGGCTGTTGGGCACTGACAATT
CCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGC
CCTCAGACGAGTCGGATCTCCCTTTGGGCCGCCTCCCCGCATCGGACTAG
APOE HCR enhancer: bases 1-320; human alpha-1-antitrypsin promoter: bases 321-717; Kozak
sequence: bases 718-726; human DNaseI wild type with natural full correct leader sequence:
bases 727-1575; WPRE X protein inactivated: bases 1576-2212
SEQ ID NO: 32-a polynucleotide encoding human DNase I, wild-type (WT), precursor of
SEQ ID NO: 1 with stop codon:
ATGAGGGGCATGAAGCTGCTGGGGGCGCTGCTGGCACTGGCGGCCCTACTGCAGGG
GGCCGTGTCCCTGAAGATCGCAGCCTTCAACATCCAGACATTTGGGGAGACCAAGA
TGTCCAATGCCACCCTCGTCAGCTACATTGTGCAGATCCTGAGCCGCTATGACATCG
CCCTGGTCCAGGAGGTCAGAGACAGCCACCTGACTGCCGTGGGGAAGCTGCTGGAC
AACCTCAATCAGGATGCACCAGACACCTATCACTACGTGGTCAGTGAGCCACTGGG
ACGGAACAGCTATAAGGAGCGCTACCTGTTCGTGTACAGGCCTGACCAGGTGTCTGC
GGTGGACAGCTACTACTACGATGATGGCTGCGAGCCCTGCGGGAACGACACCTTCA
ACCGAGAGCCAGCCATTGTCAGGTTCTTCTCCCGGTTCACAGAGGTCAGGGAGTTTG
CCATTGTTCCCCTGCATGCGGCCCCGGGGGACGCAGTAGCCGAGATCGACGCTCTCT
ATGACGTCTACCTGGATGTCCAAGAGAAATGGGGCTTGGAGGACGTCATGTTGATG
GGCGACTTCAATGCGGGCTGCAGCTATGTGAGACCCTCCCAGTGGTCATCCATCCGC
CTGTGGACAAGCCCCACCTTCCAGTGGCTGATCCCCGACAGCGCTGACACCACAGCT
ACACCCACGCACTGTGCCTATGACAGGATCGTGGTTGCAGGGATGCTGCTCCGAGG
CGCCGTTGTTCCCGACTCGGCTCTTCCCTTTAACTTCCAGGCTGCCTATGGCCTGAGT
GACCAACTGGCCCAAGCCATCAGTGACCACTATCCAGTGGAGGTGATGCTGAAGTG
A
SEQ ID NO: 33-Kozak sequence
5′-GCCGCCACC-3′
SEQ ID NO: 34-Anc80L65 VP1 capsid protein
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPLGLVEEGAKTAPGKKRPVEQSPQEPDSSSGIGKKGQQPARKRLN
FGQTGDSESVPDPQPLGEPPAAPSGVGSNTMAAGGGAPMADNNEGADGVGNASGNWH
CDSTWLGDRVITTSTRTWALPTYNNHLYKQISSQSGGSTNDNTYFGYSTPWGYFDFNRF
HCHFSPRDWQRLINNNWGFRPKKLNFKLFNIQVKEVTTNDGTTTIANNLTSTVQVFTDS
EYQLPYVLGSAHQGCLPPFPADVFMIPQYGYLTLNNGSQAVGRSSFYCLEYFPSQMLRT
GNNFQFSYTFEDVPFHSSYAHSQSLDRLMNPLIDQYLYYLSRTQTTSGTAGNRTLQFSQ
AGPSSMANQAKNWLPGPCYRQQRVSKTTNQNNNSNFAWTGATKYHLNGRDSLVNPGP
AMATHKDDEDKFFPMSGVLIFGKQGAGNSNVDLDNVMITNEEEIKTTNPVATEEYGTV
ATNLQSANTAPATGTVNSQGALPGMVWQDRDVYLQGPIWAKIPHTDGHFHPSPLMGG
FGLKHPPPQILIKNTPVPANPPTTFSPAKFASFITQYSTGQVSVEIEWELQKENSKRWNPEI
QYTSNYNKSTNVDFAVDTNGVYSEPRPIGTRYLTRNL
SEQ ID NO: 35-variant Anc80L65 VP1 capsid protein
MAADGYLPDWLEDNLSEGIREWWDLKPGAPKPKANQQKQDDGRGLVLPGYYLGPFN
GLDKGEPVNAADAAALEHDKAYDQQLKAGDNPYLRYNHADAEFQERLQEDTSFGGNL
GRAVFQAKKRVLEPLGLVEEGAKTAPGKKRPVEQSPQEPDSSSGIGKKGQQPARKRLNF
GQTGDSESVPDPQPLGEPPAAPSGVGSNTMAAGGGAPADNNEGADGVGNASGNWHCD
STWLGDRVITTSTRTWALPTYNNHLYKQISSQSGGSTNDNTYFGYSTPWGYFDFNRFHC
HFSPRDWQRLINNNWGFRPKKLNFKLFNIQVKEVTTNDGTTTIANNLTSTVQVFTDSEY
QLPYVLGSAHQGCLPPFPADVFMIPQYGYLTLNNGSQAVGRSSFYCLEYFPSQMLRTGN
NFQFSYTFEDVPFHSSYAHSQSLDRLMNPLIDQYLYYLSRTQTTSGTAGNRTLQFSQAGP
SSANQAKNWLPGPCYRQQRVSKTTNQNNNSNFAWTGATKYHLNGRDSLVNPGPAMA
THKDDEDKFFPMSGVLIFGKQGAGNSNVDLDNVITNEEEIKTTNPVATEEYGTVATNLQ
SANTAPATGTVNSQGALPGVWQDRDVYLQGPIWAKIPHTDGHFHPSPLMGGFGLKEIPP
PQILIKNTPVPANPPTTFSPAKFASFITQYSTGQVSVEIEELQKENSKRWNPEIQYTSNYNK
STNVDFAVDTNGVYSEPRPIGTRYLTRNL
SEQ ID NO: 36-human synapsin promoter
AGTGCAAGTGGGTTTTAGGACCAGGATGAGGCGGGGTGGGGGTGCCTACCTGACGA
CCGACCCCGACCCACTGGACAAGCACCCAACCCCCATTCCCCAAATTGCGCATCCCC
TATCAGAGAGGGGGAGGGGAAACAGGATGCGGCGAGGCGCGTGCGCACTGCCAGC
TTCAGCACCGCGGACAGTGCCTTCGCCCCCGCCTGGCGGCGCGCGCCACCGCCGCCT
CAGCACTGAAGGCGCGCTGACGTCACTCGCCGGTCCCCCGCAAACTCCCCTTCCCGG
CCACCTTGGTCGCGTCCGCGCCGCCGCCGGCCCAGCCGGACCGCACCACGCGAGGC
GCGAGATAGGGGGGCACGGGCGCGACCATCTGCGCTGCGGCGCCGGCGACTCAGCG
CTGCCTCAGTCTGCGGTGGGCAGCGGAGGAGTCGTGTCGTGCCTGAGAGCGCAG
SEQ ID NO: 37-CMV promoter
gcggccgctctagagagcttggcccattgcatacgttgtatccatatcataatatgtacatttatattggctcatgtccaacattaccgccatgttg
acattgattattgactagttattaatagtaatcaattacggggtcattagttcatagcccatatatggagttccgcgttacataacttacggtaaatg
gcccgcctggctgaccgcccaacgacccccgcccattgacgtcaataatgacgtatgttcccatagtaacgccaatagggactttccattga
cgtcaatgggtggagtatttacggtaaactgcccacttggcagtacatcaagtgtatcatatgccaagtacgccccctattgacgtcaatgacg
gtaaatggcccgcctggcattatgcccagtacatgaccttatgggactttcctacttggcagtacatctacgtattagtcatcgctattaccatgg
tgatgcggttttggcagtacatcaatgggcgtggatagcggtttgactcacggggatttccaagtctccaccccattgacgtcaatgggagttt
gttttggcaccaaaatcaacgggactttccaaaatgtcgtaacaactccgccccattgacgcaaatgggcggtaggcgtgtacggtgggag
gtctatataagcagagctcgtttagtgaaccgtcagatcgcctggagacgccatccacgctgttttgacctccatagaagacaccgggaccg
atccagcctccggt
SEQ ID NO: 38-F4/80 promoter
cctctgtctgtctgtctgtctctttggtgtgatgtatgtggtgtgtgtgtattctacaaggttgacatgatgacagaatttaattttcttagcagcaag
ctcatggatcctggtgataaatgcagcatgactttactgaaaaggctttgtgatcttgaagagtggattgacttcactgtcggcagcacatgca
atctcacttgtttggtgtaatgaaagaagagaatgagaggtggaagggggatggtaatgttgaaaaaaagaatggtacagaggaaactgag
gttggagagagatggggtagatggtaagagatggagaaagagggaaggaaatggagagaaagacagagagacagagagagacacac
agagagacacacagagacagagaggaagggaaagggaaagagaaaggaagaggaagagggggaggggaaggggaaggggaag
gggaagggagagggagaaatgtggacactagccagatttaagggagaaattagggggttgccagtctgtccacctctgatggtggcaact
cagcagaaagctgctgggctcagtctggctttgttgagcaaccctgactccaccccttttcttccccacaaagcaagcttttaaagggaaggc
tttcttcattgaatgactgccacagtacg
SEQ ID NO: 39-TMEM119 promoter
gggggtgagcgagggctgctgggaccattgcagggaacaatgataatctaggcttggttcctacccagagagcacgcactcatccttcatg
cactcccctgttccaaaccctcactggctccgtactgcctccgaccttccgagactttagcctggctcctgtcaacatctctgacccttactaca
tgatcctctctttggtccatgctccagcctaatctaattgcggtggcttgtgcgtggtggcattcccagccaccatacctttacccacgctggtcc
ttccatgcggaatgcctttccagggcctgctttgcccgcttctgctcatacacaggcatgccctccaggatggcttcctacctctttcccttggg
ggattgatctctctgtcttggggttctcggagcccttgacctgacccctttctgtttggcaaaaaagtaatttacctcggtgtccttctccctggta
gtctgtgagctccccaaggctgggctgtgcctgattcacctctggaacttgcttagcacagtgcgtggcctgctgcaggtgttcattgagcact
tgccgaatgaatgcatgaatgaatgaatgaatgaatgaatgcaaggggctgctaatccacaggactcctcaggtcagccagacgtcccggt
tccaaggcctgccactgactcacctcaggaccctgcttgaaccattagaactcaccctgcctcactttccccctctgtgaaatggggctccaa
ctcctattcaagctactatcatttgggggcattgtgaggccacagatcccagaacatcagagtcagaggtagcccagaaagcttcccaccca
tccctacaaatgggaaactgaggtctggagagggaagggcagagttgggctccctgtctcaggctcggacccaccatcaggcctgtctcta
aaacgaatcccagctcccacgctgcaccctgagcctggaagcctgagccacacaaggacggggaattttccttcccacttccagaggcct
ctgaacctccctgagcttgtcccctttggagggtattgggcagcagcgtgggcagaaccccagctcactgtctgggggagcgctgcagga
cagccttgtctgtctgtctcagcctgccctggggacccgaggtcagggaggaagtgccgcatctggtcttccccagagcgagagtgtgagc
aagggtgggattgcgtgtggcccgagagtagcccctcccctccccctgtccccaccccaaaccctcttaatgaaatcaagctggccctgcg
gcccagccggggagggaggaaggaggagggacgggaggagggacgggaggagggagggcgggcaggcgccagcccagagca
gccccgggcaccagcacggactctctcttccagcccaggtgccccccactctcgctccattcggcgggagcacccagtcctgtacgccaa
ggaactggtgagtcctggggtcccctcct
SEQ ID NO: 40-MEF2 promoter
acatgcaggcactgtggccccgaaaaactcttcagaggtgagcggctttcaggatgagagggccaacaatggctcatctcctaaagacctc
gataaacataatcagtagccccagaaaacacaacagctgtcccaagcctatcccctttgcacctatctcagaaaaggcaacatgcagagtg
gtcaggagttcaagtcctcagggccctactctgaagctgtgaccctgacaggtcactttagcgctcagcttccctttcctcatctgtaaaatgg
gaatagaaactgaatctgcttcatggggccttgtaagtattacataagctgtcatatgcaaaatgtccatcacatagttcaccgccccaggcat
cctagaaactattaagtcacgataaggaggttgctactgtcctgggacctggggagctcggctgtccagtccgaaagggccttctcgccctc
gagaccccttcaagtccctttctctccgggcccgggtcccagagtccccttgtgcttgctcctgagggtgacctgcccgtgccccggggcg
agcacaggtgcggccctggccacaatgcgggcgtggcgagccgacacccggggaggccaagggcgcctcgagggagggagggag
gaggctgacccgggcgtcccgaaggacccgcccggccccggactccgggcggcagccggcctcgcgccccgcccccgccccggga
gccccgcccagtcccgtcgcgccgcgccgccccaccgcccgggttggctgccctggaggccacgcgcggcgatttgccacgcgccgc
gtcaccgggccgctcccggcctgggctcccgggggctggtcagggaggtggcggcggctgagcggcggagcgggggcggcagggc
gcgacgccgccgggccgccccgccctgggaggcgcccgggccctcatcaagtgaccagtatcccttccaggggaacacggtccttcag
aggaaagcgagctccaacccgcggccccggcgccaagccgccgtcatcttcttcctgtgccaggtgatcgtctcctcacccacccggaaa
aacatagtccgcccccacgtccttgtggaatagcgcccgcttccaagcaccgtgactctctttgccctacccttgtctttcccattccaattactc
tagaacccaccgagaggataattcagtcctgaaaagaaactgatggggagaagcgaggaagggaacccaggagggaggggaggcag
ggctgcggagggacaccgaggcggcggaggtagtgcgcacgcgcagcacagaacgagttccggtctggccgaggcttgtctcctaaaa
atagccccggtgtggggatccgtgcgcggatgtcccggcgagtcccgggctgaaagaggcggctccgggcggcgcgaagcgctggtg
gcgggcccgggctgcggcgtgtgcgcgcccgccagctgctccggagatacggtgagggccgcgggcagcggggctcagtccgcgac
SEQ ID NO: 41-oxP2 promoter
agtgcagcctcaggggttgaaggtgaaatgtgaaatttgctgtaattacttacccaaaatgtctgtattaaaggctattagaagcagttattttaa
gtacttctttatacatctttccctcttctgtttagtatttaaatgtgcaacaaaagtagtggcagatgttactcatctgaagaaactaaaacaagata
agtcagtggcaggagtggtggccggttaaagatctcttcgaagtgctttgttcagtaactgttgttttgtctcacattaagttgtaggtggggtgc
tgttaaagcttgtccactcacgatggtgttcagctgagacctgtgagttcaagttgtcttgcccagagttaaaggccataaaactaagacagtgt
cgttcctgtcttgggtgtaactcctaaaaaactagtcagttcttgcaataggaggaaaattattgcaaataaacctttgatttaccagcttcagtaa
gttgttcattctgtctctgtagctgtagtttttaccttaacagtttgaggctaccatttctcttcctttcatgcttatggggtccacacaaaccctgcta
ggctacctacagtataaaatcttaaaactccatgtggtgttgtgagctggtagcagatggactttcttttgcatctgctgtgtacagacttggcttt
gtactcttttcctgggggagagaaagtctttcttctctgctagttgattcctgtttttgtgaaacagctaagaaaacatctgtggcaggcagaaaa
attgacaaaggattgacaaacaccattagctgaaaattttccatttcattgcaactatgcattgtttactttactgtaaatatcttaatacatcatcctc
tgaatatgctggcagagaagctggagaactgtgatttcaattaaggttagtaattgatggtatctagtgttcaaaggctgaagcttgattaacat
ctgcttggacaaattgtcattgtgaagtggttttgatgtgcattgaagattatttctttctggcttatctgatgttgtggctgtgaaatgcttgtcttgg
gtttgcttatttttgtaaactacttcctttggctgtaaattgcagagcactggagctttaccaaaagtgcagtgtataatggtaagcttgtcctaata
agggaagcaagaagtgtatttatcacagacatgaaagctaaccgaggacttgagagactcaaactggtgcttttgtctctctctctctgtctttct
ctctctcacacacacactcacacactcacacacatgcacacacacacatacacacacacaaaaatgaagcacttactttagaaagattatggt
aagcatgctggctcagtcttgaacctttgtcacccctcacgttgcacaccaaagacataccctagtgattaaatgctgattttgtgtacgattgtc
cacggacgccaaaacaatcacagagctgcttgatttgttttaattaccagcacaaaatgccatcagtctgggacgtgatcgggcagaggtgt
SEQ ID NO: 42-hDNaseI(hyperactive)correct leader-WPRE.bGH
atgaggggcatgaagctgctgggggcgctgctggcactggcggccctactgcagggggccgtgtccctgaagatcgcagccttcaacat
caggacatttgggaggaccaagatgtccaatgccaccctcgtcagctacattgtgcagatcctgagccgctatgacatcgccctggtccag
gaggtcagagacagccacctgactgccgtggggaagctgctggacaacctcaatcaggatgcaccagacacctatcactacgtggtcagt
gagccactgggacggaagagctataaggagcgctacctgttcgtgtacaggcctgaccaggtgtctgcggtggacagctactactacgat
gatggctgcgagccctgcgggaacgacaccttcaaccgagagccattcattgtcaggttcttctcccggttcacagaggtcagggagtttgc
cattgttcccctgcatgcggccccgggggacgcagtagccgagatcgacgctctctatgacgtctacctggatgtccaagagaaatggggc
ttggaggacgtcatgttgatgggcgacttcaatgcgggctgcagctatgtgagaccctcccagtggtcatccatccgcctgtggacaagccc
caccttccagtggctgatccccgacagcgctgacaccacagctacacccacgcactgtgcctatgacaggatcgtggttgcagggatgctg
ctccgaggcgccgttgttcccgactcggctcttccctttaacttccaggctgcctatggcctgagtgaccaactggcccaagccatcagtgac
cactatccagtggaggtgatgctgaagtgaagtggcggccgctcgagctagcggccgctctagaagataatcaacctctggattacaaaatt
tgtgaaagattgactggtattcttaactatgttgctccttttacgctatgtggatacgctgctttaatgcctttgtatcatgctattgcttcccgtatgg
ctttcattttctcctccttgtataaatcctggttgctgtctctttatgaggagttgtggcccgttgtcaggcaacgtggcgtggtgtgcactgtgtttg
ctgacgcaacccccactggttggggcattgccaccacctgtcagctcctttccgggactttcgctttccccctccctattgccacggcggaac
tcatcgccgcctgccttgcccgctgctggacaggggctcggctgttgggcactgacaattccgtggtgttgtcggggaaatcatcgtcctttc
cttggctgctcgcctgtgttgccacctggattctgcgcgggacgtccttctgctacgtcccttcggccctcaatccagcggaccttccttcccg
cggcctgctgccggctctgcggcctcttccgcgtcttcgccttcgccctcagacgagtcggatctccctttgggccgcctccccgcatcgga
ctagaagcttgcctcgagcagcgctgctcgagagatctacgggtggcatccctgtgacccctccccagtgcctctcctggccctggaagttg
ccactccagtgcccaccagccttgtcctaataaaattaagttgcatcattttgtctgactaggtgtccttctataatattatggggtggagggggg
tggtatggagcaaggggcaagttgggaagacaacctgtagggcctgcggggtctattgggaaccaagctggagtgcagtggcacaatctt
ggctcactgcaatctccgcctcctgggttcaagcgattctcctgcctcagcctcccgagttgttgggattccaggcatgcatgaccaggctca
gctaatttttgtttttttggtagagacggggtttcaccatattggccaggctggtctccaactcctaatctcaggtgatctacccaccttggcctcc
caaattgctgggattacaggcgtgaaccactgctcccttccctgtccttctgattttgtaggtaaccacgtgcggaccga
SEQ ID NO: 43-hDNaseI(wild type)WPRE.bGH
atgaggggcatgaagctgctgggggcgctgctggcactggcggccctactgcagggggccgtgtccctgaagatcgcagccttcaacat
ccagacatttggggagaccaagatgtccaatgccaccctcgtcagctacattgtgcagatcctgagccgctatgacatcgccctggtccagg
aggtcagagacagccacctgactgccgtggggaagctgctggacaacctcaatcaggatgcaccagacacctatcactacgtggtcagtg
agccactgggacggaacagctataaggagcgctacctgttcgtgtacaggcctgaccaggtgtctgcggtggacagctactactacgatga
tggctgcgagccctgcgggaacgacaccttcaaccgagagccagccattgtcaggttcttctcccggttcacagaggtcagggagtttgcc
attgttcccctgcatgcggccccgggggacgcagtagccgagatcgacgctctctatgacgtctacctggatgtccaagagaaatggggct
tggaggacgtcatgttgatgggcgacttcaatgcgggctgcagctatgtgagaccctcccagtggtcatccatccgcctgtggacaagccc
caccttccagtggctgatccccgacagcgctgacaccacagctacacccacgcactgtgcctatgacaggatcgtggttgcagggatgctg
ctccgaggcgccgttgttcccgactcggctcttccctttaacttccaggctgcctatggcctgagtgaccaactggcccaagccatcagtgac
cactatccagtggaggtgatgctgaagtgaagtggcggccgctcgagctagcggccgctctagaagataatcaacctctggattacaaaatt
tgtgaaagattgactggtattcttaactatgttgctccttttacgctatgtggatacgctgctttaatgcctttgtatcatgctattgcttcccgtatgg
ctttcattttctcctccttgtataaatcctggttgctgtctctttatgaggagttgtggcccgttgtcaggcaacgtggcgtggtgtgcactgtgtttg
ctgacgcaacccccactggttggggcattgccaccacctgtcagctcctttccgggactttcgctttccccctccctattgccacggcggaac
tcatcgccgcctgccttgcccgctgctggacaggggctcggctgttgggcactgacaattccgtggtgttgtcggggaaatcatcgtcctttc
cttggctgctcgcctgtgttgccacctggattctgcgcgggacgtccttctgctacgtcccttcggccctcaatccagcggaccttccttcccg
cggcctgctgccggctctgcggcctcttccgcgtcttcgccttcgccctcagacgagtcggatctccctttgggccgcctccccgcatcgga
ctagaagcttgcctcgagcagcgctgctcgagagatctacgggtggcatccctgtgacccctccccagtgcctctcctggccctggaagttg
ccactccagtgcccaccagccttgtcctaataaaattaagttgcatcattttgtctgactaggtgtccttctataatattatggggtggagggggg
tggtatggagcaaggggcaagttgggaagacaacctgtagggcctgcggggtctattgggaaccaagctggagtgcagtggcacaatctt
ggctcactgcaatctccgcctcctgggttcaagcgattctcctgcctcagcctcccgagttgttgggattccaggcatgcatgaccaggctca
gctaatttttgtttttttggtagagacggggtttcaccatattggccaggctggtctccaactcctaatctcaggtgatctacccaccttggcctcc
caaattgctgggattacaggcgtgaaccactgctcccttccctgtccttctgattttgtaggtaaccacgtgcggaccga
SEQ ID NO: 44-human DNaseI mutant
atgaggggcatgaagctgctgggggcgctgctggcactggcggccctactgcagggggccgtgtccctgaagatcgcagccttcaacat
caggacatttgggaggaccaagatgtccaatgccaccctcgtcagctacattgtgcagatcctgagccgctatgacatcgccctggtccag
gaggtcagagacagccacctgactgccgtggggaagctgctggacaacctcaatcaggatgcaccagacacctatcactacgtggtcagt
gagccactgggacggaagagctataaggagcgctacctgttcgtgtacaggcctgaccaggtgtctgcggtggacagctactactacgat
gatggctgcgagccctgcgggaacgacaccttcaaccgagagccattcattgtcaggttcttctcccggttcacagaggtcagggagtttgc
cattgttcccctgcatgcggccccgggggacgcagtagccgagatcgacgctctctatgacgtctacctggatgtccaagagaaatggggc
ttggaggacgtcatgttgatgggcgacttcaatgcgggctgcagctatgtgagaccctcccagtggtcatccatccgcctgtggacaagccc
caccttccagtggctgatccccgacagcgctgacaccacagctacacccacgcactgtgcctatgacaggatcgtggttgcagggatgctg
ctccgaggcgccgttgttcccgactcggctcttccctttaacttccaggctgcctatggcctgagtgaccaactggcccaagccatcagtgac
cactatccagtggaggtgatgctgaagtga
SEQ ID NO: 45-AAV5 capsid sequence (VP1)
MSFVDHPPDWLEEVGEGLREFLGLEAGPPKPKPNQQHQDQARGLVLPGYNYLGPGNGL
DRGEPVNRADEVAREHDISYNEQLEAGDNPYLKYNHADAEFQEKLADDTSFGGNLGKA
VFQAKKRVLEPFGLVEEGAKTAPTGKRIDDHFPKRKKARTEEDSKPSTSSDAEAGPSGS
QQLQIPAQPASSLGADTMSAGGGGPLGDNNQGADGVGNASGDWHCDSTWMGDRVVT
KSTRTWVLPSYNNHQYREIKSGSVDGSNANAYFGYSTPWGYFDFNRFHSHWSPRDWQ
RLINNYWGFRPRSLRVKIFNIQVKEVTVQDSTTTIANNLTSTVQVFTDDDYQLPYVVGN
GTEGCLPAFPPQVFTLPQYGYATLNRDNTENPTERSSFFCLEYFPSKMLRTGNNFEFTYN
FEEVPFHSSFAPSQNLFKLANPLVDQYLYRFVSTNNTGGVQFNKNLAGRYANTYKNWF
PGPMGRTQGWNLGSGVNRASVSAFATTNRMELEGASYQVPPQPNGMTNNLQGSNTYA
LENTMIENSQPANPGTTATYLEGNMLITSESETQPVNRVAYNVGGQMATNNQSSTTAPA
TGTYNLQEIVPGSVWMERDVYLQGPIWAKIPETGAHFHPSPAMGGFGLKHPPPMMLIK
NTPVPGNITSFSDVPVSSFITQYSTGQVTVEMEWELKKENSKRWNPEIQYTNNYNDPQF
VDFAPDSTGEYRTTRPIGTRYLTRPL
SEQ ID NO: 46-AAV9 capsid sequence (VP1)
MAADGYLPDWLEDNLSEGIREWWALKPGAPQPKANQQHQDNARGLVLPGYKYLGPG
NGLDKGEPVNAADAAALEHDKAYDQQLKAGDNPYLKYNHADAEFQERLKEDTSFGG
NLGRAVFQAKKRLLEPLGLVEEAAKTAPGKKRPVEQSPQEPDSSAGIGKSGAQPAKKRL
NFGQTGDTESVPDPQPIGEPPAAPSGVGSLTMASGGGAPVADNNEGADGVGSSSGNWH
CDSQWLGDRVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANNLTSTVQVFT
DSDYQLPYVLGSAHEGCLPPFPADVFMIPQYGYLTLNDGSQAVGRSSFYCLEYFPSQML
RTGNNFQFSYEFENVPFHSSYAHSQSLDRLMNPLIDQYLYYLSKTINGSGQNQQTLKFSV
AGPSNMAVQGRNYIPGPSYRQQRVSTTVTQNNNSEFAWPGASSWALNGRNSLMNPGP
AMASHKEGEDRFFPLSGSLIFGKQGTGRDNVDADKVMITNEEEIKTTNPVATESYGQVA
TNHQSAQAQAQTGWVQNQGILPGMVWQDRDVYLQGPIWAKIPHTDGNFHPSPLMGGF
GMKHPPPQILIKNTPVPADPPTAFNKDKLNSFITQYSTGQVSVEIEWELQKENSKRWNPE
IQYTSNYYKSNNVEFAVNTEGVYSEPRPIGTRYLTRNL

### Definitions

The term “an enzyme which has a deoxyribonuclease (DNase) activity” is used herein to refer to an enzyme capable of hydrolytic cleavage of phosphodiester linkages in the DNA backbone.

As used herein, the terms “deoxyribonuclease” and “DNase” are used to refer to any enzyme that catalyzes the hydrolytic cleavage of phosphodiester linkages in the DNA backbone. A wide variety of deoxyribonucleases is known and can be used in the methods of the present invention. Non-limiting examples of DNases useful in the methods of the present invention include, e.g., DNase I (e.g., recombinant human DNase I (rhDNase I) or bovine pancreatic DNase I), analogues of DNase I (such as, e.g., DNase X, DNase gamma, and DNAS1L2), DNase II (e.g., DNase II-alpha, DNase II-beta), phosphodiesterase I, lactoferrin, and acetylcholinesterase. Also encompassed by the present invention are DNase enzymes which have an extended half-life (e.g., albumin and/or Fc fusions, or protected from binding to actin by modification of actin binding-site; see, e.g., Gibson et al., (1992) J. Immunol. Methods, 155, 249-256). The actin binding site of DNase I can be mutated, for example, at the following residues: Gln-9, Glu-13, Thr-14, His-44, Asp-53, Tyr-65, Val-66, Val-67, Glu-69, Asn-74, Ala-114 of recombinant human DNase I (SEQ ID NO: 4). For example, in human DNase I hyperactive variant comprising the sequence of SEQ ID NO: 5, the Ala-114 residue is mutated. Exemplary mutations include H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P70T, S94N:Y96S, S94N:Y96T (in the sequence of SEQ ID NO: 4). Also encompassed are mutations in DNase I with increased DNase I activity. Non-limiting examples of such mutations are, e.g., Q9R, E13R, E13K, T14R, T14K, H44R, H44K, N74K, and A114F of recombinant human DNase I (SEQ ID NO: 4). For example, a combination of the Q9R, E13R, N74K and A114F mutations is found in the hyperactive DNase I comprising the sequence of SEQ ID NO: 5. DNase I cleaves DNA preferentially at phosphodiester linkages adjacent to a pyrimidine nucleotide, yielding 5′-phosphate-terminated polynucleotides with a free hydroxyl group on position 3′, on average producing tetranucleotides. DNase I acts on single-stranded DNA, double-stranded DNA, and chromatin.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, up to ±10%, up to ±5%, and up to ±1% of a given value. Alternatively, particularly with respect to biological systems or processes, the term can mean within an order of magnitude, such as within 2-fold, of a value. Where particular values are described in the application and claims, unless otherwise stated, the term “about” is implicit and in this context means within an acceptable error range for the particular value.

The term “protein misfolding” is used herein to refer to the formation of β-structures and/or aggregates and/or prions of proteins (e.g., human proteins such as Tau, β-amyloid, SOD1, TDP-43,α-synuclein, poly-Q, huntigtin, transthyretin, p53, etc.).

The term “microbial DNA” is used to refer to DNA from bacteria, fungi, and intracellular and extracellular parasites. Non-limiting examples of the source organisms include, e.g., P. gingivalis, E. coli, B. burgdorferi, C. albicans, Tetzerella alzheimeri, Tetzosporium hominis, Treponema spp., and Propionibacterium spp.

The term “human DNA” is used to refer to DNA from human, including but not limited to, DNA secreted by cells from microglia or neutrophil extracellular traps (NETs).

The terms “extracellular DNA”, “cell-free DNA” and “cfDNA” are used interchangeably to refer to extracellular DNA (e.g., of eukaryotic, viral, archaeal, prokaryotic, intracellular and extracellular parasites origin), including DNA in extracellular vesicles (e.g., exosomes and microvesicles), found in any bodily fluid and tissue. The cfDNA relevant for the present invention can be found in CSF or nervous tissues and encompasses DNA from the extracellular vesicles (e.g., exosomes and microvesicles) in the CSF.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The terms “individual”, “subject”, “animal”, “patient”, and “mammal” are used interchangeably to refer to mammals, including humans, veterinary animals (e.g., cats, dogs, cows, horses, sheep, pigs, etc.) and experimental animal models of diseases (e.g., mice, rats).

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a combination of DNase and another compound) the effective amount of the combination may or may not include amounts of each ingredient that would have been effective if administered individually.

As used herein, the term “promoter” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “tissue specific” promoter may be preferentially active in specific types of tissues or cells.

The term “nervous system-specific expression” as used herein refers to a predominant or exclusive expression in the nervous system, i.e., expression to a substantially greater extent than in other tissues and organs.

The term “nervous system-specific promoter” is used herein to refer to a promoter which is predominantly or exclusively active in a nervous system cell and directs/initiates transcription in the nervous system (e.g., central nervous system (CNS), including brain, and/or enteric nervous system (ENS)) to a substantially greater extent than in other tissues and organs. In this context, the term “predominantly” means that at least 50% of said promoter-driven expression, more typically at least 90% of said promoter-driven expression (such as 100% of said promoter expression) occurs in cells of the nervous system. The ratio of nervous system expression to non-nervous system expression can vary between different nervous system-specific promoters. In some embodiments, a nervous system-specific promoter may preferentially direct/initiate transcription in a particular CNS and/or ENS cell type (e.g., neurons, glial cells [e.g., oligodendrocytes, astrocytes, ependymal cells, microglia, Schwann cells, satellite cells], enteric neurons, intrinsic primary afferent neurons, interneurons, motor neurons, etc.). Some nervous system-specific promoters useful in the expression cassettes of the invention include at least one, typically several, neuron nuclear factor binding sites. nervous system-specific promoters useful in the expression cassettes of the invention can be constitutive or inducible promoters.

Some non-limiting examples of nervous system promoters useful in the expression cassettes of the invention include microglia-specific promoters (e.g., F4/80, CD68, TMEM119, CX3CR1, CMV, and Iba1 promoters), myeloid-specific promoters (e.g., TTR, CD11b, and c-fes promoters), neuron-specific promoters (e.g., CMV, NSE, synapsin [SynI, SynII], CamKII , CaMKII, GfaABC1D-dYFP, and VGLUT1 promoters), and other neural and glial cell (e.g., oligodendrocytes, astrocytes) type-specific promoters (e.g., glial fibrillary acidic protein [GFAP] promoter).

In some embodiments, e.g., when nervous system targeting is mediated by a capsid protein, a nucleic acid encoding an enzyme which has a DNase activity can be operably linked to a promoter that allows for efficient systemic expression (e.g., EF1a promoter).

The term “diseases associated with protein misfolding” is used herein to refer to neurodegenerative, neurodevelopmental, autoimmune, oncological, or other diseases associated with protein misfolding. In some embodiments, such diseases are associated with formation of neurotoxic aggregates. Non-limiting examples of diseases associated with protein misfolding include Alzheimer's disease, Parkinson's disease, Amyotrophic Lateral Sclerosis (ALS), multiple sclerosis, CADASIL Syndrome, stroke, Friedreich's ataxia, cerebellar ataxia, spinocerebellar ataxia, dementia (e.g., fronto-temporal dementia, frontotemporal dementia with parkinsonism-17 (FTDP-17), familial Danish dementia, and familial British dementia), prion-caused diseases, Lewy body diseases, amyloidosis (e.g., hereditary cerebral haemorrhage with amyloidosis, senile systemic amyloidosis), spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm; dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia and related disorders), Shy-Drager syndrome, spinal muscular atrophy, primary lateral sclerosis, hereditary spastic paraplegia, peroneal muscular atrophy (Charcot-Marie-Tooth), hypertrophic interstitial polyneuropathy (Dejerine-Sottas), other neurodegenerative diseases, nervous system tumors (e.g., astrocytoma, gliomas, glioblastoma, ependymoma, medulloblastoma, CNS lymphoma, craniopharyngioma, glioblastoma, meningioma, pituitary carcinomas, pituitary adenoma, neurofibromatosis, embryonal tumors, tumors of the pineal region, tumors of meninges, choroid plexus tumors, neuronal and mixed neuronal-glial tumors, germ cell tumors), secondary neurodegeneration (e.g., resulting from destruction of neurons by neoplasm, edema, hemorrhage, stroke, trauma, immune attack, hypoxia, poisoning, metabolic defects, or infections).

The phrase “pharmaceutically acceptable”, as used in connection with compositions of the invention, refers to molecular entities and other ingredients of such compositions that are physiologically tolerable and do not typically produce untoward reactions when administered to a subject (e.g., a mammal such as a human). As used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “cytostatic and/or cytotoxic chemotherapy” and “chemotherapy” are used interchangeably herein to refer to a therapy involving administering of a cytostatic and/or cytotoxic agent.

The terms “anti-cancer agent” and “anti-cancer chemotherapeutic agent” are used herein to refer to any chemical compound, which is used to treat cancer. Anti-cancer chemotherapeutic agents are well known in the art (see, e.g., Gilman A. G., et al., The Pharmacological Basis of Therapeutics, 8th Ed., Sec 12:1202-1263 (1990)). Specific examples of chemotherapeutic agents are provided throughout the specification.

As used herein, the terms “radiotherapy”, “radiation therapy”, and “RT” are used interchangeably to refer to the medical use of ionizing radiation as part of a cancer treatment to damage the DNA of malignant cells, either directly or by creating charged particles within the affected cells that damage the DNA. Commonly used types of radiation therapy encompassed by the present invention include, e.g., external beam radiation therapy (EBRT or XRT), brachytherapy/sealed source radiation therapy, and systemic radioisotope therapy/unsealed source radiotherapy

The terms “side effect of a radiotherapy” or “side effect of a radiation therapy” as used herein refer to an undesirable and unintended, although not necessarily unexpected, result of a radiation therapy. Which side effects develop depend on the area of the body being treated, the dose given per day, the total dose given, the patient's general medical condition, and other treatments given at the same time, and may include, e.g., body weight loss, headaches, fatigue, nausea, vomiting, troubles with memory and speech, dementia, and a second cancer.

The term “catabolic state” as used herein refers to a condition characterized by a rapid weight loss and loss of fat and skeletal muscle mass, which may occur in a background of chemotherapy or chemoradiation therapy. Associated clinical events include, for example, immunosuppression, muscle weakness, predisposition to pulmonary embolism, thrombophlebitis, and altered stress response.

The terms “drugs for the treatment of neurodegenerative diseases” interchangeably herein to refer to a therapy used for the treatment of neurodegenerative and neurodevelopmental diseases including the treatment of cognitive function, loss of memory, inability to learn, decline in behavior and function, treatment of protein misfolding, antiamyloid, neurotransmitter based, neuroprotectors drugs.

As used herein, the terms “viral vector” and “viral construct” refer to a recombinant viral construct that comprises one or more heterologous nucleotide sequences (e.g., a nucleotide sequence encoding an enzyme which has a DNase activity). In some embodiments, the viral vector is replication deficient. In some embodiments, viral structural and non-structural coding sequences are not present in the viral vector and are provided during viral vector production in trans by a vector, such as a plasmid, or by stably integrating the sequences into a packaging cell line. Depending on the virus, a viral vector can be packaged within a capsid (e.g., an AAV vector) and/or a lipid envelope (e.g., a lentiviral vector).

The term “polyadenylation signal”, as used herein, relates to a nucleic acid sequence that mediates the attachment of a polyadenine stretch to the 3′ terminus of the mRNA. Non-limiting examples of polyadenylation signals which can be used on the expression constructs of the invention include, e.g., the SV40 early polyadenylation signal, the SV40 late polyadenylation signal, the HSV thymidine kinase polyadenylation signal, the protamine gene polyadenylation signal, the adenovirus 5 EIb polyadenylation signal, the bovine growth hormone polyadenylation signal, the human variant growth hormone polyadenylation signal and the like.

As used in this specification and the appended claims, the singular forms “a,” “an,” and “the” include plural references unless the context clearly dictates otherwise.

In accordance with the present invention there may be employed conventional pharmacology and molecular biology techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985)); Transcription and Translation (B. D. Hames & S. J. Higgins, eds. (1984)); Animal Cell Culture (R. I. Freshney, ed. (1986); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994); among others.

### DNase Enzyme Targeting to Nervous Dystem

The inventors have unexpectedly found that targeting expression of an enzyme which has a DNase activity to the nervous system can bring about an enhanced clearance of disease-associated cfDNA from the CSF circulation and nervous system tissues. As demonstrated in the Examples section, below, cfDNA can lead to the protein misfolding. Degradation of cfDNA via localized expression of an enzyme which has DNase activity can have a therapeutic and preventive effect.

As discussed throughout the application, an enzyme which has DNase activity can be efficiently expressed in nervous system cells, e.g., through the use of viral vectors (e.g., adeno-associated viral (AAV) vectors, retroviral vectors [e.g., lentiviral vectors], or adenoviral vectors), liposomes, nanoparticles carrying a DNase transgene, or naked DNA.

An adult human brain ventricles are filled with CSF which bathes and cushions the brain and spinal cord within their bony confines. The BBB and the blood-cerebrospinal fluid barrier (BCSFB) prevents penetration of microorganisms and viruses and their DNA to the CSF or brain. However, the altered function of BBB or BCSFB lead to the increased circulation of the nucleic acids in CSF. Another way for microorganisms and viruses to gain an access to the CNS and brain is via their spread through cranial nerves and propagation along the olfactory and trigeminal tracts. See, e.g., Riviere et al., (2002) Oral Microbiology and Immunology, 17(2):113-118; Olsen I., & Singhrao, S. K. (2015) Journal of Oral Microbiology, 7(1):29143; Ramya, V., & Bhuvaneshwarri, P. P. N. M. Biosci Biotech Res Asia. 2014, 11(1):259-61.

The present inventors have found that transfection of nervous system cells to express an enzyme which has DNase activity provides a reduction in cfDNA in CSF and nervous tissues, along with substantial and surprising improvement in diseases and conditions associated with protein misfolding.

In some embodiments, expression of DNase enzyme in the nervous system is accomplished by use of nucleic acid expression cassettes that are predominantly expressed in the cells of nervous system. In some embodiments, such expression cassettes can comprise one or more of the following elements: (a) a nervous system locus control element; (b) a nervous system-specific promoter located 3′ to the nervous system locus control element; (c) a coding sequence located 3′ to the nervous system-specific promoter, said coding sequence encoding a polypeptide, e.g. a deoxyribonuclease; (d) a polyadenylation signal located 3′ to the coding sequence; and (e) an intron located 3′ to the nervous system-specific promoter and 5′ to the polyadenylation signal. The elements (a), (b), (c), (d) and (e) can be operably linked to express the polypeptide encoded by the coding sequence. In some embodiments, the expression cassettes of the invention direct expression of a therapeutic amount of a polypeptide in CSF and nervous system cells for a period of at least 100 days (such as at least 200 days, at least 300 days, at least 400 days, or at least 500 days, or at least 5000 days, or at least 30,000 days). In some embodiments, the polypeptide is a DNase. The DNase may be any DNase described herein, e.g., DNase I.

In another aspect, the present invention provides methods for treating a disease or condition associated with protein misfolding due to microbial and/or viral cfDNA seeding activity in CSF or locally increased levels of cfDNA in nervous system tissues and fluids (e.g., without general elevation of cfDNA level in non-C SF bodily fluids). Non-limiting examples of diseases and conditions treatable by the methods of the invention include, e.g., any disease or condition described herein or in any of U.S. Pat. Nos. 7,612,032; 8,388,951; 8,431,123; 8,535,663; 8,710,012; 8,796,004; 8,871,200; 8,916,151; 9,072,733; 9,248,166; 9,770,492; U.S. Pat. Appl. Pub. Nos. US20170056482, US20170100463, US20150110769, and Int. Appl. Pub. No. WO2016/190780, all of which are incorporated by reference herein in their entireties. In one embodiment, the method comprises the steps of: (1) introducing into the nervous system of a subject a vector comprising a nucleic acid expression cassette, said expression cassette comprising: (a) a nervous system-specific promoter; (b) a coding sequence located 3′ to the nervous system-specific promoter, said coding sequence encoding an enzyme which has a DNase activity (e.g., any DNAs described herein); (c) a polyadenylation signal located 3′ to the coding sequence; and optionally (d) an intron located 3′ to the nervous system-specific promoter and 5′ to the polyadenylation signal. Elements (a), (b), (c), and (d) are operably linked to express the polypeptide encoded by the coding sequence; and (2) expressing a therapeutic amount of said polypeptide in the nervous system. In some embodiments of the methods of this aspect of the invention, a therapeutic amount of the polypeptide is expressed for at least 100 days (such as at least 200 days, at least 300 days, at least 400 days, at least 500 days, at least 5000 days and at least 30,000 days).

The nucleic acid expression vectors and cassettes of this aspect of the invention are predominantly expressed in the nervous system, e.g., at least 50% of the expression of the encoded polypeptide occurs within the nervous system. More typically, at least 90% of the expression occurs within the nervous system. Some of the expression vectors and cassettes of this aspect of the invention are exclusively expressed within the nervous system.

Specific locus control elements may be incorporated into the expression cassettes of the invention that are capable of enhancing the expression of nucleic acid molecules in nervous system cells, and confer copy number dependent, position independent expression on the expression cassette. Some nervous system locus control regions useful in the expression cassettes of the invention include, e.g., a matrix attachment region and/or a nervous system-specific enhancer element.

In addition to a promoter, an expression cassette may contain one or more additional transcription initiation, termination, and/or enhancer sequences; RNA processing signals (such as , e.g., splicing and polyadenylation (poly A) signals); sequences that stabilize cytoplasmic mRNA; sequences that enhance translation efficiency (e.g., Kozak consensus sequence, such as 5′-GCCGCCACC-3′ [SEQ ID NO: 33]); sequences that enhance protein stability; and sequences that enhance secretion of the encoded polypeptide. Non-limiting examples of suitable polyA sequences include, e.g., SV40, SV50, bovine growth hormone (bGH), human growth hormone, and synthetic polyAs. Non-limiting examples of suitable enhancers include, e.g., nPE2 enhancer, Gal4 enhancer, foxP2 neuron-specific enhancer, Mef2 microglia-specific enhancer, alpha fetoprotein enhancer, TTR minimal promoter/enhancer, LSP (TH-binding globulin promoter/alpha 1-microglobulin/bikunin enhancer), and Serpinl enhancer. In one embodiment, the expression cassette comprises one or more expression enhancers. In one embodiment, the expression cassette contains two or more expression enhancers. These enhancers may be the same or may differ from one another. For example, an enhancer may include an Alpha mic/bik enhancer. This enhancer may be present in two copies which are located adjacent to one another. Alternatively, the dual copies of the enhancer may be separated by one or more sequences.

In still another embodiment, the expression cassette further contains an intron. Non-limiting examples of introns include, e.g., the Promega intron, a truncated chimeric intron (T-chimeric intron), and introns described in Int. Pat. Appl. Pub. No. WO 2011/126808.

Optionally, one or more sequences may be selected to stabilize mRNA. An example of such a sequence is a modified WPRE sequence, which may be engineered upstream of the polyA sequence and downstream of the coding sequence (see, e.g., Zanta-Boussif et al., Gene Therapy (2009) 16:605-619).

In yet another embodiment, the expression cassette and/or vector further contains a post-transcriptional regulatory element (PRE). An exemplary post-transcriptional regulatory element is the Woodchuck hepatitis virus post-transcriptional regulatory element (WPRE). For example, a sequence from the WPRE is shown in SEQ ID NO: 16 or is part of SEQ ID NO: 42 or 43. The WPRE may be effective to increase expression of the protein (e.g., DNase). The WPRE may be modified so as not to produce an active form of a truncated X protein that is encoded in the WPRE. One such modified WPRE is provided in at least the sequence of SEQ ID NO: 16.

In some embodiments, the cassettes of the invention direct the expression of a therapeutic amount of the polypeptide encoded by the coding sequence for an extended period, typically greater than 200 days, and in some instances greater than 30000 days. Expression of the polypeptide encoded by the coding sequence can be measured by any art-recognized means, such as, e.g., by antibody-based assays, such as a Western Blot or an ELISA assay. Again, by way of non-limiting example, expression of the polypeptide encoded by the coding sequence within the expression cassette can be measured in a bioassay that detects an enzymatic or biological activity of the polypeptide.

Upon entry into the nervous system cells (e.g., neurons, microglial cells), the vectors can remain episomal (i.e., do not integrate into the genome of a host cell), or can integrate into the host cell genome. Non-limiting examples of episomal vectors include adenoviral vectors, and examples of vector that integrate into the host cell genome include retroviral vectors.

In some embodiments of the invention, the expression vectors comprising a nucleotide sequence encoding an enzyme which has DNase activity, are delivered to cells of nervous system in the form of liposomes in which the DNA is associated with one or more lipids, such as DOTMA (1,2-diolcyloxypropyl-3-trimethyl ammonium bromide) and DOPE (dioleoylphosphatidylethanolamine). In some embodiments, cationic liposomes containing DOTMA are effective to target expression vectors of the invention to the nervous system. In some embodiments, lactoferrin and/or poly L-lysine and/or polyethylenemine and/or chitosan is conjugated to an expression vector, with the conjugate effectively targeted to the nervous system after direct introduction into a subject.

In some embodiments, the expression vector is packaged into a nanovector or is encapsulated by a nanovector.

In some embodiments, the expression vector is packaged into phosphoramidite nanoparticles.

Various devices have been developed for enhancing the availability of the nucleic acids of the invention encoding enzymes which has DNase activity to the target cells, including, e.g., catheters or implantable materials containing DNA (G. D. Chapman et al., Circulation Res. 71:27-33 (1992)) and needle-free, jet injection devices which project a column of liquid directly into the target tissue under high pressure (P. A. Furth et al., Anal Biochem. 20:365-368 (1992); H. L. Vahlsing et al., J. Immunol. Meth. 175:11-22 (1994); F. D. Ledley et al., Cell Biochem. 18A:226 (1994)).

Another approach to targeted nervous system delivery of the nucleic acids of the invention encoding enzymes which has DNase activity to the cells of nervous system is the use of molecular conjugates, which consist of protein or synthetic ligands to which a nucleic acid-binding agent has been attached for the specific targeting of nucleic acids to cells (R. J. Cristiano et al., Proc. Natl. Acad. Sci. USA 90:11548-52 (1993); B. A. Bunnell et al., Somat. Cell Mol. Genet. 18:559-69 (1992); M. Cotten et al., Proc. Natl. Acad. Sci. USA 89:6094-98 (1992)). This gene delivery system has been shown to be capable of targeted delivery to many cell types through the use of different ligands (R. J. Cristiano et al., Proc. Natl. Acad. Sci. USA 90:11548-52 (1993)).

Non-limiting examples of routes of administration for the expression vectors of the invention include, e.g., intravenous, intraarterial, intracerebral, intracerebroventricular (i.c.v.), intraparenchymal injections, intrastriatal, intraspinal, intrathecal, subarachnoid injection, enteral (e.g., oral), intramuscular, intraperitoneal, etc.

The expression vectors of the invention can be introduced directly into the CSF or nervous system tissues by any art-recognized means, such as by direct injection into the brain or ependymal canal.

In some embodiments, the delivery is performed as follows. The brain, cor, spine, ventricles are visualized through a ventral midline incision. A vector/liposome/naked DNA in 1 ml of various solutions containing heparin to prevent clotting is intraspinally using a needle over approximately 30 seconds.

The efficiency of nervous system delivery, expression and secretion of the enzyme which has DNase activity can be determined, e.g., by assaying the level of the enzyme in the CSF, using immunological assays (e.g., assays with antibodies recognizing the enzyme such as., e.g., a radioimmune assay (RIA) (HGH-TGES 100T kit from Nichols Institute, San Juan Capistrano, Calif., USA). In experimental animal models, the efficiency of nervous system delivery, expression and secretion of the enzyme which has DNase activity can be determined, e.g., by measuring DNase I activity in CSF or CNS homogenates.

### Parvovirus Vectors, Including Adeno-Associated Virus Vectors

In certain embodiments, the vector used in the invention is a parvovirus vector, such as an adeno-associated viral (AAV) vector. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B 19 virus, and any other autonomous parvovirus now known or later discovered. Other autonomous parvoviruses are known to those skilled in the art. See, e.g., Bernard N. Fields et al., Virology, Vol. 2, Chapter 69 (4th ed., Lippincott-Raven Publishers).

In some embodiments, the parvovirus vector is a single-stranded parvovirus vector, such as an AAV vector. AAV (genus Dependovirus) normally infects humans (e.g., serotypes 1, 2, 3A, 3B, 4, 5, and 6) or primates (e.g., serotypes 1 and 4), or other warm-blooded animals (e.g., bovine, canine, equine, and ovine AAVs). Further information on parvoviruses and other members of the Parvoviridae is provided, e.g., in Kenneth I. Berns, “Parvoviridae: The Viruses and Their Replication,” Chapter 69 in Fields Virology (3d Ed. 1996).

AAV vectors disclosed herein may be derived from any AAV serotype, including combinations of serotypes (e.g., “pseudotyped” AAV) or from various genomes (e.g., single-stranded or self-complementary). A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences). Non-limiting examples of AAV serotypes which can be used to develop the AAV expression vectors of the invention include, e.g., AAV serotype 1 (AAV1), AAV2, AAV3 (including types 3A and 3B), AAV4, AAV5, AAV6, AAV7, AAV8, AAV9, AAV10, AAV11, AAV12, AAVrh10 (as disclosed, e.g., in U.S. Pat. No. 9,790,472, Int. Pat. Appl. Pub. No. WO2017180857 and WO2017/180861), AAVLK03 (as disclosed, e.g., in Wang et al., Mol. Ther., 2015, 23(12):1877-1887), AAVhu37 (as disclosed, e.g., in Int. Pat. Appl. Pub. No. WO2017180857), AAVrh64R1 (as disclosed, e.g., in Int. Pat. Appl. Pub. No. WO2017180857), Anc80 (based on a predicted ancestor of serotypes AAV1, AAV2, AAV8 and AAV9; see Zinn et al., Cell Rep., 2015, 12(67): 1056-1068), avian AAV, bovine AAV, canine AAV, equine AAV, ovine AAV, and any other AAV now known or later discovered. See, e.g., Fields et al., Virology, volume 2, chapter 69 (4th ed., Lippincott-Raven Publishers).

Recently, a number of putative new AAV serotypes and clades have been identified (see, e.g., Gao et al., (2004) J. Virology 78:6381-6388; Moris et al., (2004) Virology 33-:375-383). The genomic sequences of the various serotypes of AAV and the autonomous parvoviruses, as well as the sequences of the terminal repeats, Rep proteins, and capsid subunits are known in the art, by way of example, Srivistava et al., (1983) J. Virology 45:555; Chiorini et al., (1998) J. Virology 71:6823; Chiorini et al, (1999) J. Virology 73:1309; Bantel-Schaal et al., (1999) J. Virology 73:939; Xiao et al., (1999) J. Virology 73:3994; Muramatsu et al., (1996) Virology 221: 208; Shade et al., (1986) J. Virol. 58:921; Gao et al., (2002) Proc. Nat. Acad. Sci. USA 99:11854; Moris et al., (2004) Virology 33-: 375-383; GenBank Accession number U89790; GenBank Accession number J01901; GenBank Accession number AF043303; GenBank Accession number AF085716; GenBank Accession number NC 006152; GenBank Accession number Y18065; GenBank Accession number NC 006260; GenBank Accession number NC 006261; International Patent Publication Nos. WO 00/28061, WO 99/61601, WO 98/11244; and U.S. Pat. No. 6,156,303, the disclosures of which are incorporated by reference herein for teaching parvovirus and AAV nucleic acid and amino acid sequences.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, VP2 and VP3) form the capsid. The terminal 145 nucleotides are self-complementary and are organized so that an energetically stable intramolecular duplex forming a T-shaped hairpin may be formed. These hairpin structures function as an origin for viral DNA replication, serving as primers for the cellular DNA polymerase complex. Following wtAAV infection in mammalian cells the Rep genes (i.e. Rep78 and Rep52) are expressed from the P5 promoter and the P19 promoter, respectively and both Rep proteins have a function in the replication of the viral genome.

In some embodiments, recombinant AAV (rAAV) vectors comprise one or more nucleotide sequences of interest that are flanked by at least one parvoviral or AAV inverted terminal repeat sequence (ITR). Such rAAV vectors can be replicated and packaged into viral particles when produced in a packaging cell that is expressing AAV rep and cap gene products (i.e. AAV Rep and Cap proteins). The terms “AAV Cap protein” or “AAV capsid protein”, as used herein, refer to a polypeptide having at least one functional activity of a native AAV Cap protein (e.g., VP1, VP2, VP3). Examples of functional activities of Cap proteins (e.g., VP1, VP2, VP3) include the ability to induce formation of a capsid, facilitate accumulation of single-stranded DNA, facilitate AAV DNA packaging into capsids (i.e., encapsidation), bind to cellular receptors, and facilitate entry of the virion into host cells.

In some embodiments, the AAV vectors may comprise desired proteins or protein variants. A “variant” as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both.

AAV vectors are packaged into AAV viral capsids. The sequence of an AAV viral capsid protein defines numerous features of a particular AAV vector. For example, the capsid protein affects capsid structure and assembly, interactions with AAV nonstructural proteins such as Rep and AAP proteins, interactions with host body fluids and extracellular matrix, clearance of the virus from the blood, vascular permeability, antigenicity, reactivity to neutralizing antibodies, tissue/organ/cell type tropism, efficiency of cell attachment and internalization, intracellular trafficking routes, virion uncoating rates, among others. The sequence of a capsid protein (e.g., VP3) may be altered to enhance delivery to the nervous system.

AAV constructs may further comprise a sequence encoding one or more capsid proteins (VP1 and/or VP2, and/or VP3 capsid proteins, preferably just VP3 capsid protein) which package the above-mentioned polynucleotide sequence. The sequences coding for the capsid protein(s) for use in the context of the present invention may be taken from any of the known 42 serotypes, such as, e.g., AAV1, AAV2, AAV3, AAV4, AAV5, AAV6, AAV7, AAV8, AAV9, Anc80, or newly developed AAV-like particles obtained by, e.g., capsid shuffling techniques and/or use of AAV capsid libraries. For some non-limiting examples of capsid protein sequences, see, e.g., U.S. Pat. Nos. 9,790,472; 9,677,089; 7,282,199; Int. Pat. Appl. Publ. Nos. WO 2015/054653, WO2017/180857 (AAV8, AAV9, AAVrh10, AAVhu37, AAVrh64R1), WO2017/180861 (AAVrh10), WO2017/180854 (AAV8 mutants), and Wang et al., Mol. Ther., 2015, 23(12):1877-1887 (AAV8, AAVrh10, AAV3B, and AAVLK03). When the sequences encoding the capsid proteins derive from a different AAV serotype as the ITRs, the AAV construct is known as a “hybrid” parvovirus genome (i.e., in which the AAV capsid and the AAV terminal repeat(s) are from different AAV) as described in Int. Pat. Appl. Publ. No. WO 00/28004 and Chao et al., (2000) Molecular Therapy 2:619.

In some embodiments, the AAV capsid protein(s) mediates efficient targeting of the AAV vector to the nervous system. In some embodiments, the invention encompasses the use of AAV capsid mutants which enhance nervous system targeting and/or nervous system specificity. Non-limiting examples of such nervous system-specific point mutations to the AAV8 capsid sequence include, e.g., S279A, S671A, K137R, and T252A, as well as AAV8 capsid mutations disclosed in Int. Pat. Appl. Pub. No. WO2017/180854 (e.g., AAV3G1, AAVT20 or AAVTR1, VP3 mutations in amino acids 263-267 [e.g., 263NGTSG267->SGTH (“NGTSG” disclosed as SEQ ID NO: 47 and “SGTH” disclosed as SEQ ID NO: 48) or 263NGTSG267->SDTH (“NGTSG” disclosed as SEQ ID NO: 47 and “SDTH” disclosed as SEQ ID NO: 49)] and/or amino acids 457-459 [e.g., 457TAN459->SRP], and/or amino acids 455-459 [e.g., 455GGTAN459 ->DGSGL (“GGTAN” disclosed as SEQ ID NO: 50 and “DGSGL” disclosed as SEQ ID NO: 51)] and/or amino acids 583-597).

The AAV vectors disclosed herein include a nucleic acid encoding an enzyme which has a DNase activity, such as, e.g., DNase I. In various embodiments, the nucleic acid also may include one or more regulatory sequences allowing expression and secretion of the encoded enzyme, such as e.g., a promoter, enhancer, polyadenylation signal, an internal ribosome entry site (IRES), a sequence encoding a protein transduction domain (PTD), a secretory signal sequence, and the like. Thus, in some embodiments, the nucleic acid may comprise a promoter region operably linked to the coding sequence to cause or improve expression of the protein of interest in transfected cells. Such a promoter may be ubiquitous, cell- or tissue-specific, strong, weak, regulated, chimeric, etc., for example to allow efficient and stable production of the protein in the nervous system. The promoter may be homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. Other promoters that may be used include promoters that are tissue specific, e.g., for nervous system. Non-limiting examples of nervous system-specific promoters include, e.g., microglia-specific promoters (e.g., F4/80, CD68, TMEM119, CX3CR1, CMV, and Iba1 promoters), myeloid-specific promoters (e.g., TTR, CD11b, and c-fes promoters), neuron-specific promoters (e.g., CMV, NSE, synapsin [SynI, SynII], CamKII , α-CaMKII, GfaABC1D-dYFP, and VGLUT1 promoters), and other neural and glial cell (e.g., oligodendrocytes, astrocytes) type-specific promoters (e.g., glial fibrillary acidic protein [GFAP] promoter).

The AAV constructs of the invention may also contain non-resolvable terminal repeats. The expression “non-resolvable terminal repeat”, as used herein, relates to terminal repeats which are not recognized by and resolved (i.e., “nicked”) by the AAV Rep proteins, such that resolution of the terminal repeat is substantially reduced (e.g., by at least about 50%, 60%, 70%, 80%. 90%, 95%, 98% or greater as compared with a resolvable terminal repeat) or eliminated. Such non-resolvable terminal repeats may be naturally-occurring terminal repeat sequences (including altered forms thereof) and, for example, can be derived from a parvovirus, including an AAV, or can be from another virus or, as a further alternative, can be partially or completely synthetic. The non-resolvable terminal repeat may be a non-AAV viral sequence that is not recognized by the AAV Rep proteins, or it can be an AAV terminal repeat that has been modified (e.g., by insertion, substitution and/or deletion) so that it is no longer recognized by the AAV Rep proteins. Further, a non-resolvable terminal repeat can be any terminal repeat that is non-resolvable under the conditions used to produce the virus vector. Further, an AAV terminal repeat can be modified so that resolution by the AAV Rep proteins is substantially reduced or eliminated. The non-resolvable terminal repeat can be any inverted repeat sequence that forms a hairpin structure and cannot be nicked by the AAV Rep proteins.

The inverted terminal repeats (ITR) are typically present in at least two copies in the AAV vector, typically flanking the expression cassette containing the nucleotide sequence(s) encoding an enzyme which has a DNase activity. The ITRs typically will be at the 5′ and 3′ ends of the nucleotide sequence(s) encoding an enzyme which has a DNase activity, but need not be contiguous thereto. The ITRs can be the same or different from each other. The term “terminal repeat” includes any viral terminal repeat and/or partially or completely synthetic sequences that form hairpin structures and function as an inverted terminal repeat, such as the “double-D sequence” as described in U.S. Pat. No. 5,478,745 to Samulski et al. An “AAV terminal repeat” may be from any AAV, including but not limited to serotypes 1, 2, 3, 4, 5, 6, 7, 8, 9, 10, 11, 12, Anc80, or any other AAV now known or later discovered. The AAV terminal repeat need not have a wild-type sequence (e.g., a wild-type sequence may be altered by insertion, deletion, truncation or missense mutations), as long as the terminal repeat mediates the desired functions, e.g., replication, nicking, virus packaging, integration, and/or provirus rescue, and the like. The vector construct can comprise one or more (e.g., two) AAV terminal repeats, which may be the same or different. Further, the one or more AAV terminal repeats can be from the same AAV serotype as the AAV capsid, or can be different. In particular embodiments, the vector construct comprises an AAV1, AAV2, AAV3, AAV4, AAV5, AAV6, AAV7, AAV8, AAV9, AAV10, AAV11, AAV12, and/or Anc80 terminal repeat.

Parvoviral ITR nucleotide sequences are typically palindromic sequences, comprising mostly complementary, symmetrically arranged sequences also referred to as “A,” “B,” and “C” regions. The ITR functions as an origin of replication, a site having a “cis” role in replication, i.e., being a recognition site for trans acting replication proteins such as e.g. Rep 78 (or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Two or another even number of regular ITRs can be used.

Functional ITR sequences are necessary for the replication, rescue and packaging of AAV virions. The ITR sequences may be wild type sequences or may have at least 80%, 85%, 90%, 95%, or 100% sequence identity with wild type sequences. The ITR sequences may be altered by for example in insertion, mutation, deletion or substitution of nucleotides, as long as they remain functional. In this context, functionality refers to the ability to direct packaging of the genome into the capsid shell and then allow for expression in the host cell to be transduced or target cell.

The AAV vector can comprise of single stranded or double stranded (self-complementary) DNA. The single stranded nucleic acid molecule is either sense or antisense strand, as both polarities are equally capable of gene expression. The AAV vector may further comprise a marker or reporter gene, such as a gene for example encoding an antibiotic resistance gene, a fluorescent protein (e.g., GFP) or a gene encoding a chemically, enzymatically or otherwise detectable and/or selectable product (e.g., lacZ, aph, etc.) known in the art.

AAV expression vectors may comprise a nucleic acid that may include a secretory signal sequence allowing secretion of the encoded enzyme which has a DNase activity from the transduced cell. Non-limiting examples of such secretory signal sequences include, e.g., DNase I secretory signal sequence, IL2 secretory signal sequence, albumin secretory signal sequence, β-glucuronidase secretory signal sequence, alkaline protease secretory signal sequence, and fibronectin secretory signal sequence.

In some embodiments, an AAV9-based or an Anc80-based vector is used as the expression vector. The AAV9 and Anc80 vectors are particularly suited for CNS targeting and expression. For example, both AAV9 and Anc80 vectors can transfect CNS cells with greater efficiency as compared to an AAV2 vector. Both AAV9 and Anc80 also induce lower amounts of neutralizing antibodies than some of the other AAV vectors.

An AAV9 vector or an Anc80 vector comprising a nucleotide encoding for an enzyme which has a DNase activity can be administered intracerebral (e.g., via direct organ injection) or systemically, e.g., by intravenous, intraarterial, intracerebral, intracerebroventricular (i.c.v.), intraparenchymal injections, intrastriatal, intraspinal, intrathecal, subarachnoid injections, with the AAV9 or Anc80 vector effective to transfect CNS cells and mediate effective production of the encoded enzyme which has a DNase activity and its secretion. In some embodiments, an Anc80 capsid protein (e.g., Anc80 VP1 capsid protein comprising the sequence SEQ ID NO: 3 or SEQ ID NO: 9, or an Anc80L65 VP1 capsid protein comprising the sequence of SEQ ID NO:34, or a variant Anc80L65 VP1 capsid protein comprising the sequence of SEQ ID NO: 35) is encoded by a nucleotide sequence in the expression vector. In some embodiments, an AAV9 capsid protein (e.g., AAV9 VP1 or AAV8 VP3) is encoded by a nucleotide sequence in the expression vector. Peripheral administration of the AAV vectors of the invention may include systemic injections, such as, e.g., intramuscular, intravenous, intraperitoneal, intra-arterial, intracerebral, intracerebroventricular (i.c.v.), intraparenchymal injections, intrastriatal, intraspinal, intrathecal, subarachnoid injection

The desired doses of the DNase enzyme encoding AAV vectors of the invention may be easily adapted by the skilled artisan, e.g., depending on the disease condition, the subject, the treatment schedule, etc. In some embodiments, from 10⁵ to 10¹⁴ recombinant viral particles are administered per dose, for example, from 10⁶ to 10¹¹, from 10⁷ to 10¹¹, or from 10⁸ to 10¹⁶. In other embodiments, exemplary doses for achieving therapeutic effects may include titers of at least about 10⁵, 10⁶, 10⁷, 10⁸, 10⁹, 10¹⁰ or 10¹¹ recombinant viral particles or more.

The exogenous targeting sequence(s) may replace or substitute part or all of a major capsid subunit (e.g., VP3). As a further alternative, more than one exogenous targeting sequence, e.g., two, three, four, five or more sequences, may be introduced into the virion capsid. In alternative embodiments, insertions and substitutions within the minor capsid subunits (e.g., VP1 and VP2) may be undertaken. For AAV capsids, insertions or substitutions in VP2 or VP3 may be undertaken.

The native virion tropism may be reduced or abolished by insertion or substitution of the amino acid sequence. Alternatively, the insertion or substitution of the exogenous amino acid sequence may target the virion to a particular cell type(s). The exogenous targeting sequence may be any amino acid sequence encoding a protein or peptide that alters the tropism of the virion. In particular embodiments, the targeting peptide or protein may be naturally occurring or, alternately, completely or partially synthetic. Exemplary peptides and proteins include ligands and other peptides that bind to cell surface receptors present in nervous system cells include ligands capable of binding the Sr-B 1 receptor for apoliprotein E, galactose- and lactose-specific lectins, low density lipoprotein receptor ligands, asialoglycoprotein (galactose-terminal) ligands and the like.

Alternatively, the exogenous targeting sequence may be an antibody or an antigen-recognizing moiety thereof. The term “antibody” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. Also encompassed by the term “antibody” are bispecific or “bridging” antibodies as known by those skilled in the art. Antibody fragments within the scope of the present invention include, for example, Fab, F(ab′)₂, and Fc fragments, and the corresponding fragments obtained from antibodies other than IgG. Such fragments may be produced by known techniques. The exogenous amino acid sequence inserted into the virion capsid may be one that facilitates purification or detection of the virion. For example, the exogenous amino acid sequence may include a poly-histidine sequence that is useful for purifying the virion over a nickel column, as is known to those skilled in the art or an antigenic peptide or protein that may be employed to purify the virion by standard immunopurification techniques. Alternatively, the amino acid sequence may encode a receptor ligand or any other peptide or protein that may be used to purify the modified virion by affinity purification or any other techniques known in the art (e.g., purification techniques based on differential size, density, charge, or isoelectric point, ion-exchange chromatography, or peptide chromatography).

Alternatively, the exogenous targeting sequence may be an antibody or an antigen-recognizing moiety thereof. The term “antibody” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. Also encompassed by the term “antibody” are bispecific or “bridging” antibodies as known by those skilled in the art. Antibody fragments within the scope of the present invention include, for example, Fab, F(ab′)₂, and Fc fragments, and the corresponding fragments obtained from antibodies other than IgG. Such fragments may be produced by known techniques.

The exogenous amino acid sequence inserted into the virion capsid may be one that facilitates purification or detection of the virion. According to this aspect of the invention, it is not necessary that the exogenous amino acid sequence also alters the virion of the modified parvovirus. For example, the exogenous amino acid sequence may include a poly-histidine sequence that is useful for purifying the virion over a nickel column, as is known to those skilled in the art or an antigenic peptide or protein that may be employed to purify the virion by standard immunopurification techniques. Alternatively, the amino acid sequence may encode a receptor ligand or any other peptide or protein that may be used to purify the modified virion by affinity purification or any other techniques known in the art (e.g., purification techniques based on differential size, density, charge, or isoelectric point, ion-exchange chromatography, or peptide chromatography).

### Adenovirus Vectors

Adenovirus genome is a linear, 36-Kb double-stranded DNA containing multiple, heavily spliced transcripts. At either end of the genome are inverted terminal repeats (ITRs). Genes are divided into early (E1-E4) and late (L1 -L5) transcripts.

In some embodiments, recombinant adenovirus vectors have two genes deleted: E1 and E3 (dE1/E3). Deletion of E1 makes the viral vector replication-deficient. E1 can be supplied by the adenovirus packaging cell lines (e.g., T 293 or 911). E3 is involved in evading host immunity and is not essential for virus production. Deletion of E1 and E3 results in a transgene packaging capacity of >8 Kb. In some embodiments, Constructs contain left and right arms to facilitate homologous recombination of the transgene into the adenoviral plasmid.

Any of the accepted human adenovirus types may be used, such as, e.g., an adenoviral vector based on Ad5. Ad5-based vectors use the Coxsackie-Adenovirus Receptor (CAR) to enter cells. In some embodiments, human adenovirus Type 5 (dE1/E3) is used as a vector. Most adenovirus vectors are engineered such that a transgene replaces the Ad E1a, E1b, and/or E3 genes; subsequently the replication defective vector is propagated in human 293 cells that supply deleted gene function in trans. Adenovirus vectors can transduce multiple types of tissues in vivo, including nondividing, differentiated cells such as those found in the nervous system. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

### Naked DNA Delivery

In some embodiments, an expression vector comprising a sequence encoding an enzyme which has a DNase activity is administered to the subject as a naked DNA. Such naked DNA can be delivered, e.g., into a CSF or brain.

### Enveloped Viral Vectors

In various embodiments of the invention, an enveloped viral particle can be used for nervous system delivery of a nucleic acid encoding an enzyme which has a DNase activity.

Non-limiting examples of useful enveloped viral vectors include, e.g., retroviruses (e.g., rous sarcoma virus, human and bovine T-cell leukemia virus (HTLV and BLV), lentiviruses (e.g., human and simian immunodeficiency viruses (HIV and SIV), Mason-Pfizer monkey virus)), foamy viruses (e.g., Human Foamy Virus (HFV)), herpes viruses (herpes simplex virus (HSV), varicella-zoster virus, VZVEBV, HCMV, HHV), hantaviruses, pox viruses (e.g., vertebrate and avian poxviruses, vaccinia viruses), orthomyxoviruses (e.g., influenza A, influenza B, influenza C viruses), paramyxoviruses (e.g., parainfluenza virus), respiratory syncytial virus, Sendai virus, mumps virus, measles and measles-like viruses), rhabdoviruses (e.g., vesicular stomatitis virus, rubella virus, rabies virus), coronaviruses (e.g., SARS, MERS), flaviviruses (e.g., Marburg virus, Reston virus, Ebola virus), alphaviruses (e.g., Sindbis virus), bunyaviruses, arenaviruses (e.g., LCMV, GTOV, JUNV, LASV, LUJV, MACV, SABV, WWAV), iridoviruses, and hepadnaviruses.

In some embodiments, the viral particle described herein is derived from a virus of the family Retroviridae. In one specific embodiment, the viral particle described herein is a retroviral particle. In another specific embodiment, the viral particle described herein is a lentiviral particle. Compared to other gene transfer systems, lentiviral and retroviral vectors offer a wide range of advantages, including their ability to transduce a variety of cell types, to stably integrate transferred genetic material into the genome of the targeted host cell, and to express the transduced gene at significant levels. Vectors derived from the gamma-retroviruses, for example, the murine leukemia virus (MLV), have been used in clinical gene therapy trials (Ross et al., Hum. Gen Ther. 7:1781-1790, 1996).

In one specific embodiment, the at least one viral element associated with a nucleotide of interest is a retroviral element. In another specific embodiment, the at least one viral element associated with a nucleotide of interest is a lentiviral element. In one specific embodiment, the at least one viral element associated with a nucleotide of interest is a Psi (ψ) packaging signal.

In one specific embodiment, the lentiviral particle does not contain gp120 surface envelope protein and/or gp41 transmembrane envelope protein. In another specific embodiment, the lentiviral particle contains a mutant gp120 surface envelope protein and/or a mutant gp41 transmembrane envelope protein.

In some embodiments, the viral particle described herein comprises components from a virus selected from the group consisting of Human Immunodeficiency Virus (e.g., HIV-1 or HIV-2), Bovine Immunodeficiency Virus (BIV), Feline Immunodeficiency Virus (FIV), Simian Immunodeficiency Virus (SIV), Equine Infectious Anemia Virus (EIAV), Murine Stem Cell Virus (MSCV), Murine Leukemia Virus (MLV), Avian leukosis virus (ALV), Feline leukemia virus (FLV), Bovine leukemia virus (BLV), Human T-lymphotropic virus (HTLV), feline sarcoma virus, avian reticuloendotheliosis virus, caprine arthritis encephalitis virus (CAEV), and Visna-Maedi virus (VMV).

In some embodiments, the viral particles described herein are replication deficient and only contain an incomplete genome of the virus from which they are derived. For example, in some embodiments, the lentiviral and retroviral particles do not comprise the genetic information of the gag, env, or pol genes (which may be involved in the assembly of the viral particle), which is a known minimal requirement for successful replication of a lentivirus or retrovirus. In these cases, the minimal set of viral proteins needed to assemble the vector particle are provided in trans by means of a packaging cell line. In one specific embodiment, env, tat, vif, vpu and nef genes are lacking in lentiviral particles derived from HIV-1 and are provided in trans or are made inactive by the use of frame shift mutation(s).

In some embodiments, the RNA molecule incorporated into the lentiviral or retroviral particles (and encoding an enzyme which has a DNase activity) comprises the psi packaging signal and LTRs. To achieve expression of the nucleotide sequence encoding an enzyme which has a DNase activity in nervous system cells, such sequence is usually placed under the control of a nervous system-specific promoter. Non-limiting examples of useful nervous system-specific promoters include, e.g., microglia-specific promoters (e.g., F4/80, CD68, TMEM119, CX3CR1, CMV, and Ibal promoters), myeloid-specific promoters (e.g., TTR, CD11b, and c-fes promoters), neuron-specific promoters (e.g., CMV, NSE, synapsin [SynI, SynII], CamKII , or α-CaMKII, GfaABC1D-dYFP, and VGLUT1 promoters), and other neural and glial cell (e.g., oligodendrocytes, astrocytes) type-specific promoters (e.g., glial fibrillary acidic protein [GFAP] promoter).

In some embodiments of lentiviral and retroviral particles, the RNA molecule together with the gag and pol encoded proteins, provided in trans by the packaging cell line, are then assembled into the vector particles, which then infect cells, reverse-transcribe the RNA molecule that comprises a nucleotide sequence encoding an enzyme which has a DNase activity under the control of a promoter, and either integrate said genetic information into the genome of the target cells or remain episomal (if one or more of the components required for integration are disrupted). If the genetic information for the gag and pol encoded proteins is not present on the transduced RNA molecule, the vector particles are replication deficient, i.e., no new generation of said vector particles will thus be generated by the transduced cell, thus ensuring safety in clinical applications.

In some embodiments of any of the above methods, the one or more viral elements encodes Human Immunodeficiency Virus (HIV) component(s), Bovine Immunodeficiency Virus (BIV) component(s), Feline Immunodeficiency Virus (FIV) component(s), Simian Immunodeficiency Virus (SIV) component(s), Equine Infectious Anemia Virus (EIAV) component(s), Murine Stem Cell Virus (MSCV) component(s), Murine Leukemia Virus (MLV) component(s), Avian leukosis virus (ALV) component(s), Feline leukemia virus (FLV) component(s), Bovine leukemia virus (BLV) component(s), Human T-lymphotropic virus (HTLV) component(s), feline sarcoma virus component(s), avian reticuloendotheliosis virus component(s), caprine arthritis encephalitis virus (CAEV) component(s), and/or Visna-Maedi virus (VMV) component(s).

In conjunction with the viral particles described herein, described herein are methods for detecting and/or isolating cells. In some embodiments, such method uses viral particles comprising a selectable marker (e.g., neomycin resistance) and/or a reporter (e.g., GFP or eGFP) as a nucleotide sequence of interest allowing target cells to be selected using, e.g., selection compound exposure or fluorescent activated cell sorting (FACS).

In some embodiments, the retroviral vectors described herein are derived from murine leukemia virus (MLV). Retroviral vectors encoding MLV are widely available to those skilled in the art, such as PINCO (Grignani et al., 1998) or the pBabe vector series (Morgenstern and Land, 1990).

In some embodiments, the lentiviral particles described herein are derived from a lentivirus such as human immunodeficiency virus (HIV). Suitable vectors encoding HIV and other useful viruses can be readily identified and/or prepared by the skilled person.

In one specific embodiment, the transfer vector comprises: a cytomegalovirus (CMV) enhancer/promoter sequence; the R and U5 sequences from the HIV 5′ LTR; the HIV-1 flap signal; an internal enhancer; an internal promoter; a nucleotide sequence of interest; the woodchuck hepatitis virus responsive element; a tRNA amber suppressor sequence; a U3 element with a deletion of its enhancer sequence; the chicken β-globin insulator; and the R and U5 sequences of the 3′ HIV LTR.

The invention also contemplates enveloped viral vectors comprising a heterologous targeting sequence or molecule inserted or substituted into the native envelope. The heterologous targeting sequence or molecule may confer an altered tropism towards neurons or increase the efficiency of nervous system delivery of the vector.

### Methods and Regimens for Administering Expression Vectors Encoding DNase Enzyme

In the methods of the invention, expression vectors comprising a nucleic acid encoding and enzyme which has a DNase activity can be administered to a patient at one time or over a series of treatments; once or several times per day.

The effective amount of expression vector to be administered will vary from patient to patient. Accordingly, effective amounts are best determined by the physician administering the compositions and appropriate dosages can be determined readily by one of ordinary skill in the art. Analysis of the blood (serum, plasma) or tissue levels of the vector-encoded enzyme which has a DNase activity and comparison to the initial level prior to administration can determine whether the amount being administered is too low, within the right range or too high. Suitable regimes for initial and subsequent administrations are also variable, but are typified by an initial administration followed by subsequent administrations if necessary. Subsequent administrations may be administered at variable intervals, ranging from daily to weekly to monthly to annually to every several years.

Doses of an expression vector encoding for an enzyme which has a DNase activity depend on the type of condition to be treated, the severity and course of these side effects, the patient's clinical history, discretion of the attending physician, and response to a prior treatment such as, e.g., chemotherapy, or radiation therapy, and DNase protein therapy. In some embodiments, the effective amount of expression vector is the amount which results in DNase protein level in CSF which is from 0.001 to 1 mg/L or from 2 to 2000 (KU)/L or from 0.5 to 100 mg/L or from 1000 to 200000 Kunitz units (KU)/L, from 0.5 to 50 mg/L or from 1000 to 100000 Kunitz units (KU)/L, from 1.5 to 50 mg/L or from 3000 to 100000 KU/L, from 10 to 50 mg/L or from 20000 to 100000 KU/L.

The administration of an expression vector, e.g., AAV- or viral-vector, encoding for DNase enzyme, and/or DNase enzyme, according to the methods of the invention can be performed by any suitable route, including, e.g., intravenous, intracerebral, intracerebroventricular (i.c.v.), intraparenchymal, intrastriatal, intracerebroventricular, intraspinal, intrathecal, subarachnoid administration.

### Cancer Treatment

In one aspect is provided a method for treating or ameliorating nervous system tumors in a subject suffering from a cancer, which method comprises administering to the subject a therapeutically effective amount of an expression vector encoding an enzyme which has a DNase activity (e.g., DNase I such as, e.g., human recombinant DNase I or bovine pancreatic DNase I), analogues of DNase I (such as, e.g., DNase X, DNase gamma, or DNAS1L2), DNase II (e.g., DNase II-alpha, DNase II-beta), phosphodiesterase I, lactoferrin, or acetylcholinesterase). In some embodiments, the DNase enzyme comprises the sequence set forth in SEQ ID NO: 1 (wild-type precursor sequence comprising the secretory signal sequence). In some embodiments, the DNase enzyme comprises the sequence set forth in SEQ ID NO: 2 (precursor mutant sequence comprising the secretory signal sequence and also comprising mutations weakening actin-mediated inhibition). In some embodiments, the DNase enzyme comprises the sequence set forth in SEQ ID NO: 4 (wild-type mature sequence without the secretory signal sequence). In some embodiments, the DNase enzyme comprises the sequence set forth in SEQ ID NO: 5 (mature mutant sequence without the secretory signal sequence and comprising mutations weakening actin-mediated inhibition). In some embodiments, the DNase enzyme is expressed under the control of a nervous system-specific promoter or a nervous system-specific enhancer element.

The amount and manner of administration of the expression vectors of the invention should be effective to treat a cancer, e.g., by one or more of reducing tumor size, reducing the rate of tumor growth, reducing the rate of metastasis, enhancing the therapeutic effects of chemotherapy and/or radiotherapy and/or radiosurgery, prolonging lifespan, reducing the rate of cancer- or treatment-associated side effects.

In some embodiments, the administration of an expression vector encoding an enzyme which has a DNase activity can be combined with administration of the same enzyme as protein (e.g., intravenously, intra-arterially, intracerebrally, intracerebroventricularly, intraparenchymally, intrastriatally, intracerebroventricularly, intraspinally, intrathecally, subarachnoidaly intramuscularly, intraperitoneally, enterally, etc.). The enzyme can be, e.g., any one of DNase enzymes (e.g., DNase I (e.g., human recombinant DNase 1 or bovine pancreatic DNase I), analogues of DNase I (such as, e.g., DNase X, DNase gamma, or DNAS1L2), DNase II (e.g., DNase II-alpha, DNase II-beta), phosphodiesterase I, lactoferrin, or acetylcholinesterase).

The methods of treatment using the vectors of the invention can be used in subjects suffering from a broad range of nervous system tumors. The present invention is particularly useful for treating “nervous system cancers”, which include, e.g., astrocytoma, gliomas, glioblastoma, ependymoma, medulloblastoma, CNS lymphoma, craniopharyngioma, glioblastoma, meningioma, pituitary carcinomas, pituitary adenoma, neurofibromatosis, embryonal tumors, tumors of the pineal region, tumors of meninges, choroid plexus tumors, neuronal and mixed neuronal-glial tumors, germ cell tumors, and nervous system metastatic diseases of any origin.

For combination cancer treatments, an expression vector, e.g., AAV vector or lentiviral vector or adenoviral vector encoding an enzyme which has a DNase activity can be administered before, with, or after a chemotherapeutic agent (or a radiation therapy, radiosurgery therapy, surgery).

In some embodiments, the methods of the invention are used to prevent a cancer in a patient, e.g. a patient with a high risk of predisposition to nervous system cancer.

In some embodiments, the methods of the invention are used to prevent a cancer following a positive result from a blood-based or CSF-based DNA screening test (e.g., liquid biopsy) for the detection of predisposition to a cancer.

In some embodiments, the methods of the invention are used to prevent a cancer following the evaluation of a tumor susceptibility gene. Examples of tumor susceptibility genes include, but are not limited to CCDC26, CDKN2BAS, RTEL1, TERT, ERCC1, ERCC2, ERCC5, BRCA2, IDH1/2, NF1, NF2, TSC1, TSC2, TP53, PTEN, CASP-9, CAMKK2, P2RX7, MSH6, PDTM25, KDR, VTI1A , ETFA, TMEM127, GSTT1, CHAF1A , RCC1, XRCC1, EME1, ATM, GLTSCR1, XRCC4, GLM2, PTEN, CDKN2A, CDKN2B, p14/ARF, XRCC3, MGMT, XRCC4, MMR, IDH1, ERBB2, CDKN2A, CCDC26, SUFU, NPAS2, CCDKN2A, PTCH2, CTNNB1, P21, RIC8A, CASP8, XRCC1, WRN, BRIP1, SMARCE1, MN1, and PDGFB.

The susceptibility gene can be evaluated by one or more of Next-generation sequencing, whole genome sequencing, exome sequencing, an ELISA-based method, and PCR amplification. Other methods can be used to evaluate the susceptibility gene as well.

In one embodiment of any of the methods of the invention, the subject is human.

### Amelioration of Toxicity of Cancer Chemotherapy or Radiation Therapy

In one aspect, the invention provides a method for preventing or ameliorating a toxicity, or a condition, associated with a cytostatic and/or cytotoxic chemotherapy in a subject suffering from a nervous system cancer and receiving or deemed to receive said chemotherapy, radiotherapy, radiosurgery, other physical methods associated nervous system cancer therapies (e.g., surgery or magnetic resonance imaging-related heating) which method comprises administering to the subject a therapeutically effective amount of an expression vector encoding for an enzyme which has a DNase activity (e.g., DNase I (e.g., human recombinant DNase I or bovine pancreatic DNase I), analogues of DNase I (such as, e.g., DNase X, DNase gamma, or DNAS1L2), DNase II (e.g., DNase II-alpha, DNase II-beta), phosphodiesterase I, lactoferrin, or acetylcholinesterase). In some embodiments, the DNase enzyme comprises the sequence set forth in SEQ ID NO: 1 (wild-type precursor sequence comprising the secretory signal sequence). In some embodiments, the DNase enzyme comprises the sequence set forth in SEQ ID NO: 2 (precursor mutant sequence comprising the secretory signal sequence and also comprising mutations weakening actin-mediated inhibition). In some embodiments, the DNase enzyme comprises the sequence set forth in SEQ ID NO: 4 (wild-type mature sequence without the secretory signal sequence). In some embodiments, the DNase enzyme comprises the sequence set forth in SEQ ID NO: 5 (mature mutant sequence without the secretory signal sequence and comprising mutations weakening actin-mediated inhibition). In some embodiments, the DNase enzyme is expressed under the control of a nervous system-specific promoter and/or a nervous system-specific enhancer element.

The amount and manner of administration of the expression vector is effective to ameliorate at least one side effect of said chemotherapy.

The condition associated with a cytostatic and/or cytotoxic chemotherapy, radiotherapy, radiosurgery can include, e.g., body weight loss, headaches, fatigue, nausea, vomiting, troubles with memory and speech, dementia, and a second cancer.

In some embodiments, the cancer is associated with an increased level of cfDNA in CSF or nervous system tissues, which level is higher than the control level (e.g., the level of cfDNA in CSF of a healthy age-matched individual or an average level of cfDNA in CSF or intestine of several healthy age-matched individuals).

In some embodiments, the effects of chemotherapy are associated with an increased level of cfDNA in CSF of the patient, which level is higher than the control level (e.g., the level of cfDNA in CSF of an age-matched individual with a similar cancer profile who does not receive chemotherapy, or an average level of cfDNA in blood or CSF or intestine of several age-matched individuals who have cancer but do not receive chemotherapy).

In a further aspect, the invention provides a method for increasing the efficacy of a cytostatic and/or cytotoxic chemotherapy and/or radiotherapy and/or immunotherapy and/or radiosurgery in a subject suffering from a cancer and receiving or deemed to receive said therapy, which method comprises administering to the subject a therapeutically effective amount of an expression vector (e.g., Anc80 or AAV8 AAV vector or lentiviral vector or adenoviral vector) encoding an enzyme which has a DNase activity (e.g., DNase I (e.g., human recombinant DNase I or bovine pancreatic DNase I), analogues of DNase I (such as, e.g., DNase X, DNase gamma, or DNAS1L2), DNase II (DNase II-alpha, DNase II-beta), phosphodiesterase I, lactoferrin, or acetylcholinesterase). The amount and manner of administration of the expression vector is effective to prevent or ameliorate at least one side effect of said chemotherapy, and to prevent or ameliorate toxicity associated with said chemotherapy. In some embodiments, any one of DNase enzyme (e.g., DNase I (e.g., human recombinant DNase I or bovine pancreatic DNase I), analogues of DNase I (such as, e.g., DNase X, DNase gamma, or DNAS1L2), DNase II (e.g., DNase II-alpha, DNase II-beta), phosphodiesterase I, lactoferrin, or acetylcholinesterase) is also administered, e.g., intravenously.

The expression vectors of the invention can be also used to ameliorate toxicity and increase efficacy of various types of radiation therapy, including, for example, stereotactic radiotherapy, radiosurgery, brachytherapy/sealed source radiation therapy, systemic radioisotope therapy/unsealed source radiotherapy, conventional radiation therapy, 3-dimensional conformal radiation therapy, intensity modulated radiation therapy, proton therapy, fractionated stereotactic radiation therapy. Non-limiting examples of side effects of radiotherapy which can be prevented or ameliorated by administering an expression vector according to the methods of the invention include, for example, skin irritation or damage, fatigue, nausea, vomiting, dementia, stroke, memory and speech disorder, bowel damage, memory loss, infertility, and a second cancer.

The expression vectors of the invention can be used to increase efficacy of cancer immunotherapy, including, for example, antibodies (naked monoclonal antibodies, conjugated monoclonal antibodies, labeled antibodies, bispecific monoclonal antibodies), cancer vaccines, Immune checkpoint inhibitors (Drugs that target PD-1 or PD-L1, CTLA-4), Non-specific cancer immunotherapies and adjuvants, CAR-T therapies.

### Neurodegeneration Treatment

In one aspect, the invention provides a method for preventing or treating or ameliorating neurodegeneration. The term “neurodegeneration” is used herein to refer to a separate clinical pathological condition with progressive loss of structure and/or function of neurons, including death of neurons, predominantly but not limited to the protein misfolding. The neurodegeneration can be primary or secondary.

In some embodiments, the neurodegeneration is associated with an increased level of microbial and/or viral cfDNA in blood or cerebrospinal fluid (CSF) or intestine of a patient, which level is higher than the control level (e.g., the level of cfDNA in blood or CSF or intestine of a healthy age-matched individual or an average level of cfDNA in blood or cerebrospinal fluid or intestine of several healthy age-matched individuals). In one embodiment, the neurodegeneration is associated with a neurodegenerative disorder. Non-limiting examples of encompassed neurodegenerative disorders include, e.g., Alzheimer's disease (e.g., late-onset Alzheimer's disease), Mild Cognitive Impairment (MCI), Parkinson's disease, Amyotrophic Lateral Sclerosis (ALS), and Huntington's disease (HD), prion-caused diseases, progressive supranuclear palsy (PSP), progressive supranuclear palsy (Steel-Richardson-Olszewski), corticobasal degeneration (CBD), chronic traumatic encephalopathy (CTE), multiple system atrophy (MSA), agyrophilic grain disease (AGD), Pick disease (PiD), frontotemporal dementia (FTD), frontotemporal dementia with parkinsonism-17 (FTDP-17), multiple sclerosis, CADASIL Syndrome, ankylosing spondylitis, Dentatorubro-pallido-Luysian atrophy, Kennedy disease, Friedreich's ataxia, cerebellar ataxia, spinocerebellar ataxia, Lewy body dementia, vascular dementias, familial Danish dementia, familial British dementia, spinal muscular atrophy, olivopontocerebellar atrophy (OPCA), senile dementia of the Alzheimer type, corticodentatonigral degeneration, Hallervorden-Spatz disease, progressive familial myoclonic epilepsy, striatonigral degeneration, torsion dystonia (e.g., torsion spasm; dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia and related disorders), Shy-Drager syndrome, primary lateral sclerosis, hereditary spastic paraplegia, peroneal muscular atrophy (Charcot-Marie-Tooth), hypertrophic interstitial polyneuropathy (Dejerine-Sottas), chronic progressive neuropathy, pigmentary degeneration of the retina (retinitis pigmentosa), and hereditary optic atrophy (Leber's disease), secondary neurodegeneration caused by necrosis (e.g., destruction of neurons by neoplasm, edema, hemorrhage, stroke, trauma, immune attack, hypoxia, poisoning, metabolic defects, and infections), and various taupathies (e.g., Primary age-related tauopathy (PART)/Neurofibrillary tangle-predominant senile dementia, with NFTs similar to AD, but without plaques, CTE, Lytico-Bodig disease, Ganglioglioma and gangliocytoma, Meningioangiomatosis, Postencephalitic parkinsonism, Subacute sclerosing panencephalitis). In some one embodiments, the neurodegeneration is associated with a nervous system dysfunction such as, e.g., schizophrenia or bipolar disorder, depressive disorder, autism, autism spectrum disorders, Chronic Fatigue Syndrome, Obsessive-Compulsive Disorder, generalized anxiety disorder (GAD), major depressive disorder (MDD), or social anxiety disorder (SAD). In some embodiments, the neurodegenerative disorder is caused by, secondary to, or associated with, amyloidosis. In some embodiments, the neurodegenerative disorder is a protein-misfolding associated disease. In some embodiments, the neurodegenerative disorder is caused by, secondary to, or associated with diabetes, rheumatoid arthritis, systemic lupus erythematosus (SLE), gout, metabolic syndrome, an amyloidosis (e.g., hereditary cerebral hemorrhage with amyloidosis, primary systemic amyloidosis, secondary systemic amyloidosis, serum amyloidosis, senile systemic amyloidosis, hemodialysis-related amyloidosis, Finnish hereditary systemic amyloidosis, Atrial amyloidosis, Lysozyme systemic amyloidosis, Insulin-related amyloidosis, Fibrinogen a-chain amyloidosis), asthma, or prion disease.

In some embodiments, the neurodegeneration is caused by, or is secondary to, or is associated with the formation of a misfolded protein due to the presence of microbial (e.g., bacterial, fungal, intracellular or extracellular parasites) and/or viral (e.g., bacteriophages, eukaryotic viruses) DNA.

In some embodiments, the neurodegeneration is caused by, or is secondary to, or is associated with the formation of a misfolded protein due to the presence of bacterial or viral DNA that is released inside human cells of nervous system (e.g. glial cells, neurons) DNA.

In some embodiments, the neurodegeneration is caused by, or is secondary to, or associated with the formation of a misfolded protein due to the presence of bacterial or viral DNA that is released inside human cells of nervous system (e.g. glial cells, neurons) DNA without of elevation of DNA level in CSF compared to healthy controls.

In some embodiments, the neurodegeneration is caused by, or is secondary to, or is associated with the formation of a misfolded protein including, but not limited to, β-amyloid, Tau protein, α-synuclein, SOD1, TDP-43, IAPP, ADan, ABri, Fused in sarcoma (FUS) protein, Notch3, Glial fibrillary acidic protein, Seipin, Transthyretin, Serpins, Apolipoproteins, Amyloid f3 peptide, Lactoferrin, and Galectin-7 Corneodesmosin.

In some embodiments, the methods of the invention can be used to treat or prevent development of a neurodegenerative disease or another disease associated with protein misfolding following the evaluation of the presence of, or activity of, a disease-associated susceptibility gene. Examples of disease-associated susceptibility genes, include, but are not limited to, ADAR1, MDA5 (IFIH1), RNase H subunits, SamHD1, TREX, TBK1, Optineurin, P62 (sequestosome 1), Progranulin, TDP43, FUS, VCP, CHMP2B, Profilin-1, Amyloid-β, Tau, α-synuclein, PINK, Parkin, LRRK2, DJ-1, GBA, ATPA13A2, EXOSCIII, TSEN2, TBC1D23, Risk-factor alleles, PLCG2, TREM2, APOE, TOMM40, IL-33, Glucocerebrosidase, Ataxin2 , C9orf72, SOD1, and FUS. The susceptibility gene can be evaluated, e.g., by one or more of next-generation sequencing (NGS), whole genome sequencing (WGS), exome sequencing, ELISA-based methods, and PCR amplification. Other methods can be used to evaluate the susceptibility gene as well.

In one embodiment of the above methods, prevention or treatment of the misfolded protein aggregate formation in mammalian biological fluids and tissues is achieved by the use of expression vectors disclosed herein.

As described in Int. Pat. Appl. Pub. No. WO 2016/190780, cfDNA from the intestine, blood and CSF of patients suffering from neurodegeneration causes neuronal cell death and apoptosis, and treatment with recombinant DNase protein destroying such cfDNA improves the nervous system function in these patients. As shown in the Examples section below, the use of the vectors of the invention for nervous system-specific expression of DNase, provides a further treatment improvement as compared to DNase protein administration.

The assessment of treatment efficacy for neurodegeneration and neuroinflammation can be performed using any methods known in the art, e.g., according to widely accepted clinical diagnostic criteria of cognitive decline such as MMSE, PANSS, physical function, and/or functional tasks (see, e.g., Holmes et al., (1999) The British Journal of Psychiatry, 174(1), 45-50; Os et al., (2006) Acta Psychiatrica Scandinavica, 113(2), 91-95; O'Shea et al., (2002) Physical therapy, 82(9), 888-897; Rochester et al., Arch. Phys. Med. Rehabil. (2004) 85(10), 1578-1585).

In some embodiments of the invention, the neurodegeneration is caused by, or is aggravated by an increased blood-brain barrier permeability leading to an increased level and translocation of viruses, fungi, bacteria (including prokaryotes and archea), intracellular or extracellular parasites, and/or microbial and/or viral cfDNA to the CSF and tissues of the nervous system. Such cfDNA may lead to protein misfolding and neurodegenerative diseases as well as to inflammasome development and neuroinflammation.

In some embodiments, the invention provides a method for treating neurodegeneration in a subject suffering from neurodegeneration, which method comprises administering to the subject a therapeutically effective amount of an expression vector encoding an enzyme which has a DNase activity (e.g., DNase I [e.g., human recombinant DNase I or bovine pancreatic DNase I], analogues of DNase I [such as, e.g., DNase X, DNase gamma, or DNAS1L2], DNase II [e.g., DNase II-alpha, DNase II-beta], phosphodiesterase I, lactoferrin, or acetylcholinesterase). The amount and manner of administration of the expression vector (e.g., AAV vector or lentiviral vector or adenoviral vector or liposome or naked DNA) should be effective to treat one or more symptoms or effects from such neurodegeneration.

Without wishing to be bound by any theory, it can be hypothesized that after a nucleic acid encoding an enzyme which has a DNase activity is delivered to the nervous system using the vectors and methods of the invention, the enzyme becomes produced by cells in nervous system and is secreted into the CSF, where the enzyme efficiently degrades the cfDNA present therein.

In some embodiments, the expression vectors encoding an enzyme which has a DNase activity can be administered in combination with other treatments useful for treatment of neurodegenerative diseases or other encompassed nervous system dysfunctions (e.g., bipolar disorder, migraine, schizophrenia, epilepsy). Non-limiting examples of such additional treatments include, e.g., transcranial magnetic stimulation, transcranial direct current stimulation, administration of Crenezumab, Solanezumab, CAD106, non-steroidal anti-inflammatory drugs, caffein A2A receptor antagonists, CERE-120 (adeno-associated virus serotype 2-neurturin), levodopa, amantadine, donepezil, hidergine, benztropine, biperiden, bromocriptine, carbidopa, entacapone, edaravone, etanercept. entacapone, galanthamine, laquinimod, memantine, pramipexole, peroglide, pramipexole pramiperoxole, prodopidine, procyclidine, rasagiline, riluzole, radicava, rivastigmine, ropinirole, rotigotine, selegiline, tacrine, tetrabenazine, tolcapone, trihexyphenidyl, gantenerumab, solanezumab, vitamin e; or administration of one or more compounds with a non-limiting example of being selected from the group consisting of: cognitive-enhancing agents, drugs for behavioral symptoms, disease-modifying therapies , drugs targeting amyloid-related mechanisms, drugs targeting tau -related mechanisms, histone acetyltransferase activators, cyclin-dependent protein kinase 5 inhibitors, neurotrophin mimetics, semaphorin-4D blockers, microsomal prostaglandin E synthase-1 inhibitors, and N-methyl-d-aspartate (NMDA) receptor inhibitors anti-amyloid drugs, neurotransmitter based drugs, gene silencing therapies, gene editing therapies, anti-inflammatory therapies, immunomodulators, axon regeneration, neuroprotection, sigma-1 receptor (S1R) and muscarinic receptor agonist, proteostasis, protein misfolding modulators, Neuroprotection, anitoxidants, SOD1-lowering therapy, SOD1 Metalation, mitochondrial bioenergetics, nuclear export blockers, misfolded proteins clearance, unfolded protein response (UPR), IRE1 alpha inhibitors, immunotherapy, with different mechanisms of action, not limited to monoclonal antibody, polyclonal antibody, mast cell stabilizers, sigma 1 receptor agonist; NMDA receptor antagonist, TNF alfa inhibitors, BACE1 inhibitor, 5-HT2A antagonist, dopamine receptor modulator, receptor antagonist, Inhibitors protein aggregation, modulator of GABA-A receptors.

### Pharmaceutical Compositions, Formulations and Dosage Forms

The AAV vectors disclosed herein may be administered in any suitable form, for instance, either as a liquid solution or suspension, as a solid form suitable for solution or suspension in liquid prior to injection. The vectors may be formulated with any appropriate and pharmaceutically acceptable excipient, carrier, adjuvant, diluent, etc. For instance, for injection, a suitable carrier or diluent may be an isotonic solution, a buffer, sterile and pyrogen-free water, or, for instance, a sterile and pyrogen-free phosphate-buffered saline solution. The expression vectors may be formulated in pharmaceutical compositions and dosage forms. Such pharmaceutical compositions and dosage forms can be formulated in any conventional manner using one or more physiologically acceptable carriers and/or excipients. The vector particles may be formulated for administration by, for example, injection. Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

Formulations also include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof In addition, the composition may contain minor amounts of auxiliary substances, such as, wetting or emulsifying agents, pH buffering agents, stabilizing agents or other reagents that enhance the effectiveness of the pharmaceutical composition.

The formulations used in the methods of the invention may conveniently be presented in unit dosage form and may be prepared by methods known in the art. The amount of active ingredients that can be combined with a carrier material to produce a single dosage form will vary depending upon the host being treated and the particular mode of administration. The amount of active ingredients that can be combined with a carrier material to produce a single dosage form will generally be that amount of the compound which produces a therapeutic effect.

Pharmaceutical compositions suitable for parenteral administration may further comprise one or more additional active ingredients (e.g., a DNase protein or another active compound) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Effect of DNA on Tau Protein Aggregation

In this Example, the inventors examined whether Tau fibril formation could be induced following treatment with bacterial and human DNA in Tau-protein misfolding cyclic amplification (PMCA) by monitoring the levels of Thioflavin T (ThT) fluorescence overtime.

Below are the materials and methods used in this Example.

Sources and Procedures for DNA Extraction. Extracellular DNA was extracted from the matrix of P. aeruginosa ATCC 27853, E. coli ATCC 25922, Escherichia coli 472217, Porphyromonas gingivalis, Borrelia burgdorferi; Tetzerella alzheimeri VT-16-1752, Tetzosporium hominis and Candida albicans. All bacterial strains were subcultured from freezer stocks onto Columbia agar plates (Oxoid, UK) and incubated at 37° C. for 48 hours, and the fungal strain was subcultured from freezer stocks onto Sabouraud dextrose agar (Oxoid, UK) and incubated at 30° C. for 48 hours. To extract the extracellular DNA, bacterial and fungal cells were separated from the matrix by centrifugation at 5000 g for 10 min at 4° C. The supernatant was aspirated and filtered through a 0.2-μm-pore-size cellulose acetate filter (Millipore Corporation, USA). Extracellular DNA was extracted using a DNeasy kit (Qiagen). Human genomic DNA (Roche Cat #11691112001) was purchased from Sigma (Sigma-Aldrich).

Tau Expression and Purification. For these studies full-length Tau containing four microtubule-binding domains and two N-terminal inserts was used, with its two cysteines residues (C291, C322) replaced by serines (Meyer et al. Amplification of Tau 542 Fibrils from Minute Quantities of Seeds. Biochemistry 53, 5804-5809 (2014)) to prevent formation of covalent dimers and aggregates. The plasmid encoding Tau40 was kindly provided by Dr Martin Margittai.

Expression and purification were performed using previously described procedures (Meyer et al. Amplification of Tau 542 Fibrils from Minute Quantities of Seeds. Biochemistry 53, 5804-5809 (2014)). Briefly, the plasmid was transformed into BL21 (DE3) Escherichia coli bacteria (New England BioLabs, catalog #C2527H) which were grown overnight in 30 μg/ml kanamycin Terrific Broth (TB) at 37° C. with agitation. The culture was then diluted 1:20 and grown until the optical density 600 nm reached 0.6. One mM isopropropyl-P-D-thiogalactopyranoside (IPTG) was added to induce protein expression, and then the cultures were grown at 37° C. with agitation for 6 hours. Bacteria were collected by centrifugation at 3,000×g and pellets stored frozen at −20° C. until lysis.

Pellets were thawed and resuspended in 20 mM PIPES pH 6.5, 500 mM NaCl, with protease inhibitor cocktail complete (Roche) and sonicated with a ½″ probe (S-4000, Misonix), then heated at 95° C. for 20 minutes. Lysates were centrifuged at 15,000×g for 20 min at 4° C. twice to remove cell debris. To precipitate proteins, ammonium sulfate (Sigma) was added at 55% w/v and incubated at room temperature for 1 hour with a magnetic stirrer. Precipitated protein was recovered by centrifugation at 15,000×g at room temperature, and pellets were stored at −20° C.

Tau protein was then purified by Cation Exchange Chromatography, pellets were dissolved in water (>18.2 MΩ cm) and then the solution was filtered through a 0.2 μm filter. The sample was applied to a Hitrap SP HP column and eluted in a linear salt gradient (50-1000 mM NaCl, 20 mM PIPES pH 6.5). The content of tau protein in the fractions was followed by sodium dodecyl sulfate polyacrylamide gel electrophoresis (SDS-PAGE) and Blue Coomasie staining. Fractions containing tau protein were pooled and dialyzed overnight 1:100 in 10 mM HEPES buffer pH 7.4, 100 mM NaCl.

Tau protein was concentrated in Amicon centrifugal filters with a molecular weight cut-off (MWCO) of 10 kDa and finally filtered through 100 kDa Amicon filter (Millipore) to remove pre-formed aggregates, aliquoted and stored at −80° C. Protein concentration was determined using bicinchoninic acid (BCA) protein Assay (Thermo Scientific).

Preparation of tau seeds. To prepare aggregated tau for seeding experiments, monomeric aggregate-free tau was incubated at a concentration of 50 μM, containing 25 μM heparin (Average molecular weight 18,000, Sigma) in 10 mM HEPES buffer pH 7.4, 100 mM NaCl for 5 days at 37° C. with constant shaking at 500 rpm in a thermomixer (Eppendorf). The formation of amyloid filaments was followed by Thioflavin T (ThT) fluorescence from samples taken from replicate tubes.

Tau filaments (2.3 mg/ml) were sonicated to prepare seeds (tau preformed fibrils (tau-PFF)) by diluting the filaments to 0.1 mg/ml in 10 mM HEPES buffer pH 7.4, 100 mM NaCl and sonicated inside an Eppendorf tube in a floating rack using a microplate horn sonicator (S-400 Misonix) with settings of 30 seconds of total sonication time with pulses of 1 s ON-1 s OFF at Amp 30.

Tau aggregation assay. Solutions of 22 μM aggregate-free tau40 in 100 mM HEPES pH 7.4, 100 mM NaCl (200 μl total volume) supplemented with 4.4 μM heparin were placed in opaque 96-wells plates and incubated alone or in the presence of tau-PFF used as seeds to trigger tau aggregation or in the presence of distinct concentrations of DNA. Samples were incubated in the presence of 10 μM Thioflavin T (ThT) and subjected to cyclic agitation (1 min at 500 rpm followed by 29 min without shaking) using an Eppendorf thermomixer, at a constant temperature of 20° C. At various time points, ThT fluorescence was measured in the plates at 485 nm after excitation at 435 nm using a plate spectrofluorometer.

The following probes were used to detect microbial or human DNA:
PA—extracellular matrix DNA of P. aeruginosa
TH—extracellular matrix DNA of Tetzosporium hominis
CA—extracellular matrix DNA of C. albicans
EC25—extracellular matrix DNA of E. coli ATCC 25922
EC47—extracellular matrix DNA of E. coli VT 472217
PG—extracellular matrix DNA of P. gingivalis
BB—extracellular matrix DNA of B. burgdorferi
Tetzerella alzheimeri VT-16-1752
Hum—human DNA

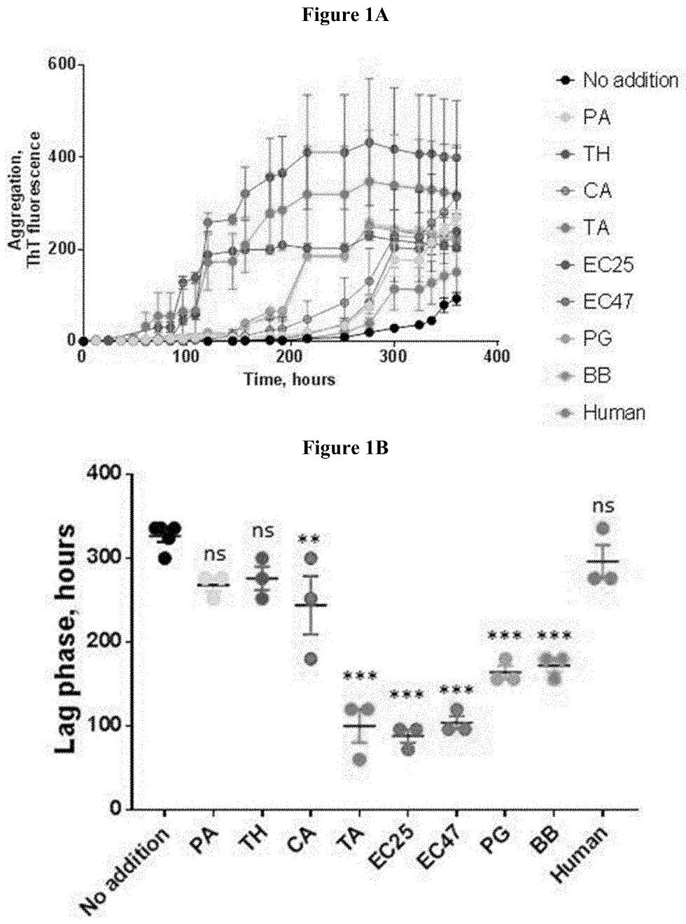

Effects of microbial and human DNA on Tau aggregation are shown in FIGS. 1, 2, and 3.

Using this tau aggregation assay, the inventors examined whether tau fibril formation could be promoted in the presence of extracellular DNA from various species including bacteria, yeast and human. For the experiments, monomeric tau was incubated with preparations containing 100 ng of DNA extracted from different bacterial species including Pseudomonas aeruginosa (PA), Tetzosporium hominis (TH), Tetzerella alzheimeri (TA), Escherichia coli ATCC 25922 (EC25), Escherichia coli ATCC 472217 (EC47), Porphyromonas gingivalis (PG), and Borrelia burgdorferi (BB). Tau was also incubated with the same amount of DNA extracted from Candida albicans (CA) and human samples. The results showed that DNA from various (but not all) bacterial species significantly promoted tau aggregation (FIG. 1A). Conversely, addition of eukaryotic DNA, such as from yeast or human cells, had a much lower effect in promoting tau aggregation. To compare the magnitude of the effect of these different DNA extracts on the kinetic of aggregation, the lag phase was measured, which was defined as the time at which aggregation begins and is experimentally determined as the time when ThT fluorescence reaches a value >40 fluorescent units (equivalent to ˜2-folds the background levels after subtraction of the blank). Comparisons of the lag phases indicate that the largest promoting effect (shorter lag phase) was obtained in the presence of Tetzerella alzheimeri, Escherichia coli ATCC 25922, and Escherichia coli 472217 (FIG. 1B). Interestingly, Tetzerella alzheimeri (VT-16-1752 gen.nov, sp.nov) is a new species which was isolated from the oral cavity of a patient with AD. Moderate promoting effect was observed with Porphyromonas gingivalis and Borrelia burgdorferi, and no significant effect was detectable for Pseudomonas aeruginosa and Tetzosporium hominis (FIG. 1B).

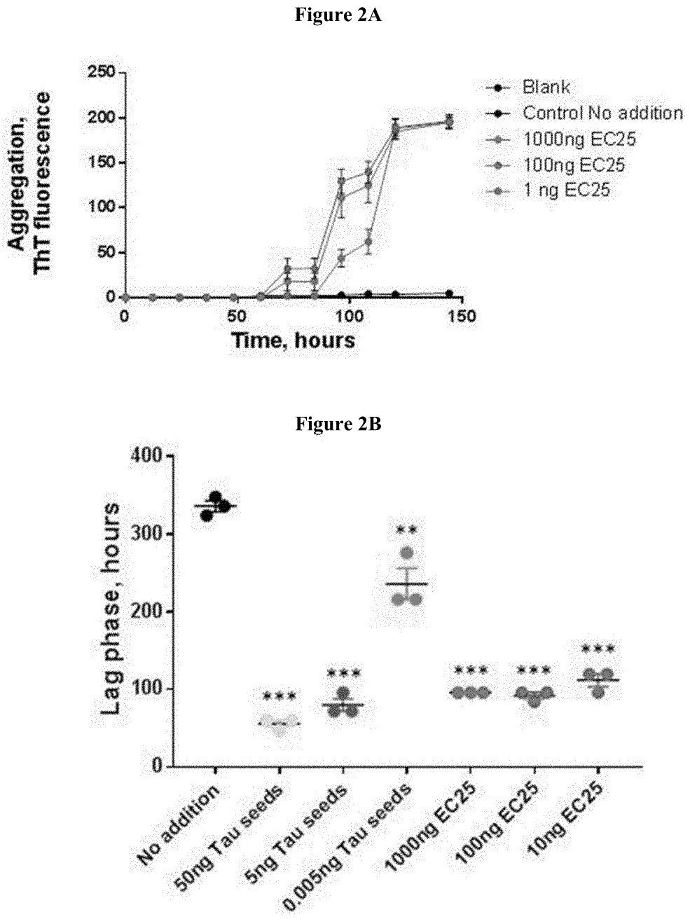

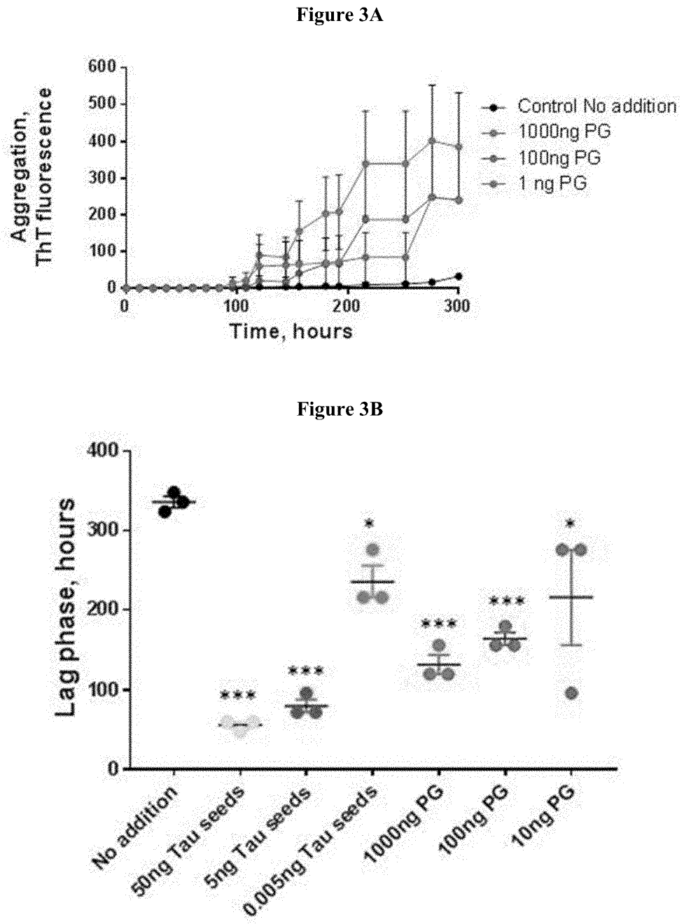

It was then determined whether the promoting activity of bacterial DNA is dose dependent. It was found that DNA of E. coli ATCC 25922 and P. gingivalis at concentrations of 1000 to 10 ng significantly accelerated Tau aggregation relative to controls. The promoting activity of E. coli ATCC 25922 (FIG. 2) and especially P. gingivalis (FIG. 3) was lower than that of tau seeds. A dose dependent effect was more clearly observed only for addition of P. gingivalis DNA, perhaps because of the higher efficiency of E. coli ATCC 25922, which may require lower concentrations to observe a dose-dependency.

### Example 2

### Effect of DNA of Different Origin on β-Amyloid Protein Misfolding

To study the role of DNA on β-amyloid misfolding, cyclic amplification of amyloid-β misfolding study was performed using in-vitro-produced oligomeric seeds.

Synthetic Aβ1-42 (2 mM) was mixed at 20° C. with constant shaking (100 rpm) in 100 mM Tris-HCl (pH 7.4). Amyloid-β aggregation was studied by the fluorescence emission of the amyloid-binding dye ThT (Soto, C., Castano, E. M., Frangione, B., and Inestrosa, N. C. (1995). The alpha-helical to beta-strand transition in the amino-terminal fragment of the amyloid beta-peptide modulates amyloid formation. J. Biol. Chem. 270, 3063-3067).

Seeding of Aβ aggregation was studied by addition of different quantities of DNA and further incubation for 300 hours.

Bacterial extracellular DNA was extracted from the matrix of E. coli 471277. Bacterial strains were subcultured from freezer stocks onto Columbia agar plates (Oxoid Ltd., London, England) and incubated at 37° C. for 48 hours. To extract extracellular DNA, the bacterial cells were separated from the matrix by centrifugation at 5,000 g for 10 min at 4° C. (Eppendorf Centrifuge 5810R). The supernatant was aspirated and filtered through a 0.2 μm cellulose acetate filter (Millipore Corporation, USA). eDNA was extracted using the DNeasy kit (Qiagen) as described by the manufacturer or the phenol-chloroform method.

Neutrophil extracellular traps (NETs) isolation. Fresh blood was collected to the heparinized tubes. Blood was washed with 5 ml of Phosphate Buffer Saline (PBS) without supplementation with calcium and magnesium to dilute the blood. 15 ml of lymphocyte separation media was added at the 25° C. Using a 18G needle, the diluted blood was carefully layered onto the Lymphocyte Separation Media to ensure that there was a lymphocyte separation media-blood interface. Next samples were centrifuged at 800×g for 30 min at 21° C. After erythrocytes and neutrophils were sedimented at the bottom, the supernatant was aspirated and discarded the top 2 layers (plasma and lymphocyte separation media). 20 ml of PBS and 20 ml of 6% Dextran solution were added and gently mixed. Neutrophil enriched supernatant was transferred into new tubes and centrifuged at 450×g at 4° C. for 5 min. Supernatant was discharged. The minor erythrocytes were lysed with lysing solution. After the next round of centrifugation at 450×g at 4° C. for 5 min, supernatant was discharged and washed with 5 ml PBS without calcium and magnesium and centrifuged again at 450×g at 4° C. The pellet was resuspended in 30 ml of cold 3% RPMI medium supplemented with 3% fetal bovine serum. Neutrophils were stimulated with 500 nM of phorbol myristate acetate (PMA) and incubated on a 150×25 mm flat tissue culture dish with 20 mm grid for 4 hour at 37° C. 5% CO₂ to stimulate NETosis. After 4 hour of stimulation, media was aspirated leaving the layer of NETs and neutrophils adhered at the bottom of the flask. Cold PBS (15 ml) was added to the flask, with pipetting to lift off all adherent material. Solution was collected and centrifuged for 10 min at 450×g at 4° C. Neutrophils and any remaining cells were sedimented, leaving a cell-free NET-rich supernatant. Supernatant was centrifuged at 18,000×g at 4° C. for 15 minutes to sediment DNA.

Cyclic amplification of amyloid-β misfolding was performed in six experimental groups:
Group 1—Blank (buffer)
Group 2—Control no seed
Group 3—DNA of E. coli 471277 10 ng/mL
Group 4—DNA of E. coli 471277 1 ng/mL
Group 5—DNA of E. coli 471277 0.1 ng/mL
Group 6—DNA of Human (NETs) 1 ng/mL

The results are summarized in Table 1. Aggregation was measured over time by the ThT binding to amyloid fibrils using a plate spectrofluorometer (excitation: 435 nm; emission: 485 nm)

TABLE 1
Average kinetics of aggregation of DNA

| Hours | Group 1 | Group 2 | Group 3 | Group 4 | Group 5 | Group 6 |
|---|---|---|---|---|---|---|
| 24 | 20.025 | 18.8 | 18.159 | 19.476 | 20.704 | 20.115 |
| 48 | 21.565 | 18.159 | 17.908 | 20.049 | 19.854 | 23.606 |
| 72 | 21.446 | 17.099 | 17.241 | 22.072 | 21.180 | 22.995 |
| 96 | 20.878 | 17.654 | 24.176 | 25.039 | 22.956 | 34.059 |
| 144 | 20.482 | 17.88 | 92.401 | 43.179 | 39.042 | 39.998 |
| 192 | 20.802 | 17.386 | 175.287 | 126.419 | 112.308 | 42.921 |
| 252 | 18.939 | 24.269 | 180.156 | 184.448 | 105.049 | 53.366 |
| 276 | 17.29 | 25.306 | 227.122 | 201.422 | 119.305 | 59.406 |
| 300 | 17.864 | 31.287 | 287.120 | 213.875 | 180.034 | 54.686 |

The result indicates that microbial cfDNAs significantly accelerated amyloid-β aggregation as compared to human DNA and control.

### Example 3

### Effects of Different Ways of DNase I Delivery on Parkinson's Disease Triggered by DNA

To study possible alterations of the efficacy of different ways of DNase I delivery for the treatment of neurodegenerative diseases, 12-week-old MitoPark mice were anesthetized by intraperitoneal injection of ketamine/xylazine in which Parkinson's disease was enhanced by stereotaxic injection targeting the dorsal striatum with 5 μL 1 ng (i) bacterial DNA, E. coli DNA (isolated as discussed above), or the same amount of (ii) human DNA.

Anc80L65-DNAse I hyperactive vector with a nervous system-specific promoter (Anc80L65-CMV promoter-hDNaseI (hyperactive)correct leader-WPRE.bGH) was stereotaxically injected targeting the dorsal striatum, and the ApoEHCR enhancer-hAAT promoter-hDNaseI (hyperactive)correct leader-WPRE Xinact (SEQ ID NO: 30) vector with a liver-specific promoter was intravenously injected 7 days after DNA injection.

Levels of dopamine metabolites 3,4-Dihydroxyphenylacetic acid (DOPAC) and homovanillic acid (HVA) in dorsal striata were measured with High Performance Liquid Chromatography (HPLC). For that dorsal striata were dissected and homogenized in 0.2 M perchloric acid supplemented with 100 μM EDTA-2Na (1:10, w/v) at 0° C. After 20 min, homogenates were centrifuged for 15 min at 4,000 g at 4° C. The supernatant was mixed with 0.4 M sodium acetate buffer (pH 3.0; 1:2, v/v) and filtered through a 0.22 μm milipore filter (5 min, 12,000 g at 4° C.). Dophamine and its metabolites DOPAC, HVA were determined by HPLC as described previously (Luk et al., Science 16 Nov. 2012: Vol. 338, Issue 6109, pp. 949-953, DOI: 10.1126/science.1227157).

Animals were allocated to the following experimental groups (n=5 per group) and received the treatments accordingly.
Group 1: Control animals were C57BL6/C3H age matched mice
Group 2: MitoPark mice intracerebrally injected with E. coli DNA 5 μL 1 ng+(7 days after the DNA injection) intraspinally injected with sterile saline.
Group 3: MitoPark mice intracerebrally injected with E. coli DNA 5 μL 1 ng+(7 days after the DNA injection) intraspinally injected with nervous system-specific Anc80L65-DNAse I hyperactive (Anc80L65-CMV-hDNaseI (hyperactive)correct leader-WPRE.bGH).
Group 4: MitoPark mice intracerebrally injected with E. coli DNA 5 μL 1 ng+(7 days after the DNA injection) iv injected ApoEHCR enhancer-hAAT promoter-hDNaseI (hyperactive)correct leader-WPRE Xinact.
Group 5: MitoPark mice intracerebrally injected with E. coli DNA 5 μL 1 ng+(7 days after the DNA injection) intraperitoneally (ip) injected DNase I protein (Genentech) 30000 Kunitz Units/kg/day, 2 times a day.
Group 6: MitoPark mice intracerebrally injected with human DNA 5 μL 1 ng+(7 days after the DNA injection) intraspinally injected with sterile saline.
Group 7: MitoPark mice intracerebrally injected with human DNA 5 μL1 ng+(7 days after the DNA injection) intraspinally injected with nervous system-specific Anc80L65-DNAse I hyperactive (Anc80L65-CMV-hDNaseI (hyperactive)correct leader-WPRE.bGH).
Group 8: MitoPark mice intracerebrally injected with human DNA 5 μL 1 ng+(7 days after the DNA injection) iv injected ApoEHCR enhancer-hAAT promoter-hDNaseI (hyperactive)correct leader-WPRE Xinact.
Group 9: MitoPark mice intracerebrally injected with human DNA 5 μL 1 ng+(7 days after the DNA injection) ip injected DNase I 30000 Kunitz Units/kg/day 2 times a day.

After 8 weeks animals were addressed for the loss of dopamine metabolites in the striatum. The results are summarized in Table 2. Data show mean+/−standard deviation (SD) (N=5 animals per group).

TABLE 2
Levels of dopamine metabolites, DOPAC
and HVA, relative to untreated control
(metabolite levels were determined by HPLC)

| Group | DOPAC | HVA |
|---|---|---|
| 1 | 100% | 100% |
| 2 | 49 ± 9% | 32 ± 6% |
| 3 | 82 ± 7% | 71 ± 7% |
| 4 | 64 ± 4% | 56 ± 7% |
| 5 | 59 ± 5% | 44 ± 3% |
| 6 | 78 ± 7% | 65 ± 3% |
| 7 | 94 ± 10% | 89 ± 8% |
| 8 | 92 ± 4% | 87 ± 6% |
| 9 | 92 ± 7% | 94 ± 3% |

Data from Table 2 clearly show that intracranial bacterial DNA injection led to a significant progression of neurodegeneration in MitoPark mice, while the injection with human DNA led to a significantly milder progression of the disease. Surprisingly, nervous system-specific transgenic expression of DNase I led to the amelioration of Parkinson's like disease development triggered with either bacterial or human DNA. Under the same conditions, liver-specific expression of DNase I or systemic DNase I protein administration led to a significantly lower efficacy against Parkinson's like disease development triggered by bacterial DNA. Both liver-specific expression of DNase I and systemic DNase I protein administration were similarly effective to nervous system-specific transgenic expression of DNase I in the cases where the disease was triggered by administration of human DNA.

### Example 4

### Effects of Different Ways of DNase I Delivery on Amyotrophic Lateral Sclerosis (ALS) Triggered by DNA

The therapeutic effect of different ways of DNase I delivery was studied in SOD1-G93A mice (ALS SOD1 G93A (strain: B6SJL-Tg(SOD1-G93A)1Gur/J)) mice. ALS was facilitated by intracranial injection with P. gingivalis strain 1731 DNA (5 μL 1 μg) or human DNA (5 μL 1 μg) into the mice at 4 weeks of age. DNA was isolated as previously described.

Anc80L65-DNAse I hyperactive vector with a microglia-specific promoter (Anc80L65-F4/80 promoter-hDNaseI (hyperactive)correct leader-WPRE.bGH) was injected in CSF, and the ApoEHCR enhancer-hAAT promoter-hDNaseI (hyperactive)correct leader-WPRE Xinact vector with a liver-specific promoter was iv injected 7 days after the DNA injection. Treatment was initiated 1 week after the DNA injection (week 5) and lasted for 12 weeks.

The ALS progression was assessed with motor unit number estimation (MUNE) and electrical impedance myography (EINI) measurements. MUNE measurement was performed with the TECA Synergy T2 EMG Monitor System (Viasys, Madison, Wis.) on the right hind limb as described previously (Shefner et al. (2006) Muscle Nerve 34:603-607). Briefly, a supramaximal compound motor action potential was measured; then, the stimulus intensity was suggested as zero and gradually increased until the identification of the first motor unit potential. Stimulus intensity was gradually increased until a total of 20 such steps in amplitude were determined. For the EINI measurement, the animals were placed under anesthesia. The fur on the study zone was removed for the electrode placement over the gastrocnemius muscle (Li et al. (2012) PloS ONE 7:e45004). HIM measurements were performed with the Imp SFB7® bioimpedance spectroscopy device (Impedimed, US) with a 50 kHz frequency.

Animals were allocated to the following experimental groups and received the treatments accordingly.
Group 1: SOD1 G93A mice intraspinally injected with sterile saline.
Group 2: SOD1 G93A mice intracerebrally injected with E. coli DNA 5 μL 1 ng+intraspinally injected with sterile saline.
Group 3: SOD1 G93A mice intracerebrally injected with E. coli DNA 5 μL 1 ng+intraspinally injected with nervous system-specific Anc80L65-DNAse I hyperactive (Anc80L65-CMV-F4/80-hDNaseI (hyperactive)correct leader-WPRE.bGH).
Group 4: SOD1 G93A mice intracerebrally injected with E. coli DNA 5 μL 1 ng+iv injected ApoEHCR enhancer-hAAT promoter-hDNaseI (hyperactive)correct leader-WPRE Xinact (liver specific).
Group 5: SOD1 G93A mice intracerebrally injected with E. coli DNA 5 μL 1 ng+ip injected with DNase I protein (Genentech) 30000 Kunitz Units/kg/day 2 times a day.
Group 6: SOD1 G93A mice intracerebrally injected with human DNA 5 μL 1 ng+intraspinally injected with sterile saline.
Group 7: SOD1 G93A mice intracerebrally injected with human DNA 5 μL 1 ng+intraspinally injected with nervous system-specific Anc80L65-DNAse I hyperactive (Anc80L65-CMV-F4/80-hDNaseI (hyperactive)correct leader-WPRE.bGH).
Group 8: SOD1 G93A mice intracerebrally injected with human DNA 5 μL 1 ng+iv injected ApoEHCR enhancer-hAAT promoter-hDNaseI (hyperactive)correct leader-WPRE Xinact (liver specific).
Group 9: SOD1 G93A mice intracerebrally injected with human DNA 5 μL 1 ng+ip injected with DNase I 30000 Kunitz Units/kg/day 2 times a day 7 day a week.

The results are summarized in Table 3.

TABLE 3
MUNE and EIM measurements

| | Paw grip endurance (sec) | | MUNE (Motor unit) | | Phase (Degree) | |
|---|---|---|---|---|---|---|
| Group | 6 wk | 12 wk | 6 wk | 12 wk | 6 wk | 12 wk |
| Group 1 | 85 | 74 | 78 | 61 | 18 | 13 |
| Group 2 | | 32 | | 44 | | 8 |
| Group 3 | | 84 | | 75 | | 17 |
| Group 4 | | 62 | | 56 | | 10 |
| Group 5 | | 59 | | 53 | | 9 |
| Group 6 | | 59 | | 76 | | 12 |
| Group 7 | | 84 | | 72 | | 16 |
| Group 8 | | 91 | | 75 | | 18 |
| Group 9 | | 81 | | 74 | | 17 |

Data of Table 3 clearly show that bacterial DNA injection led to a significant progression of ALS in mice, while the injection with human DNA led to a substantially milder progression of the disease. Surprisingly, nervous system-specific transgenic expression of DNase I led to the amelioration of development of ALS triggered by either bacterial or human DNA. Liver-specific expression of DNase I or systemic DNase I protein administration led to a significantly lower efficacy against ALS triggered by bacterial DNA but to a similar efficacy against ALS triggered by human DNA.

### Example 5

### Effects of AAV5-DNase I Vector with Microglia-Specific Promoter on ALS Animal Model

The therapeutic effect of single intraspinal injection of microglia-specific AAV5-DNase I wild type vector (AAV5-TMEM119 promoter-hDNaseI (wild type)) was studied in SOD1-G3A mice (ALS SOD1 G93A (strain: B6SJL-Tg(SOD1-G93A)1Gur/J) mice, obtained from Jackson Laboratories) with an increased blood brain barrier (BBB) permeability. Increased BBB permeability was induced by IV injection of 0.01 mmol/kg E-cadherin Peptide (Abbiotec). ALS was facilitated by intravenous injection with Bacillus intestinalis strain 1731 DNA (10 μL 1000 ng) into the mice at 4 weeks of age. DNA was isolated as previously described. Treatment was initiated 1 week after the DNA injection (week 5).

The ALS progression was assessed with motor unit number estimation (MUNE) and electrical impedance myography (EIM) measurements as described in Example 4.

Cell-specificity of the transduction with the developed vector was also studied. cfDNA and DNase I activity were measured in serum and CSF 2 days and 30 days after vector IV injection.

Circulating cfDNA was measured in plasma and CSF following the isolation of the DNA from 25 μl sample with QIAamp Circulating Nucleic Acid Kit (Qiagen) according to the manufacturer's protocol and was subsequently measured for absorbance at 260 nm. Data represent correlation for the untreated control group. Serum and CSF DNase I activity was measured using ORG590 (Orgentec) according to the manufacturer's protocol. Detection was performed using microplate photometer (Multiscan FC) at 450 nm with a correction wavelength of 620 nm. The impact of AAV5-DNase I was studied on Day 2 and Day 30 (Table 4).

Animals were allocated to the following experimental groups (n=8 per group) and received the treatments accordingly.
Group 1: Control—sterile saline IV injection
Group 2: Experimental—bacterial DNA IV injection
Group 3: Experimental—bacterial DNA IV injection+AAV5-DNase I (intraspinal) vector at 1.00×10⁸ genome copies (GC)/kg dose

The results of circulating cfDNA and DNase I activity are summarized in Table 4.

TABLE 4
cfDNA and DNase activity

| | cfDNA (%) | | | | DNase activity (mKuU/ml) | | | |
|---|---|---|---|---|---|---|---|---|
| | Blood | | CSF | | Blood | | CSF | |
| Group | 2 d | 30 d | 2 d | 30 d | 2 d | 30 d | 2 d | 30 d |
| Group 1 | 100 | 100 | 100 | 100 | 1.89 | 1.65 | 0.31 | 0.28 |
| Group 2 | 98 ± 8 | 102 ± 10 | 104 ± 3 | 95 ± 11 | 1.72 | 1.81 | 0.24 | 0.29 |
| Group 3 | 102 ± 6 | 99 ± 4 | 78 ± 6 | 68 ± 4 | 1.78 | 1.52 | 0.82 | 0.89 |

As shown in Table 4, the IV administration of the microglia-specific AAV5-DNase I vector led to the specific increase in DNase activity in the CSF, but not in the blood.

The impact of AAV5-DNase I on ALS progression is summarized in Table 5.

TABLE 5
MUNE and EIM measurements

| | Paw grip endurance (sec) | | | MUNE (Motor unit) | | | Phase (Degree) | | |
|---|---|---|---|---|---|---|---|---|---|
| Group | 6 wk | 12 wk | 18 wk | 6 wk | 12 wk | 18 wk | 6 wk | 12 wk | 18 wk |
| Group 1 | 85 | 74 | 34 | 78 | 61 | 45 | 18 | 13 | 11 |
| Group 2 | 85 | 32 | 9 | 78 | 44 | 19 | 18 | 10 | 8 |
| Group 3 | 85 | 84 | 76 | 78 | 75 | 71 | 18 | 17 | 16 |

As shown in Table 5, the injection of bacterial DNA triggered the progression of the ALS disease in the mouse model. Administration of AAV5-DNase I vector with the expression of DNase I under the control of a microglia-specific promoter significantly ameliorated the ALS progression.

### Example 6

### Effects of Different Ways of DNase I Delivery on Alzheimer's Disease Development Triggered by Oral Colonization with P. gingivalis or Intraspinal Injection with Human Herpesvirus (HHV)

To study the effect of (i) P. gingivalis oral infection or (ii) intraspinal human herpesvirus (HHV) infection on Amyloid 1-42 (Aβ1-42) and Tau levels in the brains, 40-week-old specific pathogen-free (SPF) female 3×TG mice were used. Mice were kept under a 12-hour light/dark cycle at 22° C. and 60° C. relative humidity with ad librum access to water.

For P. gingivalis infection, P. gingivalis solution (8_{log10}) was applied topically on the buccal surface of the alveolar ridge of the maxilla for 8 weeks every other day.

For HHV infection, animals were injected with 10e6 HEW-1 viral particles (VP)/mice once.

DNase I treatment was initiated 3 days before the initiation of the P. gingivalis or HHV-1 infection. Anc80L65-DNAse I hyperactive vector with a microglia-specific promoter (Anc80L65-Mef2 promoter-hDNaseI (hyperactive)correct leader-WPRE.bGH) was intraspinally injected, Anc80L65-DNAse I hyperactive vector with a neuron-specific promoter (Anc80L65-foxP2 promoter-hDNaseI (hyperactive)correct leader-WPRE.bGH) was intraventricularly injected, and Anc80L65-DNAse I hyperactive vector with a liver-specific promoter (Anc80L65-ApoEHCR enhancer-hAAT promoter-hDNaseI (hyperactive)correct leader-WPRE Xinact) was IV injected.

After 8 weeks, the mice were euthanized and brains were harvested and frozen. Amyloid deposition and tau phosphorylation were assessed via immunohistochemistry as previously described with some modifications from the CA1 region (Zenaro et al. (2015) Nat. Med. 21:880-886). The impact of different ways of DNase I delivery on tau hyperphosphorylation was studied using the AT8 mAb antibody specific for pSer202/Thr205 epitopes, which are among the earliest phosphorylated tau epitopes in patients with AD. Microglia activation was measured based on its density and shape.

Animals were allocated to the following experimental groups (n=5) and received the treatments accordingly.
Group 1: P. gingivalis administration
Group 2: P. gingivalis administration+Anc80L65-DNAse I hyperactive (Anc80L65-CMV-Mef2-hDNaseI (hyperactive)correct leader-WPRE.bGH)
Group 3: P. gingivalis administration+Anc80L65-DNAse I hyperactive (Anc80L65-CMV-foxP2-hDNaseI (hyperactive)correct leader-WPRE.bGH)
Group 4: P. gingivalis administration+ApoEHCR enhancer-hAAT promoter-hDNaseI (hyperactive)correct leader-WPRE Xinact
Group 5: P. gingivalis administration+iv DNase I protein 30000 Kunitz Units/kg/day 2 times a day
Group 6: HHV-1 administration
Group 7: HHV-1 administration+Anc80L65-DNAse I hyperactive (Anc80L65-CMV-Mef2-hDNaseI (hyperactive)correct leader-WPRE.bGH)
Group 8: HHV-1 administration+Anc80L65-DNAse I hyperactive (Anc80L65-CMV-foxP2-hDNaseI (hyperactive)correct leader-WPRE.bGH)
Group 9: HHV-1 administration+ApoEHCR enhancer-hAAT promoter-hDNaseI (hyperactive)correct leader-WPRE Xinact
Group 10: HHV-1 administration+DNase I protein (Genentech) 30000 Kunitz Units/kg/day 2 times a day.

The results are summarized in Table 6 below.

TABLE 6
Tau phosphorylation, amyloid
deposition and microglia activation

| | Tau | Amyloid | |
|---|---|---|---|
| | hyperphosphorylation | deposition | |
| | pixel²/2752 × 2208 | pixel²/2752 × 2208 | Microglia |
| Group | pixels | pixels | activation |
| 1 | 233,000 ± 34,000 | 75,000 ± 8,000 | 83,000 ± 9,000 |
| 2 | 129,000 ± 18,000 | 22,000 ± 9,000 | 21,000 ± 7,000 |
| 3 | 114,000 ± 13,000 | 20,000 ± 7,000 | 16,000 ± 8,000 |
| 4 | 182,000 ± 14,000 | 54,000 ± 3,000 | 53,000 ± 5,000 |
| 5 | 187,000 ± 19,000 | 49,000 ± 5,000 | 52,000 ± 7,000 |
| 6 | 287,000 ± 33,000 | 99,000 ± 7,000 | 88,000 ± 6,000 |
| 7 | 165,000 ± 21,000 | 34,000 ± 5,000 | 28,000 ± 4,000 |
| 8 | 159,000 ± 12,000 | 39,000 ± 6,000 | 22,000 ± 3,000 |
| 9 | 208,000 ± 14,000 | 68,000 ± 7,000 | 54,000 ± 5,000 |
| 10 | 224,000 ± 11,000 | 61,000 ± 4,000 | 57,000 ± 4,000 |

Data from Table 6 above clearly show that nervous system-specific transgenic expression of DNase I enzyme can significantly ameliorate Alzheimer's disease (AD) development and protein misfolding triggered by the colonization of oral microbiota with a particular microorganism or triggered with HHV-1 compared to the treatment with liver-specific vector encoding DNase I and systemic administration of a recombinant DNase I enzyme.

### Example 7

### Effects of Different Ways of DNase I Delivery on Alzheimer's Disease Development Triggered by DNA

The therapeutic effect of different ways of DNase I delivery was studied in 3×TG mice. AD development was facilitated by intraspinal injection with E. coli DNA (5 μL 1 μg) or human DNA (5 μL 1 μg) into 36-week-old specific pathogen-free female 3×TG mice.

Anc80L65-DNAse I hyperactive vector with a microglia-specific promoter (Anc80L65-CMV-Mef2-hDNaseI (hyperactive)correct leader-WPRE.bGH) was intraspinally injected and Anc80L65-DNAse I hyperactive vector with a liver-specific promoter (ApoEHCR enhancer-hAAT promoter-hDNaseI (hyperactive)correct leader-WPRE Xinact) was IV injected. DNase I treatment was initiated 4 weeks after DNA administration.

After 8 weeks, the mice were euthanized, brains were harvested and frozen.

Amyloid deposition and tau phosphorylation were assessed as described in Example 6. The impact of different ways of DNase I delivery on tau hyperphosphorylation was studied using the AT8 mAb antibody specific for pSer202/Thr205 epitopes. Microglia activation was measured based on its density and shape.

Animals were allocated to the following experimental groups (n=5) and received the treatments accordingly.
Group 1: Control 3×TG mice intraspinally injected with E. coli DNA 5 μL 1 ng
Group 2: 3×TG mice intraspinally injected with E. coli DNA 5 μL 1 ng+Anc80L65-DNAse I hyperactive (Anc80L65-CMV-Mef2-hDNaseI (hyperactive)correct leader-WPRE.bGH)
Group 3: 3×TG mice intraspinally injected with E. coli DNA 5 μL 1 ng+ApoEHCR enhancer-hAAT promoter-hDNaseI (hyperactive)correct leader-WPRE Xinact
Group 4: 3×TG mice intraspinally injected with E. coli DNA 5 μL 1 ng+DNase I 30000 Kunitz Units/kg/day 2 times a day
Group 5: 3×TG mice intraspinally injected with Human DNA 5 μL 1 ng
Group 6: 3×TG mice intraspinally injected with Human DNA 5 μL 1 ng+Anc80L65-DNAse I hyperactive (Anc80L65-CMV-Mef2-hDNaseI (hyperactive)correct leader-WPRE.bGH)
Group 7: 3×TG mice intraspinally injected with Human DNA 5 μL 1 ng+ApoEHCR enhancer-hAAT promoter-hDNaseI (hyperactive)correct leader-WPRE Xinact
Group 8: 3×TG mice intraspinally injected with Human DNA 5 μL 1 ng+DNase I protein (Genentech) 30000 Kunitz Units/kg/day 2 times a day

The results are summarized in Table 7 below.

TABLE 7
Tau phosphorylation and microglia activation

| | Tau | |
|---|---|---|
| | hyperphosphorylation | |
| | pixel²/2752 × 2208 | Microglia |
| Group | pixels | activation |
| 1 | 354,000 ± 56,000 | 101,000 ± 7,000 |
| 2 | 194,000 ± 21,000 | 42,000 ± 3,000 |
| 3 | 225,000 ± 20,000 | 72,000 ± 4,000 |
| 4 | 254,000 ± 11,000 | 76,000 ± 5,000 |
| 5 | 164,000 ± 115,000 | 66,000 ± 8,000 |
| 6 | 117,000 ± 10,000 | 28,000 ± 4,000 |
| 7 | 108,000 ± 11,000 | 32,000 ± 2,000 |
| 8 | 117,000 ± 5,000 | 33,000 ± 3,000 |

Data from Table 7 clearly show that bacterial DNA injection led to a significant progression of Alzheimer's disease (AD) in mice, while the injection with human DNA led to a significantly milder progression of the disease. nervous system-specific transgenic expression of DNase I led to the amelioration of AD development, when triggered with either bacterial or human DNA. Liver-specific expression of DNase I and systemic DNase I protein administration led to a significantly lower efficacy against AD triggered by bacterial DNA. However, both liver-specific expression of DNase I and systemic DNase I protein administration had a similar effect to nervous system-specific transgenic expression of DNase I in cases where the disease was triggered by the administration of human DNA.

### Example 8

### Effects of Lentivirus-Mediated Transgenic Delivery of DNase I on the Development of Alzheimer's Disease in an Animal Model

The DNA molecule coding for the hyperactive actin-resistant DNase I mutant (SEQ ID NO: 5) with DNase I secretory signal sequence replaced by the IL2 secretory signal sequence was synthesized (GeneCust) and cloned into the pLV2 lentiviral vector (Clontech) under the control of the neuron-specific CMV promoter (CMV promoter-pLV2-IL2ss-DNAseI mut). The 293T lentiviral packaging cell line (Clontech) was cultured in DMEM (Gibco).

To study the effect of P. gingivalis oral infection on A(31-42 and Tau levels in the brains, 40-week-old specific pathogen-free (SPF) female BALB/c mice were purchased from Jackson laboratories. Mice were kept under a 12-hour light/dark cycle at 22° C. and 60% relative humidity with ad librum access to water.

For infection, P. gingivalis solution (8_{log10}) was applied topically on the buccal surface of the alveolar ridge of the maxilla for 8 weeks every other day. DNase I vector was intraspinally injected (prophylactic and prevention regimen) 3 days before the initiation of the P. gingivalis infection. After 8 weeks, the mice were euthanized, brains were harvested and frozen.

Amyloid deposition and tau phosphorylation were assessed via immunohistochemistry as described in Example 6. The impact of DNase I transgene on tau hyperphosphorylation was studied using the AT8 mAb antibody specific for pSer202/Thr205 epitopes. Microglia activation was measured based on its density and shape.

Animals were allocated to the following experimental groups (n=8) and received the treatments accordingly.
Group 1: Experimental—P. gingivalis administration
Group 2: Experimental—P. gingivalis administration +CMV-pLV2-IL2ss-DNAseI mut (intraspinal) vector at 1.00×10¹¹ GC/kg dose

The results are summarized in Table 8.

TABLE 8
Tau hyperphosphorylation, amyloid
deposition and microglia activation

| | Tau | Amyloid | |
|---|---|---|---|
| | hyperphosphorylation | deposition | |
| | pixel²/2752 × 2208 | pixel²/2752 × 2208 | Microglia |
| Group | pixels | pixels | activation |
| 1 | 233,000 ± 34,000 | 75,000 ± 8,000 | 83,000 ± 9,000 |
| 2 | 129,000 ± 28,000 | 42,000 ± 9,000 | 51,000 ± 7,000 |

Data from the Table 8 above clearly show that nervous system-specific transgenic expression of DNase I enzyme using a lentivirus vector can significantly ameliorate Alzheimer's Disease (AD) development and protein misfolding triggered by the colonization of oral microbiota with a particular microorganism.

### Example 9

### Difference of the Efficacy of Transgenic Delivery of DNase I with Nervous System-Specific and Liver-Specific Promoters on the Alzheimer's Disease Development in an Animal Model

The therapeutic effect of single injections of two gene therapy Anc80L65 vectors were studied: one with the liver specific promoter, Anc80L65-ApoEHCR enhancer-hAAT promoter-hDNaseI (hyperactive)correct leader-WPRE Xinact; and another with nervous system-specific promoter, Anc80L65-CMV promoter-hDNaseI (hyperactive)correct leader-WPRE.bGH.

The triple transgenic (APPsw/PS1M146V/TauP301L, 3×Tg-AD) 9-month-old mice were used. Animals received a single vector injection and were monitored for 3 months. Alzheimer's disease progression was enhanced by intracerebral injection of E. coli DNA 5 μL 1 ng (isolated as discussed above) in the corresponding animals. Animals were anesthetized by intraperitoneal injection of ketamine/xylazine. Amyloid 1-42 (Aβ1-42) and phosphorylated tau (p-tau Thr181) in the brain tissues were measured by enzyme linked immuno-sorbent assay (ELISA) with the Cell Like technology kit (D9F4G, Life Technologies, US), according to the manufacturer's instructions.

Animals were allocated to the following experimental groups and received the treatments accordingly.
Group 1: 3×Tg-AD mice intracerebrally injected with sterile saline+intraspinally injected with sterile saline.
Group 2: 3×Tg-AD mice intracerebrally injected with sterile saline+IV injected with Anc80L65 ApoEHCR enhancer-hAAT promoter-hDNaseI (hyperactive)correct leader-WPRE Xinact at 1.00×10¹¹ GC/kg dose.
Group 3: 3×Tg-AD mice intracerebrally injected with sterile saline+intraspinally injected with Anc80L65 CMV promoter-hDNaseI (hyperactive)correct leader-WPRE.bGH at 1.00×10¹¹ GC/kg dose.
Group 4: 3×Tg-AD mice intracerebrally injected with sterile saline+intracerebrally injected with Anc80L65 CMV promoter-hDNaseI (hyperactive)correct leader-WPRE.bGH at 1.00×10¹¹ GC/kg dose.
Group 5: 3×Tg-AD mice intracerebrally injected with E. coli DNA 5 μL 1 ng+intraspinally injected with sterile saline.
Group 6: 3×Tg-AD mice intracerebrally injected with E. coli DNA 5 μL 1 ng+IV injected with Anc80L65 ApoEHCR enhancer-hAAT promoter-hDNaseI (hyperactive)correct leader-WPRE Xinact at 1.00×10¹¹ GC/kg dose.
Group 7: 3×Tg-AD mice intracerebrally injected with E. coli DNA 5 μL 1 ng saline+intraspinally injected with Anc80L65 CMV promoter-hDNaseI (hyperactive)correct leader-WPRE.bGH at 1.00×10¹¹ GC/kg dose.
Group 8: 3×Tg-AD mice intracerebrally injected with E. coli DNA 5 μL 1 ng+intracerebrally injected with Anc80L65 CMV promoter-hDNaseI (hyperactive)correct leader-WPRE.bGH at 1.00×10¹¹ GC/kg dose.

The results of Aβ1-42 and p-tau Thr181 ELISAs are summarized in Tables 9 and 10 below. Data show mean+/−standard deviation (SD).

TABLE 9
Effect of the transgenic expression of
DNase I on Aβ1-42 in the prefrontal
cortex the brain of 3xTg-AD mice

| Group | Aβ 1-42 (pg/mL) |
|---|---|
| 1 | 174 ± 22 |
| 2 | 87 ± 10 |
| 3 | 76 ± 6 |
| 4 | 73 ± 11 |
| 5 | 224 ± 13 |
| 6 | 122 ± 16 |
| 7 | 74 ± 9 |
| 8 | 72 ± 11 |

TABLE 10
Effect of the transgenic expression of
DNase I on tau phosphorylation 1 cortex
the entorhinal cortex of 3xTg-AD mice

| | Phosphorilated tau Thr 181/total |
|---|---|
| Group | protein (pg/μg) (in thousands, 000) |
| 1 | 98 ± 11 |
| 2 | 34 ± 9 |
| 3 | 37 ± 8 |
| 4 | 31 ± 7 |
| 5 | 139 ± 19 |
| 6 | 56 ± 10 |
| 7 | 34 ± 8 |
| 8 | 38 ± 10 |

Data from Tables 9 and 10 clearly show that intracranial injection of E. coli DNA promotes β-amyloid deposition and tau phosphorylation (Group 1 vs Group 5, p<0.05). The use of liver-specific vector is effective against Alzheimer's Disease (AD) progression in all cases; however, vectors with nervous system-specific promoters are unexpectedly even more effective, particularly in the cases where AD progression is stimulated by the protein misfolding targeted with E. coli DNA.

### Example 10

### Effects of Transgenic Delivery of DNase I on the Brain Cancer

The therapeutic effect of single injections of microglia-specific AAV9-DNAse I hyperactive (AAV9-F4/80 promoter-hDNaseI (hyperactive)correct leader-WPRE.bGH) was studied in a brain glioma model.

For the intracranial glioma xenograft model male 6 to 8-week-old NOD-SCID mice were used. Animals were anesthetized by intraperitoneal injection of ketamine/xylazine. The heads were shaved, sterilized and animals were fixed. A burr hole was drilled into the skull, following a 7 mm skin incision along the sagittal suture line. 2×10⁵ malignant human brain glioma cells (GBM8401) in 1.5 μL culture medium were then injected into the brain (left hemisphere). The burr holes in the skull were sealed with bone wax and the wound was covered with 3M Tegaderm Alginate AG Silver Dressing.

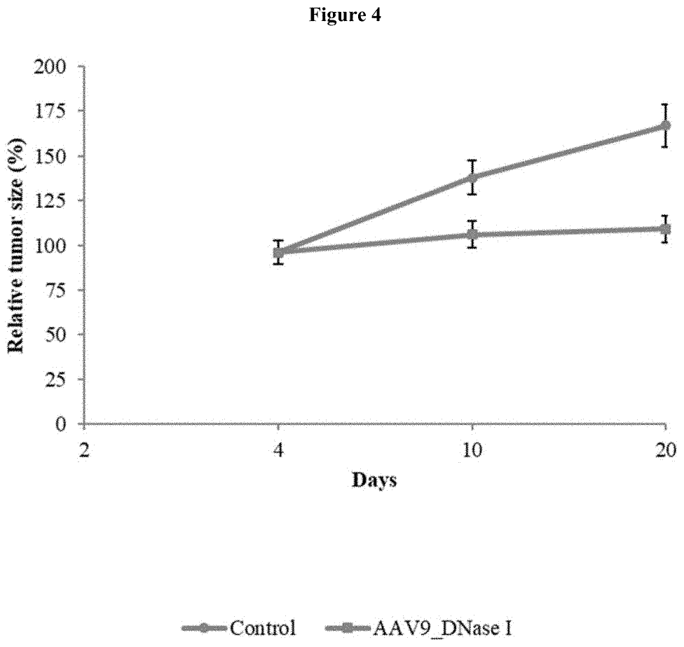

Therapy was assessed from the day 4 after tumor implantation. Tumor growth was assessed within the next 14 days. Data reflect an increase in tumor size (see FIG. 4) which was based on data obtained from bioluminescence images.

Animals were allocated to the following experimental groups (n=15 per group) and received the treatments accordingly.
Group 1: Control—no treatment
Group 2: Experimental—intraspinally injected with AAV9-DNAse I vector (AAV9-F4/80-hDNaseI (hyperactive)correct leader-WPRE.bGH) at 1.00×10⁹ GC/kg dose

The results (see FIG. 4) show that transgenic expression of the DNase I vector with a nervous system-specific promoter significantly inhibited brain tumor growth.

### REFERENCES

Song, L. et al., “NLRP3 Inflammasome in Neurological Diseases, from Functions to Therapies” Front. Cell. Neurosci., 2017, Vol. 11, No. 63.
Fleischhacker M. et al., “Circulating nucleic acids (CNAs) and cancer: a survey” Biochim Biophys Acta, 2007, 1775(1): 181-232.
Demers, M. at al., “Cancers predispose neutrophils to release extracellular DNA traps that contribute to cancer-associated thrombosis” PNAS, 2012, 109(32):13076-13081.
Garcia-Olmo D. C. and Garcia-Olmo, D. “Biological role of cell-free nucleic acids in cancer: the theory of genometastasis” Crit Rev Oncolog., 2013, 18: 153-161.
Sawyers 2008 C. L., “The cancer biomarker problem” Nature, 2008, 452(7187):548-552.
Butt A. N. et al. Overview of circulating nucleic acids in plasma/serum. Ann. N.Y. Acad. Sci., 2008, 1137:236-242.
Schwarzenbach H. et al., “Detection and monitoring of cell-free DNA in blood of patients with colorectal cancer” Ann. N.Y. Acad. Sci., 2008, 1137:190-196.
Zenaro E, et al., Neutrophils promote Alzheimer's disease-like pathology and cognitive decline via LFA-1 integrin. Nat Rev Nephrol., 2015, Author manuscript; available in PMC 2017 Jul. 14.
Fushi, W. et al., “Extracellular DNA in Pancreatic Cancer Promotes Cell Invasion and Metastasis” Cancer Res., 2013, 73:4256-4266.
Tohme, S. et al., “Neutrophil Extracellular Traps Promote the Development and Progression of Liver Metastases after Surgical Stress” Cancer Res., 2016 Mar. 15, 76(6): 1367-1380.
Patutina, O. et al., Inhibition of metastasis development by daily administration of ultralow doses of RNase A and DNase I. Biochimie., 2011 April, 93(4): 689-96.
Dan Li, DNase I Treatment Reduces GVHD in Mice. Biology of Blood and Marrow Transplantation, Volume 21, Issue 2, Page 5339.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A recombinant adeno-associated virus (rAAV) expression vector comprising (i) a capsid protein and (ii) a nucleic acid comprising a promoter operably linked to a nucleotide sequence encoding an enzyme which has a deoxyribonuclease (DNase) activity, wherein the promoter is a nervous system-specific promoter.

2. The vector of claim 1, wherein the AAV is selected from serotype 1 (AAV1), AAV2, AAV3, AAV4, AAV5, AAV6, AAV7, AAV5, AAV9, AAV10, AAV11, AAV12, AAVrh10, AAVLK03, AAVLK06, AAVLK12, AAV-KP1, AAV-F, AAVDJ, AAV-PHP.B, AAVhu37, AAVrh64R1, and Anc 80.

3. The vector of claim 2, wherein the AAV is from serotype 5 (AAV5).

4. The vector of claim 2, wherein the AAV is from serotype 9 (AAV9).

5. The vector of claim 2, wherein the AAV is from serotype Anc 80.

6. The vector of any one of claims 1-5, wherein the capsid protein comprises one or more mutations which improve efficiency and/or specificity of the delivery of the vector to the nervous system as compared to the corresponding wild-type capsid protein.

7. The vector of claim 6, wherein the improved efficiency and/or specificity of the delivery of the vector to the nervous system results in a substantially increased expression of the enzyme in the nervous system as compared to other tissues and organs.

8. The vector of claim 6 or claim 7, wherein the one or more mutations in the capsid protein are selected from the group consisting of S279A, S671A, K137R, T252A, and any combinations thereof.

9. The vector of claim 1, wherein the capsid protein comprises the sequence SEQ ID NO:34.

10. The vector of claim 9, wherein the capsid protein consists of the sequence SEQ ID NO:34.

11. The vector of claim 1, wherein the capsid protein comprises the sequence SEQ ID NO: 35.

12. The vector of claim 11, wherein the capsid protein consists of the sequence SEQ ID NO: 35.

13. The vector of any one of claims 1-12, wherein the nucleic acid further comprises two AAV inverted terminal repeats (ITRs), wherein the ITRs flank the nucleotide sequence encoding the enzyme which has a DNase activity.

14. A recombinant retroviral vector comprising (i) a capsid protein and (ii) a nucleic acid comprising a promoter operably linked to a nucleotide sequence encoding an enzyme which has a deoxyribonuclease (DNase) activity, wherein the promoter is a nervous system-specific promoter.

15. The vector of claim 14 which is a lentiviral vector.

16. The vector of any one of claims 1-15, wherein the nervous system-specific promoter mediates a substantially increased expression of the enzyme in the nervous system as compared to other tissues and organs.

17. The vector of any one of claims 1-16, wherein the nervous system-specific promoter is a microglia-specific promoter.

18. The vector of any one of claims 1-16, wherein the nervous system-specific promoter is a neuron-specific promoter.

19. The vector of any one of claims 1-18, wherein the nervous system-specific promoter is selected from the group consisting of F4/80 promoter, CD68 promoter, TMEM119 promoter, CX3CR1 promoter, CMV promoter, MEF2 promoter, FoxP2 promoter, Iba1 promoter, TTR promoter, CD11b promoter, c-fes promoter, NSE promoter, synapsin promoter, CamKII promoter, α-CaMKII promoter, VGLUT1 promoter, glial fibrillary acidic protein (GFAP) promoter, and GfaABC1D-dYFP promoter.

20. The vector of claim 19, wherein the nervous system-specific promoter is a F4/80 promoter.

21. The vector of claim 20, wherein the F4/80 promoter comprises the sequence SEQ ID NO: 38.

22. The vector of claim 21, wherein the F4/80 promoter consists of the sequence SEQ ID NO: 38.

23. The vector of claim 19, wherein the nervous system-specific promoter is a CMV promoter.

24. The vector of claim 23, wherein the CMV promoter comprises the sequence SEQ ID NO: 37.

25. The vector of claim 24, wherein the CMV promoter consists of the sequence SEQ ID NO: 37.

26. The vector of claim 19, wherein the nervous system-specific promoter is a TMEM119 promoter.

27. The vector of claim 26, wherein the TMEM119 promoter comprises the sequence SEQ ID NO: 39.

28. The vector of claim 27, wherein the TMEM119 promoter consists of the sequence SEQ ID NO: 39.

29. The vector of claim 19, wherein the nervous system-specific promoter is a MEF2 promoter.

30. The vector of claim 29, wherein the MEF2 promoter comprises the sequence SEQ ID NO: 40.

31. The vector of claim 30, wherein the MEF2 promoter consists of the sequence SEQ ID NO: 40.

32. The vector of claim 19, wherein the nervous system-specific promoter is a FoxP2 promoter.

33. The vector of claim 23, wherein the FoxP2 promoter comprises the sequence SEQ ID NO: 41.

34. The vector of claim 24, wherein the FoxP2 promoter consists of the sequence SEQ ID NO: 41.

35. The vector of claim 19, wherein the nervous system-specific promoter is a synapsin promoter.

36. The vector of claim 35, wherein the synapsin promoter comprises the sequence SEQ ID NO: 36.

37. The vector of claim 36, wherein the synapsin promoter consists of the sequence SEQ ID NO: 36.

38. The vector of any one of claims 1-37, wherein the enzyme which has a DNase activity is selected from the group consisting of DNase I, DNase X, DNase y, DNase1L1, DNase1L2, DNase 1L3, DNase II, DNase IIa, DNase Caspase-activated DNase (CAD), Endonuclease G (ENDOG), Granzyme B (GZMB), phosphodiesterase I, lactoferrin, acetylcholinesterase, and mutants or derivatives thereof.

39. The vector of claim 38, wherein the enzyme which has a DNase activity is DNase I or a mutant or derivative thereof.

40. The vector of claim 39, wherein the DNase I is a human DNase I or a mutant or derivative thereof.

41. The vector of claim 39 or claim 40, wherein the DNase I mutant comprises one or more mutations in an actin binding site.

42. The vector of claim 41, wherein the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof.

43. The vector of claim 42, wherein one of the mutations in the actin-binding site is a mutation at Ala-114.

44. The vector of any one of claims 39-43, wherein the DNase I mutant comprises one or more mutations increasing DNase activity.

45. The vector of claim 44, wherein said one or more mutations increasing DNase activity are selected from the group consisting of Q9R, E13R, E13K, T14R, T14K, H44R, H44K, N74K, A114F, and any combinations thereof.

46. The vector of claim 45, wherein said one or more mutations increasing DNase activity are selected from the group consisting of Q9R, E13R, N74K and A114F, and any combinations thereof.

47. The vector of claim 46, wherein the DNase I mutant comprises the mutations Q9R, E13R, N74K, and A114F.

48. The vector of any one of claims 39-47, wherein the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, Al 14C, Al 14R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P7OS, S68N:P70T, S94N:Y96S, S94N:Y96T, and any combinations thereof.

49. The vector of claim 39 or claim 40, wherein the DNase I mutant comprises a sequence having at least 80% sequence identity to the sequence of SEQ ID NO: 5.

50. The vector of claim 49, wherein the DNase I mutant comprises the sequence SEQ ID NO: 5.

51. The vector of claim 50, wherein the DNase I mutant consists of the sequence SEQ ID NO: 5.

52. The vector of claim 50 or claim 51, wherein the nucleic acid comprises a nucleotide sequence which is at least 85% identical to SEQ ID NO: 21.

53. The vector of claim 52, wherein the nucleic acid comprises the nucleotide sequence SEQ ID NO: 21.

54. The vector of claim 39 or claim 40, wherein the DNase I mutant comprises a sequence having at least 80% sequence identity to the sequence of SEQ ID NO: 2.

55. The vector of claim 54, wherein the DNase I mutant comprises the sequence SEQ ID NO: 2.

56. The vector of claim 55, wherein the DNase I mutant consists of the sequence SEQ ID NO: 2.

57. The vector of claim 55 or claim 56, wherein the nucleic acid comprises a nucleotide sequence which is at least 85% identical to SEQ ID NO: 19.

58. The vector of claim 57, wherein the nucleic acid comprises the nucleotide sequence SEQ ID NO: 19.

59. The vector of claim 39 or claim 40, wherein the DNase I comprises the sequence SEQ ID NO: 4.

60. The vector of claim 59, wherein the DNase I consists of the sequence SEQ ID NO: 4.

61. The vector of claim 59 or claim 60, wherein the nucleic acid comprises a nucleotide sequence which is at least 85% identical to SEQ ID NO: 23.

62. The vector of claim 61, wherein the nucleic acid comprises the nucleotide sequence SEQ ID NO: 23.

63. The vector of claim 39 or claim 40, wherein the DNase I comprises the sequence SEQ ID NO: 1.

64. The vector of claim 63, wherein the DNase I consists of the sequence SEQ ID NO: 1.

65. The vector of claim 63 or claim 64, wherein the nucleic acid comprises a nucleotide sequence which is at least 85% identical to SEQ ID NO: 22.

66. The vector of claim 65, wherein the nucleic acid comprises the nucleotide sequence SEQ ID NO: 22.

67. The vector of claim 65 or claim 66, wherein the nucleic acid comprises a nucleotide sequence which is at least 85% identical to SEQ ID NO: 32.

68. The vector of claim 67, wherein the nucleic acid comprises the nucleotide sequence SEQ ID NO: 32.

69. The vector of any one of claims 1-68, wherein the enzyme which has a DNase activity is a fusion protein comprising (i) a DNase enzyme or a fragment thereof linked to (ii) an albumin or an Fc or a fragment thereof.

70. The vector of any one of claims 1-69, wherein the sequence encoding the enzyme which has a DNase activity comprises a sequence encoding a secretory signal sequence, wherein said secretory signal sequence mediates effective secretion of the enzyme.

71. The vector of claim 70, wherein the secretory signal sequence is selected from the group consisting of DNase I secretory signal sequence, IL2 secretory signal sequence, the albumin secretory signal sequence, the P-glucuronidase secretory signal sequence, the alkaline protease secretory signal sequence, and the fibronectin secretory signal sequence.

72. The vector of claim 70, wherein the secretory signal sequence comprises the sequence MRGMKLLGALLALAALLQGAVS (SEQ ID NO: 6) or MYRMQLLSCIALSLALVTNS (SEQ ID NO: 7).

73. The vector of claim 72, wherein the secretory signal sequence consists of the sequence MRGMKLLGALLALAALLQGAVS (SEQ ID NO: 6) or MYRMQLLSCIALSLALVTNS (SEQ ID NO: 7).

74. The vector of claim 70, wherein the secretory signal sequence comprises a sequence having at least 80% sequence identity to the sequence of MRGMKLLGALLALAALLQGAVS (SEQ ID NO: 6) or a sequence having at least 85% sequence identity to the sequence of MYRMQLLSCIALSLALVTNS (SEQ ID NO: 7).

75. The vector of claim 74, wherein the sequence encoding the secretory signal sequence comprises a nucleotide sequence which is at least 85% identical to SEQ ID NO: 20.

76. The vector of claim 75, wherein the sequence encoding the secretory signal sequence comprises the nucleotide sequence SEQ ID NO: 20.

77. The vector of claim 70, wherein the secretory signal sequence comprises the sequence MRYTGLMGTLLTLVNLLQLAGT (SEQ ID NO: 25).

78. The vector of claim 77, wherein the secretory signal sequence consists of the sequence MRYTGLMGTLLTLVNLLQLAGT (SEQ ID NO: 25).

79. The vector of claim 70, wherein the secretory signal sequence comprises a sequence having at least 80% sequence identity to the sequence of MRYTGLMGTLLTLVNLLQLAGT (SEQ ID NO: 25).

80. The vector of claim 79, wherein the sequence encoding the secretory signal sequence comprises a nucleotide sequence which is at least 85% identical to SEQ ID NO: 27.

81. The vector of claim 80, wherein the sequence encoding the secretory signal sequence comprises the nucleotide sequence SEQ ID NO: 27.

82. The vector of any one of claims 1-81, wherein the nucleic acid further comprises one or more enhancers located upstream or downstream of the promoter.

83. The vector of claim 82, wherein the one or more enhancers are selected from the group consisting of nPE2 enhancer, Gal4 enhancer, foxP2 enhancer, Mef2 enhancer, and any combination thereof

84. The vector of any one of claims 1-83, wherein the nucleic acid further comprises a polyadenylation signal operably linked to the nucleotide sequence encoding the enzyme which has a DNase activity.

85. The vector of any one of claims 1-84, wherein the nucleic acid further comprises a Kozak sequence.

86. The vector of claim 85, wherein the Kozak sequence comprises the sequence 5′-GCCGCCACC-3′ (SEQ ID NO: 33).

87. The vector of any one of claims 1-86, wherein the nucleic acid further comprises a post-transcriptional regulatory element.

88. The vector of claim 87, wherein the post-transcriptional regulatory element is a woodchuck hepatitis post-transcriptional regulatory element (WPRE).

89. A pharmaceutical composition comprising the vector of any one of claims 1-88 and a pharmaceutically acceptable carrier and/or excipient.

90. A method for delivering an enzyme which has a deoxyribonuclease (DNase) activity to the nervous system in a subject in need thereof, comprising administering to the subject the vector of any one of claims 1-88 or the composition of claim 89.

91. A method for treating a disease associated with protein misfolding in a subject in need thereof, said method comprising administering to the subject a therapeutically effective amount of the vector of any one of claims 1-88 or the composition of claim 89.

92. The method of claim 91, wherein the protein misfolding is associated with the presence of cell-free DNA (cfDNA) in cerebrospinal fluid (CSF) or nervous system tissue(s).

93. The method of claim 92, wherein the cfDNA is microbial and/or viral cfDNA.

94. The method of claim 93, wherein the microbial cfDNA is bacterial cfDNA.

95. The method of claim 93, wherein the viral cfDNA is bacteriophage cfDNA.

96. The method of any one of claims 91-95, wherein the disease associated with protein misfolding is selected from neurodegenerative diseases, neurodevelopmental diseases, psychiatric diseases, autoimmune diseases, oncological diseases and infections.

97. The method of claim 96, wherein the disease associated with protein misfolding is a neurodegenerative disease selected from Alzheimer's disease, Parkinson's disease, Amyotrophic Lateral Sclerosis (ALS), multiple sclerosis, CADASIL Syndrome, Friedreich's ataxia, cerebellar ataxia, spinocerebellar ataxia, dementia (e.g., fronto-temporal dementia, frontotemporal dementia with parkinsonism-17 (FTDP-17), familial Danish dementia, and familial British dementia), prion-caused diseases, Lewy body diseases, an amyloidosis (e.g., hereditary cerebral hemorrhage with amyloidosis, senile systemic amyloidosis, amyloidosis with a hereditary cerebral hemorrhage, a primary systemic amyloidosis, a secondary systemic amyloidosis, a serum amyloidosis, senile systemic amyloidosis, a hemodialysis-related amyloidosis, a Finnish hereditary systemic amyloidosis, an Atrial amyloidosis, a Lysozyme systemic amyloidosis, an Insulin-related amyloidosis, or a Fibrinogen cf-chain amyloidosis), Spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm or dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia), Shy-Drager syndrome, spinal muscular atrophy, primary lateral sclerosis, hereditary spastic paraplegia, peroneal muscular atrophy (Charcot-Marie-Tooth), and hypertrophic interstitial polyneuropathy (Dejerine-Sottas). In one embodiment, the neurodegenerative disease is secondary to diabetes, rheumatoid arthritis, systemic lupus erythematosus (SLE), gout, metabolic syndrome, an amyloidosis, asthma, and prion disease.

98. The method of claim 96, wherein the disease associated with protein misfolding is a neurodevelopmental disease selected from autism, neural tube defects, attention deficit hyperactivity disorder, Dawn syndrome, cerebral palsy, Rett syndrome, Landau-Kleffner syndrome, Alexander disease, Alpers' disease, alternating hemiplegia, Angelman syndrome, ataxias and cerebellar or spinocerebellar degeneration, ataxia telangiectasia, attention deficit-hyperactivity disorder, autism spectrum disorders, Asperger syndrome, Batten disease, Canavan disease, Tourette syndrome, and impairments in vision and/or hearing.

99. The method of claim 96, wherein the disease associated with protein misfolding is a psychiatric disease selected from anxiety disorders, psychotic disorders, schizophrenia, bipolar disorder, depression, post-traumatic stress disorder, and epilepsy.

100. The method of claim 96, wherein the disease associated with protein misfolding is an autoimmune disease selected from autoimmune encephalitis, autoimmune-related epilepsy, central nervous system (CNS) vasculitis, Hashimoto's encephalopathy, steroid-responsive encephalopathy, neurosarcoidosis, Neuro-Behcet's disease, cerebral lupus, neuromyelitis optica, optic neuritis, diabetes, rheumatoid arthritis, systemic lupus erythematosus (SLE), gout, asthma, celiac disease, ankylosing spondylitis, vasculitis, pemphigus vulgaris,inflammatory bowel disease (IBD), Crohn's disease, and ulcerative colitis.

101. The method of claim 96, wherein the disease associated with protein misfolding is an oncological disease selected from astrocytoma, gliomas, glioblastoma, ependymoma, medulloblastoma, central nervous system (CNS) lymphoma, craniopharyngioma, glioblastoma, meningioma, pituitary carcinomas, pituitary adenomas, neurofibromatosis, embryonal tumors, tumors of the pineal region, tumors of meninges, choroid plexus tumors, neuronal and mixed neuronal-glial tumors, germ cell tumors, anaplastic astrocytoma, central neurocytoma, choroid plexus carcinoma, choroid plexus papilloma, dysembryoplastic neuroepithelial tumor, giant-cell glioblastoma, gliosarcoma, hemangiopericytoma, medulloepithelioma, neuroblastoma, neurocytoma, oligoastrocytoma, oligodendroglioma, optic nerve sheath meningioma, pilocytic astrocytoma, pinealoblastoma, pineocytoma, pleomorphic anaplastic neuroblastoma, pleomorphic xanthoastrocytoma, sphenoid wing meningioma, subependymal giant cell astrocytoma, subependymoma, trilateral retinoblastoma, and nervous system metastasis of any origin.

102. The method of claim 96, wherein the disease associated with protein misfolding is an infection selected from adenoviral infections, hepatitis B, hepatitis G, poxvirus infections, herpesvirus infections, papillomavirus infections, HIV infections, meningitis (bacterial, fungal, or viral), bacterial persistence in brain, fungal persistence in brain, pancreatitis, and peritonitis.

103. The method of any one of claims 91-95, wherein the disease associated with protein misfolding is selected from Alzheimer's disease, Parkinson's disease, Amyotrophic Lateral Sclerosis (ALS), multiple sclerosis, CADASIL Syndrome, stroke, Friedreich's ataxia, cerebellar ataxia, spinocerebellar ataxia, dementia, prion-caused diseases, Lewy body diseases, amyloidosis, spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski),corticodentatonigral degeneration, Hallervorden-Spatz disease, progressive familial myoclonic epilepsy, striatonigral degeneration, torsion dystonia, spasmodic torticollis and other dyskinesis, familial tremor, Gilles de la Tourette syndrome, cerebellar cortical degeneration, spinocerebellar degeneration, Shy-Drager syndrome, spinal muscular atrophy, primary lateral sclerosis, hereditary spastic paraplegia, peroneal muscular atrophy (Charcot-Marie-Tooth), hypertrophic interstitial polyneuropathy (Dejerine-Sottas), nervous system tumors, and secondary neurodegeneration.

104. The method of claim 103, wherein the nervous system tumors are selected from the group consisting of astrocytoma, gliomas, glioblastoma, ependymoma, medulloblastoma, CNS lymphoma, craniopharyngioma, glioblastoma, meningioma, pituitary carcinomas, pituitary adenoma, neurofibromatosis, embryonal tumors, tumors of the pineal region, tumors of meninges, choroid plexus tumors, neuronal and mixed neuronal-glial tumors, and germ cell tumors.

105. The method of claim 103, wherein the secondary neurodegeneration is selected from the group consisting of neurodegeneration resulting from destruction of neurons by neoplasm, edema, hemorrhage, stroke, trauma, immune attack, hypoxia, poisoning, metabolic defects, and infections.

106. A method for treating a neurodegenerative, neurodevelopmental, psychiatric, autoimmune or oncological disease or an infection associated with protein misfolding in a subject in need thereof, said method comprising administering to the subject a therapeutically effective amount of the vector of any one of claims 1-88 or the composition of claim 89.

107. The method of claim 106, wherein the neurodegenerative disease is Alzheimer's disease, Parkinson's disease, or Amyotrophic Lateral Sclerosis (ALS).

108. The method of claim 107, wherein the neurodegenerative disease is secondary to diabetes, rheumatoid arthritis, systemic lupus erythematosus (SLE), gout, metabolic syndrome, an amyloidosis, asthma, or prion disease.

109. The method of claim 106, wherein the neurodegenerative disease is selected from Alzheimer's disease, Parkinson's disease, Amyotrophic Lateral Sclerosis (ALS), multiple sclerosis, CADASIL Syndrome, Friedreich's ataxia, cerebellar ataxia, spinocerebellar ataxia, dementia (e.g., fronto-temporal dementia, frontotemporal dementia with parkinsonism-17 (FTDP-17), familial Danish dementia, and familial British dementia), prion-caused diseases, Lewy body diseases, an amyloidosis (e.g., hereditary cerebral hemorrhage with amyloidosis, senile systemic amyloidosis, an amyloidosis with a hereditary cerebral hemorrhage, a primary systemic amyloidosis, a secondary systemic amyloidosis, a serum amyloidosis, a senile systemic amyloidosis, a hemodialysis-related amyloidosis, a Finnish hereditary systemic amyloidosis, an Atrial amyloidosis, a Lysozyme systemic amyloidosis, an Insulin-related amyloidosis, or a Fibrinogen α-chain amyloidosis), Spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm or dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia), Shy-Drager syndrome, spinal muscular atrophy, primary lateral sclerosis, hereditary spastic paraplegia, peroneal muscular atrophy (Charcot-Marie-Tooth), and hypertrophic interstitial polyneuropathy (Dejerine-Sottas). In one embodiment, the neurodegenerative disease is secondary to diabetes, rheumatoid arthritis, systemic lupus erythematosus (SLE), gout, metabolic syndrome, an amyloidosis, asthma, and prion disease.

110. The method of claim 106, wherein the neurodevelopmental disease is selected from autism, neural tube defects, attention deficit hyperactivity disorder, Dawn syndrome, cerebral palsy, Rett syndrome, Landau-Kleffner syndrome, Alexander disease, Alpers' disease, alternating hemiplegia, Angelman syndrome, ataxias and cerebellar or spinocerebellar degeneration, ataxia telangiectasia, attention deficit-hyperactivity disorder, autism spectrum disorders, Asperger syndrome, Batten disease, Canavan disease, Tourette syndrome, and impairments in vision and/or hearing.

111. The method of claim 106, wherein the psychiatric disease is selected from anxiety disorders, psychotic disorders, schizophrenia, bipolar disorder, depression, post-traumatic stress disorder, and epilepsy.

112. The method of claim 106, wherein the autoimmune disease is selected from autoimmune encephalitis, autoimmune-related epilepsy, central nervous system (CNS) vasculitis, Hashimoto's encephalopathy, steroid-responsive encephalopathy, neurosarcoidosis, Neuro-Behcet's disease, cerebral lupus, neuromyelitis optica, optic neuritis, diabetes, rheumatoid arthritis, systemic lupus erythematosus (SLE), gout, asthma, celiac disease, ankylosing spondylitis, vasculitis, pemphigus vulgaris, inflammatory bowel disease (MD). Crohn's disease and ulcerative colitis.

113. The method of claim 106, wherein the oncological disease is selected from astrocytoma, gliomas, glioblastoma, ependymoma, medulloblastoma, central nervous system (CNS) lymphoma, craniopharyngioma, glioblastoma, meningioma, pituitary carcinomas, pituitary adenomas, neurofibromatosis, embryonal tumors, tumors of the pineal region, tumors of meninges, choroid plexus tumors, neuronal and mixed neuronal-glial tumors, germ cell tumors, anaplastic astrocytoma, central neurocytoma, choroid plexus carcinoma, choroid plexus papilloma, dysembryoplastic neuroepithelial tumor, giant-cell glioblastoma, gliosarcoma, hemangiopericytoma, medulloepithelioma, neuroblastoma, neurocytoma, oligoastrocytoma, oligodendroglioma, optic nerve sheath meningioma, pilocytic astrocytoma, pinealoblastoma, pineocytoma, pleomorphic anaplastic neuroblastoma, pleomorphic xanthoastrocytoma, sphenoid wing meningioma, subependymal giant cell astrocytoma, subependymoma, trilateral retinoblastoma, and nervous system metastasis of any origin.

114. The method of claim 106, wherein the infection is selected from adenoviral infections, hepatitis B, hepatitis G, poxvirus infections, herpesvirus infections, papillomavirus infections, HIV infections, meningitis (bacterial, fungal, or viral), bacterial persistence in brain, fungal persistence in brain, pancreatitis, and peritonitis.

115. A method for delivering an enzyme which has a deoxyribonuclease (DNase) activity to the nervous system in a subject in need thereof, comprising administering to the subject an expression vector comprising a nucleic acid comprising a promoter operably linked to a sequence encoding the enzyme, wherein the promoter is a nervous system-specific promoter.

116. A method for treating a disease associated with protein misfolding in a subject in need thereof, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter.

117. The method of claim 116, wherein the protein misfolding is associated with the presence of cell-free DNA (cfDNA) in cerebrospinal fluid (CSF) or nervous system tissue(s).

118. The method of claim 117, wherein the cfDNA is microbial and/or viral cfDNA.

119. The method of claim 118, wherein the microbial cfDNA is bacterial cfDNA.

120. The method of claim 118, wherein the viral cfDNA is bacteriophage cfDNA.

121. The method of any one of claims 115-120, wherein the disease associated with protein misfolding is selected from neurodegenerative diseases, neurodevelopmental diseases, psychiatric diseases, autoimmune diseases, oncological diseases and infections.

122. The method of claim 121, wherein the disease associated with protein misfolding is a neurodegenerative disease selected from Alzheimer's disease, Parkinson's disease, Amyotrophic Lateral Sclerosis (ALS), multiple sclerosis, CADASIL Syndrome, Friedreich's ataxia, cerebellar ataxia, spinocerebellar ataxia, dementia (e.g., fronto-temporal dementia, frontotemporal dementia with parkinsonism-17 (FTDP-17), familial Danish dementia, and familial British dementia), prion-caused diseases, Lewy body diseases, an amyloidosis (e.g., hereditary cerebral hemorrhage with amyloidosis, senile systemic amyloidosis, an amyloidosis with a hereditary cerebral hemorrhage, a primary systemic amyloidosis, a secondary systemic amyloidosis, a serum amyloidosis, a senile systemic amyloidosis, a hemodialysis-related amyloidosis, a Finnish hereditary systemic amyloidosis, an Atrial amyloidosis, a Lysozyme systemic amyloidosis, an Insulin-related amyloidosis, or a Fibrinogen α-chain amyloidosis), Spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm or dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia), Shy-Drager syndrome, spinal muscular atrophy, primary lateral sclerosis, hereditary spastic paraplegia, peroneal muscular atrophy (Charcot-Marie-Tooth), and hypertrophic interstitial polyneuropathy (Dejerine-Sottas). In one embodiment, the neurodegenerative disease is secondary to diabetes, rheumatoid arthritis, systemic lupus erythematosus (SLE), gout, metabolic syndrome, an amyloidosis, asthma, and prion disease.

123. The method of claim 121, wherein the disease associated with protein misfolding is a neurodevelopmental disease selected from autism, neural tube defects, attention deficit hyperactivity disorder, Dawn syndrome, cerebral palsy, Rett syndrome, Landau-Kleffner syndrome, Alexander disease, Alpers' disease, alternating hemiplegia, Angelman syndrome, ataxias and cerebellar or spinocerebellar degeneration, ataxia telangiectasia, attention deficit-hyperactivity disorder, autism spectrum disorders, Asperger syndrome, Batten disease, Canavan disease, Tourette syndrome, and impairments in vision and/or hearing.

124. The method of claim 121, wherein the disease associated with protein misfolding is a psychiatric disease selected from anxiety disorders, psychotic disorders, schizophrenia, bipolar disorder, depression, post-traumatic stress disorder, and epilepsy.

125. The method of claim 121, wherein the disease associated with protein misfolding is an autoimmune disease selected from autoimmune encephalitis, autoimmune-related epilepsy, central nervous system (CNS) vasculitis, Hashimoto's encephalopathy, steroid-responsive encephalopathy, neurosarcoidosis, Neuro-Behcet's disease, cerebral lupus, neuromyelitis optica, optic neuritis, diabetes, rheumatoid arthritis, systemic lupus erythematosus (SLE), gout, asthma, celiac disease, ankylosing spondylitis, vasculitis, pemphigus vulgaris, inflammatory bowel disease (IBD), Crohn's disease, and ulcerative colitis.

126. The method of claim 121, wherein the disease associated with protein misfolding is an oncological disease selected from astrocytoma, gliomas, glioblastoma, ependymoma, medulloblastoma, central nervous system (CNS) lymphoma, craniopharyngioma, glioblastoma, meningioma, pituitary carcinomas, pituitary adenomas, neurofibromatosis, embryonal tumors, tumors of the pineal region, tumors of meninges, choroid plexus tumors, neuronal and mixed neuronal-glial tumors, germ cell tumors, anaplastic astrocytoma, central neurocytoma, choroid plexus carcinoma, choroid plexus papilloma, dysembryoplastic neuroepithelial tumor, giant-cell glioblastoma, gliosarcoma, hemangiopericytoma, medulloepithelioma, neuroblastoma, neurocytoma, oligoastrocytoma, oligodendroglioma, optic nerve sheath meningioma, pilocytic astrocytoma, pinealoblastoma, pineocytoma, pleomorphic anaplastic neuroblastoma, pleomorphic xanthoastrocytoma, sphenoid wing meningioma, subependymal giant cell astrocytoma, subependymoma, trilateral retinoblastoma, and nervous system metastasis of any origin.

127. The method of claim 121, wherein the disease associated with protein misfolding is an infection selected from adenoviral infections, hepatitis B, hepatitis G, poxvirus infections, herpesvirus infections, papillomavirus infections, HIV infections, meningitis (bacterial, fungal, or viral), bacterial persistence in brain, fungal persistence in brain, pancreatitis, and peritonitis.

128. The method of any one of claims 115-120, wherein the disease associated with protein misfolding is selected from Alzheimer's disease, Parkinson's disease, Amyotrophic Lateral Sclerosis (ALS), multiple sclerosis, CADASIL Syndrome, stroke, Friedreich's ataxia, cerebellar ataxia, spinocerebellar ataxia, dementia, prion-caused diseases, Lewy body diseases, amyloidosis, spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski),corticodentatonigral degeneration, Hallervorden-Spatz disease, progressive familial myoclonic epilepsy, striatonigral degeneration, torsion dystonia, spasmodic torticollis and other dyskinesis, familial tremor, Gilles de la Tourette syndrome, cerebellar cortical degeneration, spinocerebellar degeneration, Shy-Drager syndrome, spinal muscular atrophy, primary lateral sclerosis, hereditary spastic paraplegia, peroneal muscular atrophy (Charcot-Marie-Tooth), hypertrophic interstitial polyneuropathy (Dejerine-Sottas), nervous system tumors, and secondary neurodegeneration.

129. The method of claim 128, wherein the nervous system tumors are selected from the group consisting of astrocytoma, gliomas, glioblastoma, ependymoma, medulloblastoma, CNS lymphoma, craniopharyngioma, glioblastoma, meningioma, pituitary carcinomas, pituitary adenoma, neurofibromatosis, embryonal tumors, tumors of the pineal region, tumors of meninges, choroid plexus tumors, neuronal and mixed neuronal-glial tumors, and germ cell tumors.

130. The method of claim 128, wherein the secondary neurodegeneration is selected from the group consisting of neurodegeneration resulting from destruction of neurons by neoplasm, edema, hemorrhage, stroke, trauma, immune attack, hypoxia, poisoning, metabolic defects, and infections.

131. A method for treating a neurodegenerative, neurodevelopmental, psychiatric, autoimmune or oncological disease or an infection in a subject in need thereof, said method comprising administering to the subject an expression vector comprising a nucleic acid comprising a promoter operably linked to a sequence encoding an enzyme which has a deoxyribonuclease (DNase) activity, wherein the promoter is a nervous system-specific promoter.

132. The method of claim 131, wherein the neurodegenerative disease is Alzheimer's disease, Parkinson's disease, or Amyotrophic Lateral Sclerosis (ALS).

133. The method of claim 131, wherein the neurodegenerative disease is secondary to diabetes, rheumatoid arthritis, systemic lupus erythematosus (SLE), gout, metabolic syndrome, an amyloidosis, asthma, or prion disease.

134. The method of claim 131, wherein the neurodegenerative disease is selected from Alzheimer's disease, Parkinson's disease, Amyotrophic Lateral Sclerosis (ALS), multiple sclerosis, CADASIL Syndrome, Friedreich's ataxia, cerebellar ataxia, spinocerebellar ataxia, dementia (e.g., fronto-temporal dementia, frontotemporal dementia with parkinsonism-17 (FTDP-17), familial Danish dementia, and familial British dementia), prion-caused diseases, Lewy body diseases, an amyloidosis (e.g., hereditary cerebral hemorrhage with amyloidosis, senile systemic amyloidosis, an amyloidosis with a hereditary cerebral hemorrhage, a primary systemic amyloidosis, a secondary systemic amyloidosis, a serum amyloidosis, a senile systemic amyloidosis, a hemodialysis-related amyloidosis, a Finnish hereditary systemic amyloidosis, an Atrial amyloidosis, a Lysozyme systemic amyloidosis, an Insulin-related amyloidosis, or a Fibrinogen a-chain amyloidosis), Spinal muscular atrophy, a bipolar disorder, schizophrenia, depressive disorder, autism, autism spectrum disorders, Chronic Fatigue Syndrome, Obsessive-Compulsive Disorder, generalized anxiety disorder (GAD), major depressive disorder (MDD), social anxiety disorder (SAD), attention-deficit/hyperactivity disorder (ADHD), Huntington's disease, Spinal muscular atrophy, Mild Cognitive Impairment (MCI), chronic traumatic encephalopathy (CTE), progressive supranuclear palsy (PSP), multiple system atrophy (MSA), corticobasal degeneration (CBD), agyrophilic grain disease (AGD), Pick's disease, olivopontocerebellar atrophy (OPCA), senile dementia of the Alzheimer type, progressive supranuclear palsy (Steel-Richardson-Olszewski), corticodentatonigral degeneration, Hallervorden-Spatz disease, progressive familial myoclonic epilepsy, striatonigral degeneration, torsion dystonia (e.g., torsion spasm or dystonia musculorum deformans), spasmodic torticollis and other dyskinesis, familial tremor, Gilles de la Tourette syndrome, cerebellar cortical degeneration, spinocerebellar degeneration (e.g., Friedreich's ataxia), Shy-Drager syndrome, spinal muscular atrophy, primary lateral sclerosis, hereditary spastic paraplegia, peroneal muscular atrophy (Charcot-Marie-Tooth), and hypertrophic interstitial polyneuropathy (Dejerine-Sottas). In one embodiment, the neurodegenerative disease is secondary to diabetes, rheumatoid arthritis, systemic lupus erythematosus (SLE), gout, metabolic syndrome, an amyloidosis, asthma, and prion disease.

135. The method of claim 131, wherein the neurodevelopmental disease is selected from autism, neural tube defects, attention deficit hyperactivity disorder, Dawn syndrome, cerebral palsy, Rett syndrome, Landau-Kleffner syndrome, Alexander disease, Alpers' disease, alternating hemiplegia, Angelman syndrome, ataxias and cerebellar or spinocerebellar degeneration, ataxia telangiectasia, attention deficit-hyperactivity disorder, autism spectrum disorders, Asperger syndrome, Batten disease, Canavan disease, Tourette syndrome, and impairments in vision and/or hearing.

136. The method of claim 131, wherein the psychiatric disease is selected from anxiety disorders, psychotic disorders, schizophrenia, bipolar disorder, depression, post-traumatic stress disorder, and epilepsy.

137. The method of claim 131, wherein the autoimmune disease is selected from autoimmune encephalitis, autoimmune-related epilepsy, central nervous system (CNS) vasculitis, Hashimoto's encephalopathy, steroid-responsive encephalopathy, neurosarcoidosis, Neuro-Behcet's disease, cerebral lupus, neuromyelitis optica, optic neuritis, diabetes, rheumatoid arthritis, systemic lupus erythematosus (SLE), gout, asthma, celiac disease, ankylosing spondylitis, vasculitis, pemphigus vulgaris, inflammatory bowel disease (IBD). Crohn's disease, and ulcerative colitis.

138. The method of claim 131, wherein the oncological disease is selected from astrocytoma, gliomas, glioblastoma, ependymoma, medulloblastoma, central nervous system (CNS) lymphoma, craniopharyngioma, glioblastoma, meningioma, pituitary carcinomas, pituitary adenomas, neurofibromatosis, embryonal tumors, tumors of the pineal region, tumors of meninges, choroid plexus tumors, neuronal and mixed neuronal-glial tumors, germ cell tumors, anaplastic astrocytoma, central neurocytoma, choroid plexus carcinoma, choroid plexus papilloma, dysembryoplastic neuroepithelial tumor, giant-cell glioblastoma, gliosarcoma, hemangiopericytoma, medulloepithelioma, neuroblastoma, neurocytoma, oligoastrocytoma, oligodendroglioma, optic nerve sheath meningioma, pilocytic astrocytoma, pinealoblastoma, pineocytoma, pleomorphic anaplastic neuroblastoma, pleomorphic xanthoastrocytoma, sphenoid wing meningioma, subependymal giant cell astrocytoma, subependymoma, trilateral retinoblastoma, and nervous system metastasis of any origin.

139. The method of claim 131, wherein the infection is selected from adenoviral infections, hepatitis B, hepatitis G, poxvirus infections, herpesvirus infections, papillomavirus infections, HIV infections, meningitis (bacterial, fungal, or viral), bacterial persistence in brain, fungal persistence in brain, pancreatitis, and peritonitis.

140. The method of any one of claims 108-119, wherein the vector further comprises one or more molecules capable of targeting of the nucleic acid to nervous system.

141. The method of any one of claims 115-140, wherein the nervous system-specific promoter mediates a substantially increased expression of the enzyme in the nervous system as compared to other tissues and organs.

142. The method of any one of claims 90-141, wherein the subject is human.


SEQUENCE LISTING (51 sequences)

SEQ ID NO: 1 - protein, 282 aa, Homo sapiens [sequence omitted]

SEQ ID NO: 2 - protein, 282 aa, Artificial Sequence
  Description of Artificial Sequence Synthetic polypeptide
MRGMKLLGALLALAALLQGAVSLKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 3 - protein, 732 aa, Artificial Sequence
  Description of Artificial Sequence Synthetic polypeptide
  MOD_RES (166)..(166): K or R
  MOD_RES (202)..(202): A or S
  MOD_RES (263)..(263): A or G
  MOD_RES (308)..(308): R or K
  MOD_RES (408)..(408): E or Q
  MOD_RES (456)..(456): T or E
  MOD_RES (489)..(489): A or T
  MOD_RES (558)..(558): S or N
  MOD_RES (572)..(572): Q or E
  MOD_RES (583)..(583): S or A
  MOD_RES (605)..(605): N or D
AADGYLPDWLEDNLSEGIREWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAXKRLNFGQTGDSESVPDPQPLGEPPAAPSGVGSNTMXAGGGAPMADNNEGADGVGNASGNWHCDSTWLGDRVITTSTRTALPTYNNHLYKQISSQSGXSTNDNTYFGYSTPWGYFDFNRFHCHFSPRDWQRLINNNWGFRPKXLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFXFSYTFEDVPFHSSYAHSQSLDRLNPLIDQYLYYLSRTQTTSGTAGNRXLQFSQAGPSSMANQAKNWLPGPCYRQQRVSKTXNQNNNSNFAWTGATKYHLNGRDSLVNPGPAMATHKDDEDKFFPMSGVLIFGKQGAGNSNVDLDNVMITXEEEIKTTNPVATEXYGTVATNLQSXNTAPATGTVNSQGALPGMVWQXRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 4 - protein, 260 aa, Homo sapiens
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 5 - protein, 260 aa, Artificial Sequence
  Description of Artificial Sequence Synthetic polypeptide
LKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 6 - protein, 22 aa, Homo sapiens
MRGMKLLGALLALAALLQGAVS

SEQ ID NO: 7 - protein, 20 aa, Unknown
  Description of Unknown IL2 secretory signal sequence
MYRMQLLSCIALSLALVTNS

SEQ ID NO: 8 - DNA, 214 bp, Homo sapiens
actagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacc

SEQ ID NO: 9 - protein, 726 aa, Artificial Sequence
  Description of Artificial Sequence Synthetic polypeptide
  MOD_RES (165)..(165): K or R
  MOD_RES (201)..(201): A or S
  MOD_RES (261)..(261): A or G
  MOD_RES (306)..(306): R or K
  MOD_RES (406)..(406): E or Q
  MOD_RES (454)..(454): T or E
  MOD_RES (486)..(486): A or T
  MOD_RES (554)..(554): S or N
  MOD_RES (568)..(568): Q or E
  MOD_RES (579)..(579): S or A
  MOD_RES (600)..(600): N or D
AADGYLPDWLEDNLSEGIREWDLKPGAPKPKANQQKQDDGRGLVLPGYYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAXKRLNFGQTGDSESVPDPQPLGEPPAAPSGVGSNTMXAGGGAPADNNEGADGVGNASGNWHCDSTWLGDRVITTSTRTALPTYNNHLYKQISSQSGXSTNDNTYFGYSTPWGYFDFNRFHCHFSPRDWQRLINNNWGFRPKXLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFXFSYTFEDVPFHSSYAHSQSLDRLNPLIDQYLYYLSRTQTTSGTAGNRXLQFSQAGPSSANQAKNWLPGPCYRQQRVSKTXNQNNNSNFAWTGATKYHLNGRDSLVNPGPAMATHKDDEDKFFPMSGVLIFGKQGAGNSNVDLDNVITXEEEIKTTNPVATEXYGTVATNLQSXNTAPATGTVNSQGALPGVWQXRDVYLQGPIWAKIPHTDGHFHPSPLMGGFGLKHPPPQILIKNTPVPANPPTTFSPAKFASFITQYSTGQVSVEIEELQKENSKRWNPEIQYTSNYNKSTNVDFAVDTNGVYSEPRPIGTRYLTRNL

SEQ ID NO: 10 - DNA, 20 bp, Artificial Sequence
  Description of Artificial Sequence Synthetic primer
caacttcatccacgttcacc

SEQ ID NO: 11 - DNA, 21 bp, Artificial Sequence
  Description of Artificial Sequence Synthetic primer
gttctgagctccaaccattct

SEQ ID NO: 12 - DNA, 21 bp, Artificial Sequence
  Description of Artificial Sequence Synthetic primer
cactgtgggtccttcatcttt

SEQ ID NO: 13 - DNA, 50 bp, Artificial Sequence
  Description of Artificial Sequence Synthetic primer
  modified_base (42)..(42): a, c, t, g, unknown or other
tcgtcggcagcgtcagatgtgtataagagacagcctacgggnggcwgcag

SEQ ID NO: 14 - DNA, 55 bp, Artificial Sequence
  Description of Artificial Sequence Synthetic primer
gtctcgtgggctcggagatgtgtataagagacaggactachvgggtatctaatcc

SEQ ID NO: 15 - DNA, 397 bp, Homo sapiens
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 16 - DNA, 637 bp, Artificial Sequence
  Description of Artificial Sequence Synthetic polynucleotide
agtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 17 - DNA, 320 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagg

SEQ ID NO: 18 - DNA, 840 bp, Artificial Sequence
  Description of Artificial Sequence Synthetic polynucleotide
atgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 19 - DNA, 849 bp, Artificial Sequence
  Description of Artificial Sequence Synthetic polynucleotide
atgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 20 - DNA, 66 bp, Homo sapiens
atgaggggcatgaagctgctgggggcgctgctggcactggcggccctactgcagggggccgtgtcc

SEQ ID NO: 21 - DNA, 783 bp, Artificial Sequence
  Description of Artificial Sequence Synthetic polynucleotide
ctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 22 - DNA, 846 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaag

SEQ ID NO: 23 - DNA, 780 bp, Homo sapiens
ctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaag

SEQ ID NO: 24 - protein, 284 aa, Mus musculus
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 25 - protein, 22 aa, Mus musculus
MRYTGLMGTLLTLVNLLQLAGT

SEQ ID NO: 26 - protein, 262 aa, Mus musculus
LRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 27 - DNA, 66 bp, Mus musculus
atgcggtacacagggctaatgggaacactgctcaccttggtcaacctgctgcagctggctgggact

SEQ ID NO: 28 - DNA, 789 bp, Mus musculus
ctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctga

SEQ ID NO: 29 - DNA, 855 bp, Mus musculus
atgcggtacacagggctaatgggaacactgctcaccttggtcaacctgctgcagctggctgggactctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctga

SEQ ID NO: 30 - DNA, 2212 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 31 - DNA, 2212 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 32 - DNA, 849 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 33 - DNA, 9 bp, Artificial Sequence
  Description of Artificial Sequence Synthetic oligonucleotide
gccgccacc

SEQ ID NO: 34 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 35 - protein, 730 aa, Artificial Sequence
  Description of Artificial Sequence Synthetic polypeptide
MAADGYLPDWLEDNLSEGIREWWDLKPGAPKPKANQQKQDDGRGLVLPGYYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSANQAKNWLPGPCYRQQRVSKTTNQNNNSNFAWTGATKYHLNGRDSLVNPGPAMATHKDDEDKFFPMSGVLIFGKQGAGNSNVDLDNVITNEEEIKTTNPVATEEYGTVATNLQSANTAPATGTVNSQGALPGVWQDRDVYLQGPIWAKIPHTDGHFHPSPLMGGFGLKHPPPQILIKNTPVPANPPTTFSPAKFASFITQYSTGQVSVEIEELQKENSKRWNPEIQYTSNYNKSTNVDFAVDTNGVYSEPRPIGTRYLTRNL

SEQ ID NO: 36 - DNA, 448 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 37 - DNA, 768 bp, Artificial Sequence
  Description of Artificial Sequence Synthetic polynucleotide
gcggccgctctagagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggt

SEQ ID NO: 38 - DNA, 664 bp, Artificial Sequence
  Description of Artificial Sequence Synthetic polynucleotide
cctctgtctgtctgtctgtctctttggtgtgatgtatgtggtgtgtgtgtattctacaaggttgacatgatgacagaatttaattttcttagcagcaagctcatggatcctggtgataaatgcagcatgactttactgaaaaggctttgtgatcttgaagagtggattgacttcactgtcggcagcacatgcaatctcacttgtttggtgtaatgaaagaagagaatgagaggtggaagggggatggtaatgttgaaaaaaagaatggtacagaggaaactgaggttggagagagatggggtagatggtaagagatggagaaagagggaaggaaatggagagaaagacagagagacagagagagacacacagagagacacacagagacagagaggaagggaaagggaaagagaaaggaagaggaagagggggaggggaaggggaaggggaaggggaagggagagggagaaatgtggacactagccagatttaagggagaaattagggggttgccagtctgtccacctctgatggtggcaactcagcagaaagctgctgggctcagtctggctttgttgagcaaccctgactccaccccttttcttccccacaaagcaagcttttaaagggaaggctttcttcattgaatgactgccacagtacg

SEQ ID NO: 39 - DNA, 1497 bp, Artificial Sequence
  Description of Artificial Sequence Synthetic polynucleotide
gggggtgagcgagggctgctgggaccattgcagggaacaatgataatctaggcttggttcctacccagagagcacgcactcatccttcatgcactcccctgttccaaaccctcactggctccgtactgcctccgaccttccgagactttagcctggctcctgtcaacatctctgacccttactacatgatcctctctttggtccatgctccagcctaatctaattgcggtggcttgtgcgtggtggcattcccagccaccatacctttacccacgctggtccttccatgcggaatgcctttccagggcctgctttgcccgcttctgctcatacacaggcatgccctccaggatggcttcctacctctttcccttgggggattgatctctctgtcttggggttctcggagcccttgacctgacccctttctgtttggcaaaaaagtaatttacctcggtgtccttctccctggtagtctgtgagctccccaaggctgggctgtgcctgattcacctctggaacttgcttagcacagtgcgtggcctgctgcaggtgttcattgagcacttgccgaatgaatgcatgaatgaatgaatgaatgaatgaatgcaaggggctgctaatccacaggactcctcaggtcagccagacgtcccggttccaaggcctgccactgactcacctcaggaccctgcttgaaccattagaactcaccctgcctcactttccccctctgtgaaatggggctccaactcctattcaagctactatcatttgggggcattgtgaggccacagatcccagaacatcagagtcagaggtagcccagaaagcttcccacccatccctacaaatgggaaactgaggtctggagagggaagggcagagttgggctccctgtctcaggctcggacccaccatcaggcctgtctctaaaacgaatcccagctcccacgctgcaccctgagcctggaagcctgagccacacaaggacggggaattttccttcccacttccagaggcctctgaacctccctgagcttgtcccctttggagggtattgggcagcagcgtgggcagaaccccagctcactgtctgggggagcgctgcaggacagccttgtctgtctgtctcagcctgccctggggacccgaggtcagggaggaagtgccgcatctggtcttccccagagcgagagtgtgagcaagggtgggattgcgtgtggcccgagagtagcccctcccctccccctgtccccaccccaaaccctcttaatgaaatcaagctggccctgcggcccagccggggagggaggaaggaggagggacgggaggagggacgggaggagggagggcgggcaggcgccagcccagagcagccccgggcaccagcacggactctctcttccagcccaggtgccccccactctcgctccattcggcgggagcacccagtcctgtacgccaaggaactggtgagtcctggggtcccctcct

SEQ ID NO: 40 - DNA, 1515 bp, Artificial Sequence
  Description of Artificial Sequence Synthetic polynucleotide
acatgcaggcactgtggccccgaaaaactcttcagaggtgagcggctttcaggatgagagggccaacaatggctcatctcctaaagacctcgataaacataatcagtagccccagaaaacacaacagctgtcccaagcctatcccctttgcacctatctcagaaaaggcaacatgcagagtggtcaggagttcaagtcctcagggccctactctgaagctgtgaccctgacaggtcactttagcgctcagcttccctttcctcatctgtaaaatgggaatagaaactgaatctgcttcatggggccttgtaagtattacataagctgtcatatgcaaaatgtccatcacatagttcaccgccccaggcatcctagaaactattaagtcacgataaggaggttgctactgtcctgggacctggggagctcggctgtccagtccgaaagggccttctcgccctcgagaccccttcaagtccctttctctccgggcccgggtcccagagtccccttgtgcttgctcctgagggtgacctgcccgtgccccggggcgagcacaggtgcggccctggccacaatgcgggcgtggcgagccgacacccggggaggccaagggcgcctcgagggagggagggaggaggctgacccgggcgtcccgaaggacccgcccggccccggactccgggcggcagccggcctcgcgccccgcccccgccccgggagccccgcccagtcccgtcgcgccgcgccgccccaccgcccgggttggctgccctggaggccacgcgcggcgatttgccacgcgccgcgtcaccgggccgctcccggcctgggctcccgggggctggtcagggaggtggcggcggctgagcggcggagcgggggcggcagggcgcgacgccgccgggccgccccgccctgggaggcgcccgggccctcatcaagtgaccagtatcccttccaggggaacacggtccttcagaggaaagcgagctccaacccgcggccccggcgccaagccgccgtcatcttcttcctgtgccaggtgatcgtctcctcacccacccggaaaaacatagtccgcccccacgtccttgtggaatagcgcccgcttccaagcaccgtgactctctttgccctacccttgtctttcccattccaattactctagaacccaccgagaggataattcagtcctgaaaagaaactgatggggagaagcgaggaagggaacccaggagggaggggaggcagggctgcggagggacaccgaggcggcggaggtagtgcgcacgcgcagcacagaacgagttccggtctggccgaggcttgtctcctaaaaatagccccggtgtggggatccgtgcgcggatgtcccggcgagtcccgggctgaaagaggcggctccgggcggcgcgaagcgctggtggcgggcccgggctgcggcgtgtgcgcgcccgccagctgctccggagatacggtgagggccgcgggcagcggggctcagtccgcgac

SEQ ID NO: 41 - DNA, 1545 bp, Artificial Sequence
  Description of Artificial Sequence Synthetic polynucleotide
agtgcagcctcaggggttgaaggtgaaatgtgaaatttgctgtaattacttacccaaaatgtctgtattaaaggctattagaagcagttattttaagtacttctttatacatctttccctcttctgtttagtatttaaatgtgcaacaaaagtagtggcagatgttactcatctgaagaaactaaaacaagataagtcagtggcaggagtggtggccggttaaagatctcttcgaagtgctttgttcagtaactgttgttttgtctcacattaagttgtaggtggggtgctgttaaagcttgtccactcacgatggtgttcagctgagacctgtgagttcaagttgtcttgcccagagttaaaggccataaaactaagacagtgtcgttcctgtcttgggtgtaactcctaaaaaactagtcagttcttgcaataggaggaaaattattgcaaataaacctttgatttaccagcttcagtaagttgttcattctgtctctgtagctgtagtttttaccttaacagtttgaggctaccatttctcttcctttcatgcttatggggtccacacaaaccctgctaggctacctacagtataaaatcttaaaactccatgtggtgttgtgagctggtagcagatggactttcttttgcatctgctgtgtacagacttggctttgtactcttttcctgggggagagaaagtctttcttctctgctagttgattcctgtttttgtgaaacagctaagaaaacatctgtggcaggcagaaaaattgacaaaggattgacaaacaccattagctgaaaattttccatttcattgcaactatgcattgtttactttactgtaaatatcttaatacatcatcctctgaatatgctggcagagaagctggagaactgtgatttcaattaaggttagtaattgatggtatctagtgttcaaaggctgaagcttgattaacatctgcttggacaaattgtcattgtgaagtggttttgatgtgcattgaagattatttctttctggcttatctgatgttgtggctgtgaaatgcttgtcttgggtttgcttatttttgtaaactacttcctttggctgtaaattgcagagcactggagctttaccaaaagtgcagtgtataatggtaagcttgtcctaataagggaagcaagaagtgtatttatcacagacatgaaagctaaccgaggacttgagagactcaaactggtgcttttgtctctctctctctgtctttctctctctcacacacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgt

SEQ ID NO: 42 - DNA, 2030 bp, Artificial Sequence
  Description of Artificial Sequence Synthetic polynucleotide
atgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 43 - DNA, 2030 bp, Artificial Sequence
  Description of Artificial Sequence Synthetic polynucleotide
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 44 - DNA, 849 bp, Artificial Sequence; identical to SEQ ID NO: 19
  Description of Artificial Sequence Synthetic polynucleotide

SEQ ID NO: 45 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 5 aa, Adeno-associated virus
NGTSG

SEQ ID NO: 48 - protein, 4 aa, Artificial Sequence
  Description of Artificial Sequence Synthetic peptide
SGTH

SEQ ID NO: 49 - protein, 4 aa, Artificial Sequence
  Description of Artificial Sequence Synthetic peptide
SDTH

SEQ ID NO: 50 - protein, 5 aa, Adeno-associated virus
GGTAN

SEQ ID NO: 51 - protein, 5 aa, Artificial Sequence
  Description of Artificial Sequence Synthetic peptide
DGSGL